(19) United States
(12) Patent Application Publication
(10) Pub. No.: US 2009/0054376 A1
(43) Pub. Date: Feb. 26, 2009

(54) BENZOFURAN DERIVATIVES WITH THERAPEUTIC ACTIVITIES

(75) Inventors: Avihai Yacovan, Gedera (IL); Flavio Grynszpan, Mazkeret Batya (IL); Alexander Aizikovich, Ramat Gan (IL); Marcus Stephen Brody, Los Angeles, CA (US); Avi Bar-Joseph, Rehovot (IL); Sigal Meilin, Givatayim (IL)

Correspondence Address: WINSTON & STRAWN LLP;PATENT DEPARTMENT, 1700 K STREET, N.W., WASHINGTON, DC, 20006, US

(73) Assignee: Pharmos Corporation

(21) Appl. No.: 11/915,825

(22) PCT Filed: May 31, 2006
(86) PCT No.: PCT/IL06/00641

§ 371 Date: May 20, 2008

Related U.S. Application Data
(60) Provisional application No. 60/685,374, filed on May 31, 2005.

(51) Int. Cl. A61K 31/665; C07D 307/91; A61K 31/343; C07F 9/655; A61P 35/00; A61K 31/5377; C07D 413/12

(52) U.S. Cl. 514/100; 549/460; 514/468; 514/232.8; 544/153; 549/220

(57) ABSTRACT

The present invention relates to novel benzofuran compounds, to pharmaceutical compositions that include such compounds, and to methods of use thereof. Certain compounds of the invention share some pharmacological properties with cannabinoids and have a common wide range of beneficial therapeutic indications. In particular, compounds of the invention are useful as analgesic, neuroprotective, immunomodulatory and anti-inflammatory agents.

### FIELD OF THE INVENTION

The present invention relates to novel benzofuran derivatives, to pharmaceutical compositions comprising same, and to methods of use thereof. In particular, compounds of the invention are useful as analgesic, neuroprotective, immunomodulatory and anti-inflammatory agents.

### BACKGROUND OF THE INVENTION

Cannabis was historically used for the treatment of insomnia, inflammation, pain, various psychoses, digestive disorders, depression, migraine, neuralgia, fatigue, constipation, diarrhea, parasites, infections and appetite disorders. Some of the potential medical uses of cannabis have generated voluminous scientific literature reviewed by Pate [Pate D. W., Journal of the International Hemp Association 2(2): 74-6, 1995]. Originally defined as any individual bioactive component of the plant cannabis, the cannabinoids have come to encompass their endogenous counterparts and any synthetic compound that would exert most of its actions via the activation of the specific G-protein coupled cannabinoid receptors. To date, two cannabinoid receptors have been cloned and characterized, cannabinoid receptor type 1 (CB₁) and cannabinoid receptor type 2 (CB₂), although additional receptors may exist [Begg M. et al., Pharmacology & Therapeutics 106: 133-145, 2005]. The CB₁ receptors are predominantly found in the central nervous system (CNS) and are responsible for the psychotropic effects of cannabinoids, whereas the CB₂ receptors are expressed mainly in the periphery on immune cells.

Owing to their wide range of therapeutic activity, cannabinoids have often been considered for the development of new medications. Moreover, the isolation and synthesis of the major psychoactive constituent of cannabis, Δ⁹-tetrahydrocannabinol (Δ⁹-THC), has open the way to medicinal chemists for the preparation of numerous synthetic cannabinoids. The identification of the cannabinoid receptors and the elucidation of their respective roles and distribution have prompted the rational design of compounds which could dissociate between the therapeutic potential and the adverse effects.

Still, the cannabinoids developed to date, including for example Δ⁹-THC prescribed today as an anti-emetic agent, suffer from certain drawbacks that might include any of the following: the psychoactive side effects and the legal concerns they arise, the complexity of synthesis and the resulting cost of production, the lack of water-solubility and the ensuing formulatory problems, and the lack of oral bioavailability and its implication regarding the possible routes of administration and patient compliance.

It would therefore be advantageous to develop new types of cannabinoids which would lack cannabimimetic side-effects, or at least provide a higher therapeutic index, and would be easier to prepare both as a drug substance and as a drug product.

Following the discovery of the cannabinoid receptors, it was assumed that all cannabinoid-induced activities could from thereon be fully explained by receptor-mediated mechanisms. However, it was acknowledged that some of the beneficial activities of the cannabinoids are not mediated by the two identified CB₁ and CB₂ receptors. This observation has led to the inclusion in the class of cannabinoids of compounds which do not bind to either known cannabinoid receptors and are farther related to the more classical cannabinoids. For example certain metabolites, reagents or by-products derived or used in the preparation of traditional cannabinoids are often themselves referred as cannabinoids.

Cannabifuran (CBF) and Cannabielsoin (CBE), both naturally occurring benzofuran derivatives depicted in Scheme 1, are examples of such non-classical cannabinoids, for which there is little or no pharmacological information.

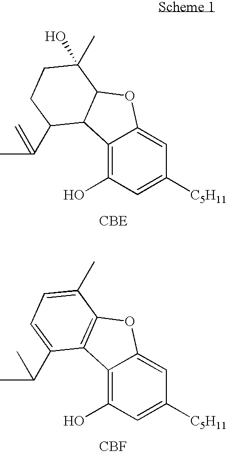

Cannabifuran is a minor constituent of cannabis sativa and a naturally occurring dibenzofuran which lacks the classical structure of tetrahydrocannabinol (THC). Due to its minute availability, little is known about its biological activity. The few naturally occurring dibenzofuran compounds identified to date in other plants were reported to have phytoalexin, antifingal and antibiotic properties. Some articles have addressed the issue of the synthesis of cannabifuran, for example Sargent et al. and Serra et al. [Sargent M. V. et al., J. Chem. Soc. Perkin Trans. I 7: 1605-10, 1982; Serra S. et al. Synlett 13: 2005-8, 2003], however no biological activity was reported.

Cannabielsoin is a component of marijuana which was also identified as a minor metabolite of cannabidiol (CBD) in liver microsomes and in vivo. The information available concerning its activity suggests that CBE does not primarily act, if at all, through CB₁ mediated mechanisms, since at 10 mg/kg i.v. it does not affect body temperature of mice, nor does it prolong pentobarbital-induced sleep [Yamamoto I. et al., Pharmacology, Biochemistry & Behavior 40: 541-546, 1991]. Certain derivatives of cannabielsoin were prepared for analytical purposes.

The existence of CBF and CBE indicates that compounds harboring a benzofuran structure might be considered as cannabinoids and that this scaffold could be used for the preparation of novel compounds which might have the therapeutic advantages common to other cannabinoids.

EP 1206934 discloses that certain phenol derivatives that encompass specific dibenzofuran compounds, including cannabifuran, may be used for the blockade of sodium channels and/or for influencing the kinetics of sodium channels. The inventors also suggest cosmetic use for peeling of the epidermis. However, the experiments were carried out with substituted phenols lacking a fused furan ring, such as 3-methylphenol, 4-chlorophenol and selected derivatives.

U.S. Pat. No. 4,960,815 discloses that certain benzofuran amines can be used as chemical intermediates for the preparation of isotopically-labeled derivatives useful for diagnosing neurodegenerative disorders. Most of the preferred intermediates disclosed therein have a non-substituted phenyl moiety, and there is no teaching concerning the activity of these intermediates.

International patent applications Nos. WO 00/08007, WO 00/07579 and WO 03/045375, all assigned to Bayer, disclose the preparation of cyclopentabenzofuran derivatives and use thereof for the treatment of nuclear factor κB-dependent diseases. These compounds have fixed substituents, namely hydroxyl and phenyl optionally substituted, on the carbon atoms linking the furan ring to the fused cyclopentan ring. The hydroxyl and aryl, which are positioned at C1 and C2 according to the nomenclature adopted in the present application, are in cis configuration to one another. Moreover, these cyclopentabenzofuran derivatives harbor an additional phenyl at the adjacent position C3.

DE 199 34 952 assigned to Novartis also refers to cyclopentabenzofuran derivatives. Though very broadly claimed, the specification discloses only compounds wherein the phenyl ring of the benzofuran moiety is preferably substituted by methoxy groups. As in the case of the Bayer applications, the compounds of DE 199 34 952 have a fixed phenyl group at position C2. Moreover, these specific compounds are attributed agro-chemical use as acaricides and insecticides, and are not contemplated as medicaments.

Cardillo et al. [Cardillo B. et al., Gazetta Chimica Italiana 103: 127-39, 1973] discloses a synthetic method for the preparation of cannabinoids, including benzofuran derivatives, based on the alkylation of resorcinols with monoterpenoid alcohols. However, the use of menth-3-en-5-ol or pulegol to alkilate orcinol yields isomers wherein the substituents of the fused cyclohexan ring are limited to isopropyl at position C2 and methyl at position C5, according to the present nomenclature. No biological activity is disclosed for any of the four isomers prepared.

Cannabinoids are useful candidates for the treatment of numerous therapeutic indications, but most still suffer from certain shortcomings. Despite the progress achieved with such compounds, it would be advantageous to prepare new compounds which would ally to a therapeutic benefit for a wide range of disease states, ease of preparation and improved safety.

### SUMMARY OF THE INVENTION

The present invention provides new benzofuran derivatives, pharmaceutical compositions comprising same and methods of use thereof.

Compounds of the present invention may be considered as non-conventional cannabinoids and, like more traditional cannabinoids, the new benzofuran derivatives of the invention can act through agonistic or antagonistic modulation of cannabinoid receptors and/or through non-cannabinoid receptor or non-receptor mediated mechanisms. The therapeutic effects may inter alia include anti-inflammatory, immunomodulatory, neuroprotective, analgesic, anti-neoplastic, cardioprotective and anti-osteoporosis activities.

The compounds of the invention can possess one or more chiral centers, and can therefore be produced as individual stereoisomers such as enantiomers and diastereomers or as mixtures, racemic or otherwise, of stereoisomers, depending on synthetic conditions and appropriate separation and isolation. All of these individual stereoisomers and mixture thereof are intended to be included within the scope of the present invention.

According to a first aspect, the present invention provides a compound of formula (I):

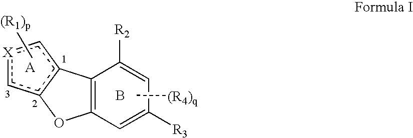

wherein [symbol]
represents a single or double bond;
X is (CHₘ)ₙ wherein in is an integer from, 0 to 2 and n is an integer from 0 to 4;
R₁ is at each occurrence selected independently from the group consisting of:
a) a halogen;
b) a carbonyl;
c) an aryl;
d) Rₐ wherein Rₐ is selected from the group consisting of R_{b}, OR_{b}, C(O)OR_{b} and OC(O)R_{b} wherein R_{b} is a saturated or unsaturated, linear or branched C₁-C₈ alkyl substituted with one or more heteroatoms selected from the group consisting of N, O and S;
e) R_{c}, wherein R_{c} is selected from R, OR, OC(O)OR, C(O)OR, OC(O)R and OC(O)N(R′)₂, wherein R is selected from the group consisting of a hydrogen, a saturated or unsaturated, linear, branched or cyclic C₁-C₆ alkyl, C₁-C₆ alkyl-OR′, C₁-C₆ alkyl-(OR′)₂, C₁-C₆ alkyl-C(O)OR′, and C₁-C₆ alkyl-C(O)N(R′)₂, and wherein R′ is at each occurrence independently selected from the group consisting of a hydrogen and a saturated or unsaturated, linear, branched or cyclic C₁-C₆ alkyl;
f) an oxime; and
g) N(R′)₂, wherein R′ is at each occurrence as previously defined;
p is an integer from 0 to 14;
   R₂ is selected from the group consisting of:
a) a hydrogen;
b) Rₐ or R_{c}, wherein Rₐ and R_{c} are as previously defined; and
c) OR″Z, wherein R″ is selected from the group consisting of a direct bond, C(O), Rₑ and C(O)Rₑ wherein Rₑ is a saturated or unsaturated, linear or branched C₁-C₈ alkyl, and Z is selected from the group consisting of ONO₂, a halogen, P(O)(OR′)₂, SR′, S(O)R′, S(O)(O)R′, N(R′)₂, wherein R′ is as previously defined, and a saturated or unsaturated heterocyclic ring of up to 6 atoms containing at least one heteroatom selected from the group consisting of N, O and S;
   R₃ is selected from the group consisting of:
a) R_{d} wherein R_{d} is selected from the group consisting of hydrogen, C(O)OR′″, C(O)R′″, CN and NO₂, wherein R′″ is selected from the group consisting of a hydrogen and a saturated or unsaturated, linear, branched or cyclic C₁-C₁₂ alkyl;
b) a saturated or unsaturated, linear, branched or cyclic C₂-C₁₂ alkyl which is unsubstituted or substituted by a saturated or unsaturated heterocyclic ring as previously defined;
c) a saturated or unsaturated, linear or branched C₁-C₁₂ alkyl substituted by an aryl; and
d) a saturated or unsaturated heterocyclic ring as previously defined, said ring being unsubstituted or substituted by at least one saturated or unsaturated, linear branched or cyclic C₁-C₆ alkyl, wherein said alkyl can be unsubstituted or substituted by an aryl; and
   R₄ is selected independently at each occurrence from the group consisting of hydrogen, NO₂ and NH₂; and q is an integer from 0 to 2; and stereoisomers, pharmaceutically acceptable salts, esters, polymorphs or solvates of said compounds;
   with the provisos that (a) A is not a phenyl ring; (b) when n is 1, R₁ is not a phenyl at position C2; (c) when n is 2, and R₁ at C2 is isopropyl then R₁ at C5 is other than methyl; and (d) when n is 2, R₁ is methyl and hydroxyl at C3 and isopropenyl at C6, then R₂ is other than OH, OCH₃ and OC(O)CH₃.

According to certain embodiments, the present invention provides a compound of formula (I) as defined therein, wherein n is an integer from 1 to 3, p is an integer from 0 to 4, q is an integer from 0 to 2, ring A is saturated or unsaturated wherein the optional double bond on ring A is positioned between C1 and C2 or C3 and C4, R₁ is at each occurrence independently selected from the group consisting of hydrogen, halogen, carbonyl, oxime, NH₂, R, C(O)OR, and OR; R₂ is selected from the group consisting of hydrogen, R_{c}, OR, OR″Z, OC(O)R_{b}, OR_{b} and OC(O)R; R₃ is selected from the group consisting of a saturated or unsaturated, linear, branched or cyclic C₁-C₁₂ alkyl which is unsubstituted or substituted by a heterocyclic ring or by an aryl, C(O)R′″ and C(O)OR′″; and R₄ is selected from the group consisting of hydrogen and NO₂, wherein R, R″, R′″, R_{b}, heterocyclic ring and Z are as previously defined.

According to additional embodiments, the present invention provides a compound of formula (I) as defined therein, wherein:

n is 1, ring A is saturated, R₁ is at each occurrence independently selected from the group consisting of hydrogen and CH₃, R₂ is OH or OC(O)CH═CHC(O)OH, and R₃ is selected from the group consisting of 1,1-dimethylpentyl and 1,1-dimethylheptyl;
n is 2, ring A is saturated or unsaturated wherein the optional double bond is positioned between C1 and C2 or C3 and C4, R₁ is at each occurrence independently selected from the group consisting of hydrogen, carbonyl, isopropylidene, oxime, iodine, OH and CH₃, R₂ is selected from the group consisting of OH, OCH₃, OCH₂C(O)OH, OCH₂SCH₃, OP(O)(OH)₂, OP(O)(OC₂H₅)₂, OCH₂-tetrazole, OCH₂CH₂-morpholine, OCH₂CH(OH)CH₂OH, OC(O)CH═CHC(O)OH, OC(O)CH₃, OC(O)(CH₂)₂NHCH₃, OC(O)-piperidine, OC(O)(CH₂)₃Br and OC(O)(CH₂)₃ONO₂, R₃ is selected from the group consisting of 2-phenethyl-[1,3]-dithiolane, 2-methyl-[1,3]dithiolan-2-yl, C(O)CH₃, C(O)OCH₃, 1,1-dimethylpentyl and 1,1-dimethylheptyl and R₄ is selected from the group consisting of hydrogen and NO₂;
n is 3, ring A is saturated or unsaturated wherein the optional double bond is between C3 and C4, R₁ is selected from the group consisting of hydrogen, iodine, NH₂, OH, OC(O)CH═CHC(O)OH, C(O)OCH₃, C(O)OH, CH₂OH, CH₂C(O)OCH₃, oxime, and carbonyl, R₂ is selected from the group consisting of hydrogen, OH, OCH₂CH₂-morpholine, OP(O)(OH)₂, OCH₂C(O)OH, OC(O)CH═CHC(O)OH, OCH₂-tetrazole, OC(O)CH₂OCH₂CH₂OCH₂CH₂OCH₃, OCH₂C(O)N(C₂H₅)₂ and O(CH₂)₃C(O)OH, and R₃ is selected from the group consisting of pentyl, 1,1-dimethylpentyl and 1,1-dimethylheptyl.

According to exemplary embodiments, the present invention provides a compound of formula (I) wherein:

n is 1, ring A is saturated, R₁ is hydrogen, CH₃ at position C2, or CH₃ at positions C2 and C3, R₂ is OH and R₃ is 1,1-dimethylheptyl;
n is 1, ring A is saturated, R₁ is CH₃ at position C2, R₂ is OH and R₃ is 1,1-dimethylpentyl;
n is 1, ring A is saturated, R₁ is CH₃ at position C2 and C3, R₂ is OC(O)CH═CHC(O)OH and R₃ is 1,1-dimethylheptyl;
n is 2, ring A is saturated, R₁ is selected from the group consisting of hydrogen, OH, carbonyl, iodine or oxime at position C3, gem-dimethyl at position C4, CH₃ at position C2 and isopropylidene at position C5, carbonyl at position C3 and gem-dimethyl at position C4 and both OH at position C3 and gem-dimethyl at position C4, R₂ is OH, and R₃ is 1,1-dimethylheptyl;
n is 2, ring A is saturated, R₁ is selected from the group consisting of hydrogen, OH, carbonyl or oxime at position C3 with or without a further gem-dimethyl at position C4, iodine at position C3 and gem-dimethyl at position C4, R₂ is OH, and R₃ is 1,1-dimethylpentyl;
n is 2, ring A is unsaturated with a double bond positioned between C3 and C4, R₁ is hydrogen, R₂ is OH, and R₃ is 1,1-dimethylpentyl or 1,1-dimethylheptyl;
n is 2, ring A is saturated, R₁ is hydrogen or gem-dimethyl at position C4, R₂ is OCH₂C(O)OH, and R₃ is 1,1-dimethylheptyl or 1,1-dimethylheptyl;
n is 2, ring A is saturated, R₁ is hydrogen, R₂ is OH, and R₃ is selected from the group consisting of 2-methyl-[1,3]dithiolan-2-yl, C(O)CH₃ and C(O)OCH₃;
n is 2, ring A is saturated, R₁ is hydrogen, R₂ is selected from the group consisting of OCH₂CH(OH)CH₂OH, OC(O)CH═CHC(O)OH, OC(O)CH₃, OC(O)-piperidine, OCH₂-tetrazole, OP(O)(OC₂H₅)₂, OP(O)(OH)₂, OC(O)(CH₂)₃Br and OC(O)(CH₂)₃ONO₂, and R₃ is 1,1-dimethylpentyl;
n is 2, ring A is saturated, R₁ is carbonyl or oxime at position C3 with or without a further gem-dimethyl at position C4, R₂ is OCH₂SCH₃, and R₃ is 1,1-dimethylpentyl;
n is 2, ring A is saturated, R₁ is gem-dimethyl at position C4, R₂ is OC(O)CH═CHC(O)OH or OC(O)(CH₂)₂NHCH₃, and R₃ is 1,1-dimethylheptyl;
n is 2, ring A is saturated, R₁ is gem-dimethyl at position C4, R₂ is OH or OC(O)CH═CHC(O)OH, and R₃ is 2-phenethyl-[1,3]dithiolan-2-yl;
n is 2, ring A is saturated, R₁ is OH at position C3, R₂ is OC(O)CH₂—O—(CH₂)₂—O—(CH₂)₂OCH₃, and R₃ is 1,1-dimethylheptyl;
n is 2, ring A is saturated, R₁ is hydrogen, R₂ is OH, and R₃ is 1,1-dimethylpentyl and R₄ is NO₂ either at ortho, para, or both ortho and para position to R₂;
n is 2, ring A is unsaturated with a double bond positioned between C1 and C2, R₁ is hydrogen, R₂ is OH or OC(O)CH═CHC(O)OH, and R₃ is 1,1-dimethylpentyl or 1,1-dimethylheptyl;
n is 2, ring A is unsaturated with a double bond positioned between C1 and C2, R₁ is a carbonyl at position C3 and a gem-dimethyl at position C6, R₂ is OH or OCH₃, and R₃ is 1,1-dimethylheptyl;
n is 2, ring A is unsaturated with a double bond positioned between C1 and C2, R₁ is a carbonyl at position C3 and a gem-dimethyl at position C5, R₂ is OCH₃, and R₃ is 1,1-dimethylheptyl;
n is 2, ring A is unsaturated with a double bond positioned between C1 and C2, R₁ is a gem-dimethyl at position C4, R₂ is OH, and R₃ is 1,1-dimethylheptyl;
n is 3, ring A is saturated, R₁ is selected from the group consisting of hydrogen, OH, and carbonyl at position C3, R₂ is OH, and R₃ is 1,1-dimethylpentyl or 1,1-dimethylheptyl;
n is 3, ring A is saturated, R₁ is carbonyl at position C3 or hydroxyl at both position C3 and C4, R₂ is OH, and R₃ is 1,1-dimethylpentyl;
n is 3, ring A is saturated, R₁ is hydrogen, R₂ is OCH₂CH₂-morpholine, and R₃ is 1,1-dimethylpentyl or 1,1-dimethylheptyl;
n is 3, ring A is saturated, R₁ is hydrogen, R₂ is OCH₂C(O)OH or OC(O)CH═CHC(O)OH, and R₃ is 1,1-dimethylheptyl;
n is 3, ring A is saturated, R₁ is OH at position C3, R₂ is selected from the group consisting of OCH₂C(O)OH, OP(O)(OH)₂, O(CH₂)₃C(O)OH, OCH₂C(O)N(C₂H₅)₂, O(CH₂)₂—-morpholine and OCH₂-tetrazole, and R₃ is 1,1-dimethylheptyl;
n is 3, ring A is saturated, R₁ is OC(O)CH═CHC(O)OH or iodine at position C3, R₂ is OC(O)CH═CHC(O)OH, and R₃ is 1,1-dimethylheptyl;
n is 3, ring A is saturated, R₁ is hydrogen or OH at position C3, R₂ is OH, and R₃ is pentyl;
n is 3, ring A is saturated, R₁ is selected from the group consisting of oxime, iodine or NH₂ at position C3, C(O)OCH₃, CH₂OH, CH₂C(O)OCH₃ or C(O)OH at position C7, and both OH at position C3 and C(O)OH at position C7, R₂ is OH, and R₃ is 1,1-dimethylheptyl;
n is 3, ring A is saturated, R₁ is NH₂ at position C3, R₂ is H, and R₃ is 1,1-dimethylheptyl;
n is 3, ring A is unsaturated between C3 and C4, R₁ is hydrogen, R₂ is OH, and R₃ is 1,1-dimethylheptyl;
n is 3, ring A is saturated, R₁ is OH at position C3, R₂ is OH, R₃ is 1,1-dimethylheptyl and R₄ is NO₂ either at ortho or para position to R₂.

The compounds of the invention can be prepared by synthetic methods that may produce not only stereoisomers, but also regioisomers which are structural isomers of each other. Thus compounds of formula (I) are regioisomers of compounds of formula (II) and all regioisomers are intended to be included within the scope of the present invention.

According to another aspect, the present invention provides a compound of formula (II):

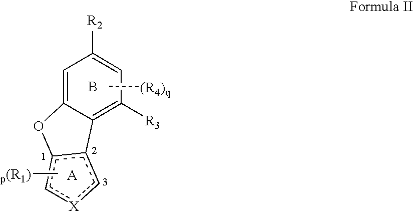

wherein [symbol]
represents a single or double bond;
X, R₁ through R₄ and m, n, p and q are as defined in formula (I);
and stereoisomers, pharmaceutically acceptable salts, esters, polymorphs or solvates of said compounds;
with the provisos that (a) A is not a phenyl ring; and (b) when n is 2, and R₁ at C2 is isopropyl then R₁ at C5 is other than methyl.

According to certain embodiments, the present invention provides a compound of formula (II) as defined therein, wherein n is an integer from 1 to 3, ring A is unsaturated, R₁ is selected from the group consisting of hydrogen, carbonyl, and R, R₂ is OR, and R₃ is a saturated or unsaturated, linear, branched or cyclic C₁-C₁₂ alkyl wherein R is as previously defined.

According to additional embodiments, the present invention provides a compound of formula (II) wherein n is 2, ring A is unsaturated and the double bond is positioned between C1 and C2, R₁ is hydrogen, carbonyl or CH₃, R₂ is OCH₃ and R₃ is 1,1-dimethylheptyl.

According to exemplary embodiments, the present invention provides a compound of formula (II) wherein n is 2, ring A is unsaturated and the double bond is positioned between C1 and C2, R₁ is a carbonyl at position C6 and a gem-dimethyl at position C3 or C4, R₂ is OCH₃ and R₃ is 1,1-dimethylheptyl.

It is understood that the present invention specifically excludes known compounds, including CBF, CBE and the benzofuran derivatives disclosed by Cardillo, in patents Nos. DE 199 34 952, EP 1206934, U.S. Pat. No. 4,960,815, and in international patent applications Nos. WO 00/08007, WO 00/07579 and WO 03/045375; though certain novel properties of these compounds are contemplated within the scope of the present invention.

The compounds of the invention can be used for the preparation of a medicament either as the active ingredient, as is, or in the form of their pharmaceutically acceptable salts, esters, polymorphs, solvates and derivatives.

According to a further aspect, the present invention provides a pharmaceutical composition comprising a prophylactically and/or therapeutically effective amount of a compound of formula (I):

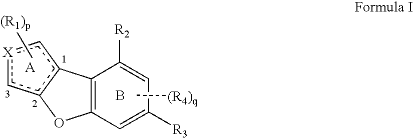

and stereoisomers, pharmaceutically acceptable salts, esters, polymorphs or solvates of said compounds, wherein [symbol]
represents a single or double bond;
X, R₁ through R₄ and m, n, p and q are as defined above in formula (I) with the provisos defined therein;
and further comprising a pharmaceutically acceptable diluent or carrier.

According to certain embodiments, the present invention provides a pharmaceutical composition comprising as an active ingredient a compound of formula (I) as defined therein, wherein the exemplary substituents X and R₁ through R₄ are as defined for formula (I).

According to a further aspect, the present invention provides a pharmaceutical composition comprising a prophylactically and/or therapeutically effective amount of a compound of formula (II):

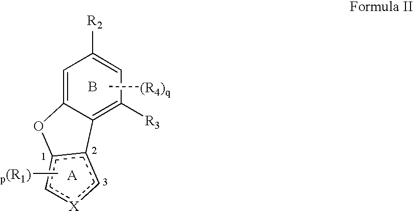

and stereoisomers, pharmaceutically acceptable salts, esters, polymorphs or solvates of said compounds, wherein [symbol]
represents a single or double bond;
X, R₁ through R₄ and m, n, p and q are as defined in formula (I); and further comprising a pharmaceutically acceptable diluent or carrier; with the provisos that (a) A is not a phenyl ring; and (b) when n is 2, and R₁ at C2 is isopropyl then R₁ at C5 is other than methyl.

According to certain embodiments, the present invention provides a pharmaceutical composition comprising as an active ingredient a compound of formula (II) as defined therein, wherein the exemplary substituents X and R₁ through R₄ are as defined for formula (II).

Pharmaceutical compositions of the present invention can include in addition to the aforesaid compounds, pharmaceutically inert ingredients such as thickeners, carriers, buffers, diluents, surface active agents, preservatives and the like, all as well known in the art, necessary to produce physiologically acceptable and stable formulations.

The choice of the pharmaceutical additives, carriers, diluents, excipients and the like, will be determined in part by the particular active ingredient, as well as by the particular route of administration of the composition. The routes of administration include but are not limited to oral, aerosol, parenteral, topical, ocular, transdermal, subcutaneous, intravenous, intramuscular, intraperitoneal, intrathecal, rectal and vaginal.

The pharmaceutical compositions can be in a liquid, aerosol or solid dosage form, and can be formulated into any suitable formulation including, but not limited to, solutions, suspensions, micelles, emulsions, microemulsions, aerosols, powders, granules, sachets, soft gels, capsules, tablets, pills, caplets, suppositories, creams, gels, pastes, foams and the like, as will be required by the particular route of administration.

The present invention provides use of compounds of the general formula (I) or (II) for the preparation of a medicament for preventing, alleviating or treating inflammation, autoimmune diseases, pain, neurological disorders, neurodegenerative diseases, neuroinflammatory conditions, ocular disorders, bone disorders, cardiovascular and cardio-inflammatory disorders, appetite disorders, emetic conditions and certain types of cancer.

The anti-inflammatory and immunomodulatory activities of compounds of the invention will be useful for preventing, alleviating or treating inflammation and inflammatory conditions including but not limited to inflammatory bowel disease, Crohn's disease, ulcerative colitis, autoimmune diseases, allergies and allergic reactions, rheumatoid arthritis, juvenile arthritis, osteoarthritis, multiple sclerosis, systemic lupus erythematosis, myasthenia gravis, diabetes mellitus type I, hepatitis, psoriasis, immune related disorders including but not limited to tissue rejection in organ transplants, malabsorption syndromes such as celiac disease, pulmonary diseases such as asthma, chronic bronchitis, chronic obstructive pulmonary disease (COPD) and Sjögren's syndrome.

The analgesic activities of compounds of the invention will be useful for preventing, alleviating or treating pain including but not limited to peripheral, visceral, neuropathic, inflammatory and referred pain.

The neuroprotective activities of compounds of the invention will be useful for preventing, alleviating or treating neurological disorders, neurodegenerative diseases and neuroinflammatory conditions including but not limited to stroke, migraine, cluster headache, epilepsy, Parkinson's disease, Alzheimer's disease, amyotrophic lateral sclerosis, Huntington's chorea, prion-associated diseases, poisoning of the central nervous system, motor disorders, muscle spasm and tremor, meningitis, encephalitis, cerebral ischemia, and Guillain-Barré syndrome.

The cardioprotective activities of compounds of the invention will be useful for preventing, alleviating or treating cardiovascular and cardio-inflammatory disorders including but not limited to atherosclerosis, pericarditis, myocarditis, endocarditis, arrhythmia, hypertension and myocardial ischemia.

The anti-neoplastic activities of compounds of the invention will be useful for preventing, alleviating or treating certain types of cancer including but not limited to malignant brain tumors, skin tumors, lung adenocarcinoma, uterus, breast and prostate carcinoma, lymphoma, glioma, thyroid epithelioma, and neuroblastoma.

The compounds of the invention will be useful for preventing, alleviating or treating bone disorders including abnormal bone metabolism, Paget's disease, and osteoporosis, ocular disorders including glaucoma, appetite disorders including anorexia and cachexia, and emetic conditions including vomiting and nausea.

In addition, the present invention provides methods of preventing, alleviating or treating aforesaid conditions which comprises administering to a subject in need thereof a prophylactically and/or therapeutically effective amount of a compound of formula (I) or (II) as defined above, or a pharmaceutical composition comprising said compound.

These and additional benefits and features of the invention could be better understood by those skilled in the art with reference to the following detailed description taken in conjunction with the figures and non-limiting examples.

### BRIEF DESCRIPTION OF THE FIGURES

The accompanying drawings, which are incorporated in and form a part of the specification, illustrate certain embodiments of the present invention, and together with the description serve to explain the principles of the invention. In the drawings:

FIG. 1 shows in tabulated form the chemical structures of certain compounds of the invention, together with some physicochemical and biological information.

FIG. 2 shows the binding affinity, (Panel A) and agonistic activity of an exemplary compound of the invention, C6S-37, toward the human CB₁ and CB₂ cannabinoid receptors (Panel B and C, respectively).

FIG. 3 shows the dose related analgesic activity of compounds of the invention in a model of visceral pain. The number of writhing responses (WR) is plotted for each treatment group.

FIG. 4 shows the anti-inflammatory and analgesic activity of compounds of the invention in a model of inflammatory pain. Panel A shows the anti-inflammatory activity on paw edema as expressed in percent swelling over naïve paw. Panel B shows the analgesic activity following thermal stimuli as expressed in A latency time in seconds. Panel C shows the analgesic activity following mechanical stimuli as expressed in A force in grams.

FIG. 5 shows the analgesic activity of compounds of the invention in a model of chronic pain induced by sciatic nerve ligation. Results are expressed as Δforce in grams following mechanical stimulus at various time points plotted in hours.

FIG. 6 shows the immunomodulatory activity of exemplary compound C7S-2 administered p.o. and i.p. on PLP induced remitting-relapsing EAE.

FIG. 7 shows the anti-inflammatory and gastro-protective activity of compounds of the invention in a model of inflammatory bowel disease.

### DETAILED DESCRIPTION OF THE INVENTION

The present invention provides new benzofuran derivatives, which may be considered as non-classical cannabinoids, pharmaceutical compositions comprising the same and methods of use thereof.

Many classes of cannabinoids were identified in nature, including for example the classical THC type and the non-classical endocannabinoids [Di Marzo V. et al., Nature Reviews Drug Discovery 3(9): 771-84, 2004]. In the past decades, many more chemical families were designed as synthetic cannabinoid analogues and they include aminoalkyl indoles such as WIN 55, 212-2, pinene derivatives such as HU-308, pyrazoles such as SR 141716A, imidazoles, thiazoles, tetrahydroquinolines, heteroindanes and substituted sulfonamides for example.

Generally, the activity of cannabinoids is mediated by agonistic or antagonistic interactions with membrane-bound cannabinoid receptors. But evidence exists pointing to the existence of yet unidentified sites of action independent of known cannabinoid receptors. These alternative mechanisms include for example non-cannabinoid receptor mediated activity and intrinsic properties. The synthetic THC type cannabinoid dexanabinol, for instance, which does not bind to CB₁ and CB₂ receptors, was shown to act as a NMDA antagonist and to display antioxidant properties.

The new compounds of the invention are benzofuran derivatives and they can be considered to belong to an additional class of cannabinoids. Similarly to more traditional cannabinoids, these compounds can act through agonistic or antagonistic modulation of cannabinoid receptors and/or through non-cannabinoid receptor or non-receptor mediated mechanisms.

The compounds of the invention can possess one or more chiral centers, and can therefore be produced as individual stereoisomers such as enantiomers (mirror images) and diastereomers (not mirror images) or as mixtures, racemic or otherwise, of stereoisomers, depending on synthetic conditions and appropriate separation and isolation. Mixtures of enantiomers and diastereomers can be separated into stereoisomerically uniform components in a known manner or synthesized a priori as separate enantiomers or diastereomers. All of these individual stereoisomers and mixture thereof are intended to be included within the scope of the present invention

The compounds of the invention can be prepared by synthetic methods that may produce not only stereoisomers, but also regioisomers. Regioisomers are structural isomers that can potentially arise from the same reaction or that can be prepared individually under regioselective reaction conditions. Thus compounds of formula (I) are regioisomers of compounds of formula (II) and all regioisomers are intended to be included within the scope of the present invention.

### DEFINITIONS

To facilitate an understanding of the present invention, a number of terms and phrases are defined below.

As used herein, the term “central nervous system” (CNS) refers to all structures within the dura mater. Such structures include, but are not limited to, the brain and spinal cord.

As used herein, the term “CB” refers to cannabinoid receptors. CB₁ receptors are predominantly found in the CNS, whereas CB₂ receptors are predominantly found in the periphery on immune cells. hCB₁ and hCB₂ indicate that the receptors are of human origin. Aside from these two receptors, evidence exists supporting the presence of yet uncloned cannabinoid receptors.

As used herein, the term “cannabinoid” or “cannabinoids” refers to natural, plant derived or endogenous, or synthetic compounds, metabolites and analogues thereof, whose effects are generally mediated by cannabinoid receptors, but can also act through other receptors or through receptor independent mechanisms.

In the present invention, binding affinity is represented as indicated either by the dissociation constant Kᵢ, which represents the concentration of the unlabelled drug that will bind to half the binding sites at equilibrium in the absence of radioligand or as percent displacement at a given compound concentration, when a full dose range curve was not yet established. The Kᵢ value is calculated based on the IC₅₀ value of the test compound, namely the concentration of a test compound that will displace 50% of a radiolabeled agonist from the CB receptors, the radioligand concentration and its dissociation constant K_{d}. Compounds specific for a given receptor display Kᵢ value for binding of said receptor of 50 nM or lower, preferably of 30 nM or lower, more preferably of 10 nM or lower and most preferably of 1 nM or lower. Compounds selective for a given receptor display a ratio of binding affinity between the receptors under consideration of at least 5, preferably 10, more preferably 20 and most preferably 50 or greater. Preferably these ratios will be obtained for human CB₁ and CB₂ receptors. Compounds of the present invention may or may not exhibit binding affinity toward each cannabinoid receptor, as well as may or may not display selectivity toward one of the receptors.

An agonist is a substance that mimics a specific ligand, for example a hormone, a neurotransmitter, or in the present case a cannabinoid, able to attach to that ligand's receptor and thereby produce the same action that the natural ligand produces. Though most agonists act through direct binding to the relevant receptor and subsequent activation, some agonists act by promoting the binding of the ligand or increasing its time of residence on the receptor, increasing the probability and effect of each coupling. Compounds that have the opposite effect, and instead of promoting the action of a ligand, block it, are receptor antagonists. The novel benzofuran derivatives described herein that interact with at least one cannabinoid receptor can initiate either an agonistic or an antagonistic response from said receptor, and both mechanisms of action are encompassed in the present inventions.

Though the most probable mechanism of action of the compounds of the invention is through their binding to the known cannabinoid receptors and functional coupling to or blocking of specific signal transduction pathways, alternative mechanisms cannot be ruled out, for instance either through binding to additional yet unidentified cannabinoid receptors or through non-cannabinoid receptor or non-receptor mediated means, or a combination of such mechanisms.

In the present specification and claims which follow “inhibiting, reducing, or decreasing effect” means the ability to reduce the activity under discussion by at least 20%, preferably 40%, more preferably 60% and most preferably 80% or greater. In case of activities wherein the maximal possible effect is not 100%, the previous figures relate to percent of maximal possible effect.

In the present specification and claims which follow “enhancing or increasing effect” means the ability to increase the activity under discussion by at least about 1.5 fold, preferably about 3 folds, more preferably about 4 folds and most preferably above 5 folds or more.

### Chemical Definitions

In the present invention, the positions in the A ring structure will be numbered clockwise, wherein positions 1, 2, and 3 are as shown in formulae (I) and (II). Ring A, which may consist of 4 to 8 carbon atoms, may comprise one or more double bonds at any position on the ring, wherein two double bonds may not be adjacent to each other.

The alkyl substituents can be saturated or unsaturated (e.g. alkenyl, alkynyl), linear, branched or cyclic, the latter only when the number of carbon atoms in the alkyl chain is greater than or equal to three, and can contain mixed structures. When unsaturated, the hydrocarbon radicals can have one double bond or more and form alkenyls, or one triple bond or more and form alkynyls. Regardless of the degree of unsaturation, all of the alkyl substituents can be linear or branched.

OR represents hydroxyl or ethers, OC(O)R and C(O)OR represent esters, OC(O)OR represent carbonate esters, C(O)R represents ketones, OC(O)NR₂ represents, carbamates, NR₂ represents amines, C(O)NR₂ represents amides, SR represents thiols or sulfides, S(O)R represents sulfoxides, S(O)(O)R represents sulfones, P(O)(OR)₂ represents phosphates, OP(O)(OR)₂ represents ester phosphates, when R is a hydrogen or an alkyl chain.

“Gem-dimethyl” means that two methyl groups are attached on the same carbon atom.

“Halogen” or “halo” means fluorine (—F), chlorine (—Cl), bromine (—Br) or iodine (—I) and if the compound contains more than one halogen (e.g., two or more variable groups can be a halogen), each halogen is independently selected from the aforementioned halogen atoms.

The term “heterocyclic ring” means a stable unsubstituted or substituted, saturated or unsaturated ring system of up to 6 atoms which consists of carbon atoms and at least one heteroatom selected from the group consisting of N, O, and S. The nitrogen and sulfur heteroatoms can be optionally oxidized, and the nitrogen atom can be optionally quaternized. The heterocyclic system can be attached, unless otherwise stated, at any heteroatom or carbon atom which affords a stable structure. Heterocyclic rings include for example: furan, thiazole, triazole, tetrazole, pyrolle, pyrrolidine, pyrazole, imidazole, pyridine, piperidine, pyrazine, piperazine, pyrimidine, oxadiazole, succinimide, morpholine and thiomorpholine.

The term “aryl” refers to an aromatic cyclic hydrocarbon group of from 6 to 20 carbon atoms having a single ring (e.g., phenyl) or multiple condensed (fused) rings (e.g., naphthyl or antliryl). Preferred aryls include phenyl, naphthyl and the like. The term aryl includes both “unsubstituted aryls” and “substituted aryls”, the latter of which refers to aryl moieties having substituents replacing a hydrogen on one or more carbons of the ring. Such substituents can include, but are not limited to hydroxy, alkoxy, alkyl, alkenyl, nitro, carboxy, carbonyl, amino, or halogen.

It is to be understood that the present invention covers all combinations of particular and preferred groups mentioned hereinabove.

The term “substituted” or “optionally substituted” means that one or more hydrogens on the designated atom is replaced or optionally replaced with a selection from the indicated group, provided that the designated atom's normal valency under the existing circumstances is not exceeded. Combination of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The present invention also includes within its scope solvates of compounds of formulae (I) and (II) and salts thereof. “Solvate” means a physical association of a compound of the invention with one or more solvent molecules. This physical association involves varying degrees of ionic bonding, including hydrogen bonding. In certain instances the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

The term “polymorph” refers to a particular crystalline state of a substance, which can be characterized by particular physical properties such as X-ray diffraction, IR spectra, melting point, and the like.

In the present specification the term “prodrug” represents compounds which are rapidly transformed in vivo to parent compound of formulae (I) and (II), for example by hydrolysis in the blood. Prodrugs are often useful because in some instances they can be easier to administer than the parent drug. They can, for instance, be bioavailable by oral administration whereas the parent drug is not. The prodrug can also have improved solubility compared to the parent drug in pharmaceutical compositions. All of these pharmaceutical forms are intended to be included within the scope of the present invention.

Certain compounds of the invention are capable of further forming pharmaceutically acceptable salts and esters. “Pharmaceutically acceptable salts and esters” means any salt and ester that is pharmaceutically acceptable and has the desired pharmacological properties. Such salts, formed for instance by any carboxy or sulfo groups present in the molecule, include salts that can be derived from an inorganic or organic acid, or an inorganic or organic base, including amino acids, which is not toxic or otherwise unacceptable.

Pharmaceutically acceptable acid addition salts of the compounds include salts derived from inorganic acids such as hydrochloric, nitric, phosphoric, sulfuric, hydrobromic, hydriodic, phosphorous, and the like, as well as salts derived from organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate or galacturonate [Berge S. M. et al., J. of Pharmaceutical Science, 66: 1-19, 1977].

The acid addition salts of said basic compounds are prepared by contacting the free base form with a sufficient amount of the desired acid to produce the salt in the conventional manner. The free base form can be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

The base addition salts of said acidic compounds are prepared by contacting the free acid form with a sufficient amount of the desired base to produce the salt in the conventional manner. The free acid form can be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

### Pharmacology

In the present specification and claims which follow the compositions comprising an effective amount of a compound are intended to encompass both prophylactically and therapeutically effective compositions.

The term “prophylactically effective” refers to the amount of compound which will achieve the goal of prevention, reduction or eradication of the risk of occurrence of the disease or disorder, while avoiding adverse side effects. The term “therapeutically effective” refers to the amount of compound that will achieve, with no adverse effects, alleviation, diminished progression or treatment of the disorder, once the disorder cannot be further delayed and the patients are no longer asymptomatic, hence providing either a subjective relief of a symptom (s) or an objectively identifiable improvement as noted by the clinician or other qualified observer.

The “subject” or “patient” for purposes of treatment includes any human or animal affected by any of the diseases where the treatment has beneficial therapeutic impact. Usually, the animal that serves to establish the pre-clinical data and that can be treated by compounds of the invention is a vertebrate such as a primate including chimpanzees, monkeys and macaques, a rodent including mice, rats, ferrets, rabbits and hamsters, a domestic or game animal including bovine species, equine species, pigs, sheeps, caprine species, feline species, canine species, avian species, and fishes

By virtue of their shared properties with other classes of cannabinoids, it will be recognized that the compositions according to the present invention will be useful for preventing, alleviating or treating indications amenable to cannabinoid intervention exemplified by pain, inflammation, immune, neurological, ocular, bone, cardiovascular and motor disorders, appetite stimulation, emesis, nausea, glaucoma and certain types of cancer. A detailed list of pathological states wherein administration of cannabinoids can be useful can be found in international patent application No. WO 2004/018433. The book of Grotenhermen F. and Russo E., “Cannabis and cannabinoids. Pharmacology, toxicology and therapeutic potential”, published by Hatworth Press in 2002, provides a comprehensive source for the various conditions and diseases, reviewed in Chapter 11 therein, where cannabinoids have recognized therapeutic potential.

By virtue of their anti-inflammatory and immunomodulatory properties, it will be recognized that the compositions according to the present invention will be useful for preventing, alleviating or treating indications having an inflammatory or autoimmune mechanism involved in their etiology or pathogenesis exemplified by arthritis, including rheumatoid arthritis, juvenile arthritis, osteoarthritis, allergies and allergic reactions, multiple sclerosis, systemic lupus erythematosus (SLE), myasthenia gravis, diabetes mellitus type I, hepatitis, psoriasis, immune related disorders including but not limited to tissue rejection in organ transplants, malabsorption syndromes such as celiac, pulmonary diseases such as asthma, chronic bronchitis, chronic obstructive pulmonary disease and Sjögren's syndrome, inflammatory bowel disease, Crohn's disease, ulcerative colitis, and rheumatic diseases. The potential of cannabinoids as anti-inflammatory therapeutics was recently reviewed by Klein [Klein T. W., Nature Reviews Immunology 5: 400-11, 2005].

By virtue of their neuroprotective properties, it will be recognized that the compositions according to the present invention will be useful in treating neurological disorders including but not limited to stroke, migraine, cluster headaches and epilepsy. The compositions of the present invention can also be effective in treating certain chronic degenerative diseases that are characterized by gradual selective neuronal loss, including by promoting neurogenesis. In this connection, the compositions of the present invention are contemplated as therapeutically effective in the treatment of Parkinson's disease, Alzheimer's disease, amyotrophic lateral sclerosis, Huntington's chorea, motor disorders including spasm and tremor, and prion-associated neurodegeneration. Other therapeutic targets for compositions of the invention having a neuroinflammatory basis include for example meningitis, encephalitis, cerebral ischemia, and Guillain-Barré syndrome. Neuroprotection could also be effective in protection and/or treatment of neurotoxic agents, such as nerve gas, as well as other insults to brain or nervous tissue by way of chemical or biological agents.

By virtue of their analgesic properties it will be recognized that the compositions according to the present invention will be useful in treating pain including peripheral, visceral, neuropathic, inflammatory and referred pain. Some of the recent findings concerning the utility of cannabinoids as analgesics, as well as anti-inflammatory agents, was recently reviewed by Mbvundula et al. [Mbvundula E. C. et al., Inflammo-pharmacology 12(2): 99-114, 2004].

Another feature of the present invention is the ability of the disclosed compounds to prevent or treat certain cancers, including malignant brain tumors, skin tumors, lung adenocarcinoma, uterus, breast and prostate carcinoma, lymphoma, glioma, thyroid epithelioma, and neuroblastoma, where CB ligands can trigger apoptosis of tumor cells as well as inhibiting tumor angiogenesis. The potential of cannabinoids as anti-cancer agents was recently reviewed by Guzman [Guzman M., Nature Reviews Cancer 3: 745-55, 2003]. As used herein, the term “cancer” includes both solid and non-solid tumors, as well as cancer metastasis.

The therapeutic potential of cannabinoids in cardiovascular diseases was recently reviewed by Pacher et al. [Pacher P. et al., Handb. Exp. Pharmacol. 168: 599-625, 2005] and their role in atherosclerosis, a disease having important inflammatory and immune components, was addressed by Steffens et al. [Steffens S. et al., Nature 347: 782-6, 2005]. The anti-inflammatory activity of compounds of the invention, when applied to the cardiovascular system, makes compositions of the invention also useful for the treatment of pericarditis, myocarditis and endocarditis.

Both the CB₁ and CB₂ receptors seem to be involved in the pathogenesis of osteoporosis and other bone diseases [Idris A. I. et al., Nature Medicine 11(7): 774-9, 2005; Ofek O. et al., PNAS 103(3): 696-701, 2006]. Hence, it will be recognized that the compositions according to the present invention will be useful in treating bone disorders including abnormal bone metabolism, Paget's disease and osteoporosis.

Hereinafter, the term “oral administration” includes, but is not limited to, administration by mouth for absorption through the gastrointestinal tract (peroral) wherein the drug is swallowed, or for trans-mucosal absorption in the oral cavity by buccal, gingival, lingual, sublingual and oro-pharyngeal administration. Compositions for oral administration include powders or granules, suspensions or solutions in water or non-aqueous media, sachets, capsules or tablets. The oral composition can optionally contain inert pharmaceutical excipients such as thickeners, diluents, flavorings, dispersing aids, emulsifiers, binders, preservatives and the like.

The term “parenteral administration” indicates any route of administration other than via oral administration and includes, but is not limited to, administration by intravenous drip or bolus injection, intraperitoneal, intrathecal, intralesional, subcutaneous, or intra muscular injection, topical, ocular, transdermal, rectal, vaginal, nasal administration or by inhalation.

Formulations for parenteral administration include but are not limited to sterile aqueous solutions which can also contain buffers, diluents and other suitable additives.

The compositions described herein are also suitable for administration in immediate release formulations, and/or in controlled or sustained release formulations. The sustained release systems can be tailored for administration according to any one of the proposed administration regimes. Slow or extended-release delivery systems, including any of a number of biopolymers (biological-based systems), systems employing liposomes, and polymeric delivery systems, can be utilized with the compositions described herein to provide a continuous or long term source of therapeutic compound(s).

It is to be understood that the phraseology or terminology used herein is for the purpose of description and not of limitation, such that the terminology or phraseology of the present specification is to be interpreted by the skilled artisan in light of the teachings and guidance presented herein, in combination with the knowledge of one of ordinary skill in the art.

The pharmaceutical compositions can contain in addition to the active ingredient conventional pharmaceutically acceptable carriers, diluents and excipients necessary to produce a physiologically acceptable and stable formulation. The terms carrier, diluent or excipient mean an ingredient that is compatible with the other ingredients of the compositions disclosed herein, especially substances which do not react with the compounds of the invention and are not overly deleterious to the patient or animal to which the formulation is to be administered. For compounds having poor solubility, and for some compounds of the present invention that are characteristically hydrophobic and practically insoluble in water with high lipophilicity, as expressed by their high octanol/water partition coefficient and log P values, formulation strategies to prepare acceptable dosage forms will be applied. Enabling therapeutically effective and convenient administration of the compounds of the present invention is an integral part of this invention.

The pharmaceutical compositions can be in a liquid, aerosol or solid dosage form, and can be formulated into any suitable formulation including, but not limited to, solutions, suspensions, micelles, emulsions, microemulsions, aerosols, ointments, gels, suppositories, capsules, tablets, and the like, as will be required for the appropriate route of administration.

Solid compositions for oral administration such as tablets, pills, capsules, softgels or the like can be prepared by mixing the active ingredient with conventional, pharmaceutically acceptable ingredients such as corn starch, lactose, sucrose, mannitol, sorbitol, talc, polyvinylpyrrolidone, polyethyleneglycol, cyclodextrins, dextrans, glycerol, polyglycolized glycerides, tocopheryl polyethyleneglycol succinate, sodium lauryl sulfate, polyethoxylated castor oils, non-ionic surfactants, stearic acid, magnesium stearate, dicalcium phosphate and gums as pharmaceutically acceptable diluents. The tablets or pills can be coated or otherwise compounded with pharmaceutically acceptable materials known in the art, such as microcrystalline cellulose and cellulose derivatives such as hydroxypropylmethylcellulose (HPMC), to provide a dosage form affording prolonged action or sustained release. Coating formulations can be chosen to provide controlled or sustained release of the drug, as is known in the art.

Other solid compositions can be prepared such as suppositories or retention enemas, for rectal administration using conventional suppository bases such as cocoa butter or other glycerides. Liquid forms can be prepared for oral administration or for injection, the term including but not limited to subcutaneous, transdermal, intravenous, intrathecal, intralesional, adjacent to or into tumors, and other parenteral routes of administration. The liquid compositions include aqueous solutions, with or without organic cosolvents, aqueous or oil suspensions including but not limited to cyclodextrins as suspending agent, flavored emulsions with edible oils, triglycerides and phospholipids, as well as elixirs and similar pharmaceutical vehicles. In addition, the compositions of the present invention can be formed as aerosols, for intranasal and like administration. For administration by inhalation, the compounds of the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch. Topical pharmaceutical compositions of the present invention can be formulated as solution, lotion, gel, cream, ointment, emulsion or adhesive film with pharmaceutically acceptable excipients including but not limited to propylene glycol, phospholipids, mono glycerides, diglycerides, triglycerides, polysorbates, surfactants, hydrogels, petrolatum or other such excipients as are known in the art.

Pharmaceutical compositions of the present invention can be manufactured by processes well known in the art, e.g., by means of conventional mixing, dissolving, wet granulating, dry-mixing, direct compression, grinding, pulverizing, dragee-making, levigating, emulsifying, encapsulating, entrapping or lyophilizing processes.

Prior to their use as medicaments, the pharmaceutical compositions can be formulated in unit dosage forms. The active dose for humans can be determined by standard clinical techniques and is generally in the range of from 0.01 mg to about 50 mg per kg body weight, in a regimen of 1-4 times a day. The preferred range of dosage varies with the specific compound used and is generally in the range of from 0.1 mg to about 20 mg per kg body weight. However, it is, evident to one skilled in the art that dosages would be determined by the attending physician, according to the disease or disorder to be treated, its severity, the desired, therapeutic effect, the duration of treatment, the method and frequency of administration, the patient's age, weight, gender and medical condition, concurrent treatment, if any, i.e., co-administration and combination with additional medications, contraindications, the route of administration, and the like. The administration of the compositions of the present invention to a subject in need thereof can be continuous, for example once, twice or thrice daily, or intermittent for example once weekly, twice weekly, once monthly and the like, and can be gradual or continuous, constant or at a controlled rate.

Effective doses can be extrapolated from dose-response curves derived from in vitro or animal model test systems. For example, an estimated effective mg/kg dose for humans can be obtained based on data generated from mice or rat studies, for an initial approximation the effective mg/kg dosage in mice or rats is divided by twelve or six, respectively.

According to a first aspect, the present invention provides a compound of formula (I):

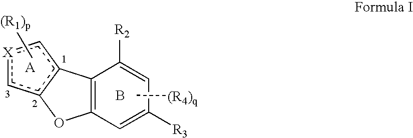

and stereoisomers, pharmaceutically acceptable salts, esters, polymorphs or solvates of said compounds; wherein [symbol]
represents a single or double bond;
X is (CHₘ)ₙ wherein m is an integer from, to 2 and n is an integer from 0 to 4;
R₁ is at each occurrence selected independently from the group consisting of:
a) a halogen;
b) a carbonyl;
c) an aryl;
d) Rₐ wherein Rₐ is selected from the group consisting of R_{b}, OR_{b}, C(O)OR_{b} and OC(O)R_{b} wherein R_{b} is a saturated or unsaturated, linear or branched C₁-C₈ alkyl substituted with one or more heteroatoms selected from the group consisting of N, O and S;
e) R_{c} wherein R_{c} is selected from R, OR, OC(O)OR, C(O)OR, OC(O)R and OC(O)N(R′)₂, wherein R is selected from the group consisting of a hydrogen, a saturated or unsaturated, linear, branched or cyclic C₁-C₆ alkyl, C₁-C₆ alkyl-OR′, C₁-C₆ alkyl-(OR′)₂, C₁-C₆ alkyl-C(O)OR′, and C₁-C₆ alkyl-C(O)N(R′)₂, and wherein R′ is at each occurrence independently selected from the group consisting of a hydrogen and a saturated or unsaturated, linear, branched or cyclic C₁-C₆ alkyl;
f) an oxime; and
g) N(R′)₂, wherein R′ is at each occurrence as previously defined;
p is an integer from 0 to 14;
   R₂ is selected from the group consisting of:
c) a hydrogen;
d) Rₐ or R_{c}, wherein Rₐ and Rₑ are as previously defined; and
c) OR″Z, wherein R″ is selected from the group consisting of a direct bond, C(O), Rₑ and C(O)Rₑ wherein Rₑ is a saturated or unsaturated, linear or branched C₁-C₈ alkyl, and Z is selected from the group consisting of ONO₂, a halogen, P(O)(OR′)₂, SR′, S(O)R′, S(O)(O)R′, N(R′)₂, wherein R′ is as previously defined, and a saturated or unsaturated heterocyclic ring of up to 6 atoms containing at least one heteroatom selected from the group consisting of N, O and S;
   R₃ is selected from the group consisting of:
a) R_{d} wherein R_{d} is selected from the group consisting of hydrogen, C(O)OR′″, C(O)R′″, CN and NO₂, wherein R′″ is selected from the group consisting of a hydrogen and a saturated or unsaturated, linear, branched or cyclic C₁-C₁₂ alkyl;
b) a saturated or unsaturated, linear, branched or cyclic C₂-C₁₂ alkyl which is unsubstituted or substituted by a saturated or unsaturated heterocyclic ring as previously defined;
c) a saturated or unsaturated, linear or branched C₁-C₁₂ alkyl substituted by an aryl; and
d) a saturated or unsaturated heterocyclic ring as previously defined, said ring being unsubstituted or substituted by at least one saturated or unsaturated, linear branched or cyclic C₁-C₆ alkyl, wherein said alkyl can be unsubstituted or substituted by an aryl; and
   R₄ is selected independently at each occurrence from the group consisting of hydrogen, NO₂ and NH₂; and q is an integer from 0 to 2;
   with the provisos that (a) A is not a phenyl ring; (b) when n is 1, R₁ is not a phenyl at position C2; (c) when n is 2, and R₁ at C2 is isopropyl then R₁ at C5 is other than methyl; and (d) when n is 2, R₁ is methyl and hydroxyl at C3 and isopropenyl at C6, then R₁ is other than OH, OCH₃ and OC(O)CH₃.

According to certain embodiments, the present invention provides a compound of formula (I) as defined therein, wherein n is an integer from 1 to 3, p is an integer from 0 to 4, q is an integer from 0 to 2, ring A is saturated or unsaturated wherein the optional double bond on ring A is positioned between C1 and C2 or C3 and C4, R₁ is at each occurrence independently selected from the group consisting of hydrogen, halogen, carbonyl, oxime, NH₂, R, C(O)OR, and OR; R₂ is selected from the group consisting of hydrogen, R_{c}, OR, OR″Z, OC(O)R_{b}, OR_{b} and OC(O)R; R₃ is selected from the group consisting of a saturated or unsaturated, linear, branched or cyclic C₁-C₁₂ alkyl which is unsubstituted or substituted by a heterocyclic ring or by an aryl, C(O)R′″ and C(O)OR′″; and R₄ is selected from the group consisting of hydrogen and NO₂, wherein R, R″, R′″, R_{b}, heterocyclic ring and Z are as previously defined.

According to additional embodiments, the present invention provides a compound of formula (I) as defined therein, wherein:

n is 1, ring A is saturated, R₁ is at each occurrence independently selected from the group consisting of hydrogen and CH₃, R₂ is OH or OC(O)CH═CHC(O)OH, and R₃ is selected from the group consisting of 1,1-dimethylpentyl and 1,1-dimethylheptyl;
n is 2, ring A is saturated or unsaturated wherein the optional double bond is positioned between C1 and C2 or C3 and C4, R₁ is at each occurrence independently selected from the group consisting of hydrogen, carbonyl, isopropylidene, oxime, iodine, OH and CH₃, R₂ is selected from the group consisting of OH, OCH₃, OCH₂C(O)OH, OCH₂SCH₃, OP(O)(OH)₂, OP(O)(OC₂H₅)₂, OCH₂-tetrazole, OCH₂CH₂-morpholine, OCH₂CH(OH)CH₂OH, OC(O)CH═CHC(O)OH, OC(O)CH₃, OC(O)(CH₂)₂NHCH₃, OC(O)-piperidine, OC(O)(CH₂)₃Br and OC(O)(CH₂)₃ONO₂, R₃ is selected from the group consisting of 2-phenethyl-[1,3]-dithiolane, 2-methyl-[1,3]dithiolan-2-yl, C(O)CH₃, C(O)OCH₃, 1,1-dimethylpentyl and 1,1-dimethylheptyl and R₄ is selected from the group consisting of hydrogen and NO₂;
n is 3, ring A is saturated or unsaturated wherein the double bond is between C3 and C4, R₁ is selected from the group consisting of hydrogen, iodine, NH₂, OH, OC(O)CH═CHC(O)OH, C(O)OCH₃, C(O)OH, CH₂OH, CH₂C(O)OCH₃, oxime, and carbonyl, R₂ is selected from the group consisting of hydrogen, OH, OCH₂CH₂-morpholine, OCH₂C(O)OH, OC(O)CH═CHC(O)OH, OCH₂-tetrazole, OP(O)(OH)₂, O(CH₂)₃C(O)OH, OCH₂C(O)N(C₂H₅)₂, and OC(O)CH₂OCH₂CH₂OCH₂CH₂OCH₃, and R₃ is selected from the group consisting of pentyl, 1,1-dimethylpentyl and 1,1-dimethylheptyl.

According to exemplary embodiments, the present invention provides a compound of formula (I) wherein:

n is 1, ring A is saturated, R₁ is selected from the group consisting of hydrogen, CH₃ at position C2, and CH₃ at positions C2 and C3, R₂ is OH and R₃ is 1,1-dimethylheptyl;
n is 1, ring A is saturated, R₁ is CH₃ at position C2, R₂ is OH and R₃ is 1,1-dimethylpentyl;
n is 1, ring A is saturated, R₁ is CH₃ at position C2 and C3, R₂ is OC(O)CH═CHC(O)OH and R₃ is 1,1-dimethylheptyl;
n is 2, ring A is saturated, R₁ is selected from the group consisting of hydrogen, OH, carbonyl, iodine or oxime at position C3, gem-dimethyl at position C4, CH₃ at position C2 and isopropylidene at position C5, carbonyl at position C3 and gem-dimethyl at position C4 and both OH at position C3 and gem-dimethyl at position C4, R₂ is OH, and R₃ is 1,1-dimethylheptyl;
n is 2, ring A is saturated, R₁ is selected from the group consisting of hydrogen, OH, carbonyl or oxime at position C3 with or without a further gem-dimethyl at position C4, iodine at position C3, and gem-dimethyl at position C4, R₂ is OH, and R₃ is 1,1-dimethylpentyl;
n is 2, ring A is unsaturated with a double bond positioned between C3 and C4, R₁ is hydrogen, R₂ is OH, and R₃ is 1,1-dimethylpentyl or 1,1-dimethylheptyl;
n is 2, ring A is saturated, R₁ is hydrogen or gem-dimethyl at position C4, R₂ is OCH₂C(O)OH, and R₃ is 1,1-dimethylheptyl or 1,1-dimethylheptyl;
n is 2, ring A is saturated, R₁ is hydrogen, R₂ is OH, and R₃ is selected from the group consisting of 2-methyl-[1,3]dithiolan-2-yl, C(O)CH₃ and C(O)OCH₃;
n is 2, ring A is saturated, R₁ is selected from the group consisting of hydrogen, R₂ is OCH₂CH(OH)CH₂OH, OC(O)CH═CHC(O)OH, OC(O)CH₃, OC(O)-piperidine, OCH₂-tetrazole, OP(O)(OH)₂, OP(O)(OC₂H₅)₂, OC(O)(CH₂)₃Br and OC(O)(CH₂)₃ONO₂, and R₃ is 1,1-dimethylpentyl;
n is 2, ring A is saturated, R₁ is carbonyl or oxime at position C3 with or without a further gem-dimethyl at position C4, R₂ is OCH₂SCH₃, and R₃ is 1,1-dimethylpentyl;
n is 2, ring A is saturated, R₁ is gem-dimethyl at position C4, R₂ is OC(O)CH═CHC(O)OH or OC(O)(CH₂)₂NHCH₃, and R₃ is 1,1-dimethylheptyl;
n is 2, ring A is saturated, R₁ is gem-dimethyl at position C4, R₂ is OH or OC(O)CH═CHC(O)OH, and R₃ is 2-phenethyl-[1,3]dithiolan-2-yl;
n is 2, ring A is saturated, R₁ is OH at position C3, R₂ is OC(O)CH₂—O—(CH₂)₂—O—(CH₂)₂OCH₃, and R₃ is 1,11-dimethylheptyl;
n is 2, ring A is saturated, R₁ is hydrogen, R₂ is OH, and R₃ is 1,1-dimethylpentyl and R₄ is NO₂ either at ortho, para, or both ortho and para position to R₂;
n is 2, ring A is unsaturated with a double bond positioned between C1 and C2, R₁ is hydrogen, R₂ is OH or OC(O)CH═CHC(O)OH, and R₃ is 1,1-dimethylpentyl or 1,1-dimethylheptyl;
n is 2, ring A is unsaturated with a double bond positioned between C1 and C2, R₁ is a carbonyl at position C3 and gem-dimethyl at position C6, R₂ is OH or OCH₃, and R₃ is 1,1-dimethylheptyl;
n is 2, ring A is unsaturated with a double bond positioned between C1 and C2, R₁ is a carbonyl at position C3 and gem-dimethyl at position C5, R₂ is OCH₃, and R₃ is 1,1-dimethylheptyl;
n is 2, ring A is unsaturated with a double bond positioned between C1 and C2, R₁ is a gem-dimethyl at position C4, R₂ is OH, and R₃ is 1,1-dimethylheptyl;
n is 3, ring A is saturated, R₁ is selected from the group consisting of hydrogen, OH, and carbonyl at position C3, R₂ is OH, and R₃ is 1,1-dimethylpentyl or 1,1-dimethylheptyl;
n is 3, ring A is saturated, R₁ is carbonyl at position C3 or hydroxyl at both positions C3 and C4, R₂ is OH, and R₃ is 1,1-dimethylpentyl;
n is 3, ring A is saturated, R₁ is hydrogen, R₂ is OCH₂CH₂-morpholine, and R₃ is 1,1-dimethylpentyl or 1,1-dimethylheptyl;
n is 3, ring A is saturated, R₁ is hydrogen, R₂ is OCH₂C(O)OH or OC(O)CH═CHC(O)OH, and R₃ is 1,1-dimethylheptyl;
n is 3, ring A is saturated, R₁ is OH at position C3, R₂ is selected from the group consisting of OCH₂C(O)OH, OP(O)(OH)₂, O(CH₂)₃C(O)OH, OCH₂C(O)N(C₂H₅)₂, O(CH₂)2-morpholine and OCH₂-tetrazole, and R₃ is 1,1-dimethylheptyl;
n is 3, ring A is saturated, R₁ is iodine or OC(O)CH═CHC(O)OH at position C3, R₂ is OC(O)CH═CHC(O)OH, and R₃ is 1,1-dimethylheptyl;
n is 3, ring A is saturated, R₁ is hydrogen or OH at position C3, R₂ is OH, and R₃ is pentyl;
n is 3, ring A is saturated, R₁ is selected from the group consisting of oxime, iodine, or NH₂ at position C3, C(O)OCH₃, CH₂OH, CH₂C(O)OCH₃ or C(O)OH at position C7, and both OH at position C3 and C(O)OH at position C7, R₂ is OH, and R₃ is 1,1-dimethylheptyl;
n is 3, ring A is saturated, R₁ is NH₂ at position C3, R₂ is H, and R₃ is 1,1-dimethylheptyl;
n is 3, ring A is unsaturated between C3 and C4, R₁ is hydrogen, R₂ is OH, and R₃ is 1,1-dimethylheptyl;
n is 3, ring A is saturated, R₁ is OH at position C3, R₂ is OH, R₃ is 1,1-dimethylheptyl and R₄ is NO₂ either at ortho or para position to R₂.

Examples of the compound of formula (I) include but are not limited to:
a) 6-(1,1-dimethylpentyl)-8a-methyl-2,3,3a,8a-tetrahydro-1H-8-oxa-cyclo-penta[α]inden-4-ol;
b) 6-(1,1-dimethylheptyl)-8a-methyl-2,3,3a,8a-tetrahydro-1H-8-oxa-cyclo-penta[α]inden-4-ol;
c) 6-(1,1-dimethylheptyl)-2,3,3a,8a-tetrahydro-1H-8-oxa-cyclopenta[α]inden-4-ol;
d) 6-(1,1-dimethylheptyl)-1,8a-dimethyl-2,3,3a,8a-tetrahydro-1H-8-oxa-cyclo-penta[α]inden-4-ol;
e) but-2-enedioic acid mono-[6-(1,1-dimethyl-heptyl)-1,8a-dimethyl-2,3,3a,8a-tetrahydro-1H-8-oxa-cyclopenta[α]inden-4-yl]ester;
f) 3-(1,1-dimethylheptyl)-5a,6,7,8,9,9a-hexahydro-dibenzofuran-1-ol;
g) 3-(1,1-dimethylheptyl)-6-iodo-5a,6,7,8,9,9a-hexahydro-dibenzofuran-1-ol;
h) 3-(1,1-dimethylheptyl)-5a,8,9,9a-tetrahydro-dibenzofuran-1-ol;
i) 3-(1,1-dimethylpentyl)-5a,6,7,8,9,9a-hexahydro-dibenzofuran-1-ol;
j) 3-(1,1-dimethylpentyl)-6-iodo-5a,6,7,8,9,9a-hexahydro-dibenzofuran-1-ol;
k) 3-(1,1-dimethylpentyl)-5a,8,9,9a-tetrahydro-dibenzofuran-1-ol;
l) 3-(1,1-dimethylheptyl)-5a,6,7,8,9,9a-hexahydro-dibenzofuran-1,6-diol;
m) 3-(1,1-dimethylpentyl)-7,7-dimethyl-5a,6,7,8,9,9a-hexahydro-dibenzofuran-1-ol;
n) 3-(1,1-dimethylpentyl)-7,7-dimethyl-5a,6,7,8,9,9a-hexahydro-dibenzofuran-1,6-diol;
o) 3-(1,1-dimethylheptyl)-7,7-dimethyl-5a,6,7,8,9,9a-hexahydro-dibenzofuran-1,6-diol;
p) [3-(1,1-dimethylpentyl)-5a,6,7,8,9,9a-hexahydro-dibenzofuran-1-yloxy]-acetic acid;
q) 3-[3-(1,1-dimethylpentyl)-5a,6,7,8,9,9a-hexahydro-dibenzofuran-1-yloxy]-propane-1,2-diol;
r) 3-(1,1-dimethylpentyl)-5a,6,7,8,9,9a-hexahydro-dibenzofuran-1,6-diol;
s) 3-(2-methyl-[1,3]dithiolan-2-yl)-5a,6,7,8,9,9a-hexahydro-dibenzofuran-1-ol;
t) 4-{2-[3-(1,1-dimethylpentyl)-5a,6,7,8,9,9a-hexahydro-dibenzofuran-1-yloxy]-ethyl}-morpholine;
u) but-2-enedioic acid mono-[3-(1,1-dimethyl-pentyl)-5a,6,7,8,9,9a-hexahydro-dibenzofuran-1-yl]ester;
v) acetic acid 3-(1,1-dimethylpentyl)-5a,6,7,8,9,9a-hexahydro-dibenzofuran-1-yl ester;
w) diethyl phosphoric acid mono-[3-(1,1-dimethyl-pentyl)-5a,6,7,8,9,9a-hexahydro-dibenzo-furan-1-yl]ester;
x) phosphoric acid mono-[3-(1,1-dimethylpentyl)-5a,6,7,8,9,9a-hexahydro-dibenzofuran-1-yl]ester;
y) 3-(1,1-dimethylheptyl)-7,7-dimethyl-5a,6,7,8,9,9a-hexahydro-dibenzofuran-1-ol;
z) [3-(1,1-dimethylheptyl)-5a,6,7,8,9,9a-hexahydro-dibenzofuran-1-yloxy]-acetic acid;
aa) 3-(1,1-dimethylheptyl)-8-isopropylidene-5a-methyl-5a,6,7,8,9,9a-hexahydro-dibenzofuran-1-ol;
ab) 1-(1-hydroxy-5a,6,7,8,9,9a-hexahydro-dibenzofuran-3-yl)-ethanone;
ac) 1-hydroxy-5a,6,7,8,9,9a-hexahydro-dibenzofuran-3-carboxylic acid methyl ester;
ad) 5-[3-(1,1-dimethylpentyl)-5a,6,7,8,9,9a-hexahydro-dibenzofuran-1-yloxymethyl]-1H-tetrazole;
ae) piperidine-3-carboxylic acid 3-(1,1-dimethylpentyl)-5a,6,7,8,9,9a-hexahydro-dibenzofuran-1-yl ester;
af) 4-bromobutyric acid 3-(1,1-dimethylpentyl)-5a,6,7,8,9,9a-hexahydro-dibenzofuran-1-yl ester;
ag) 4-nitrooxy-butyric acid 3-(1,1-dimethylpentyl)-5a,6,7,8,9,9a-hexahydro-dibenzofuran-1-yl ester;
ad) 7-(1,1-dimethylheptyl)-9-hydroxy-3,3-dimethyl-2,3,4a,9b-tetrahydro-1H-dibenzofuran-4-one;
ae) 7-(1,1-dimethylheptyl)-9-hydroxy-2,3,4a,9b-tetrahydro-1H-dibenzofuran-4-one;
af) 7-(1,1-dimethylheptyl)-9-hydroxy-2,3,4a,9b-tetrahydro-1H-dibenzofuran-4-one oxime;
ag) 7-(1,1-dimethylpentyl)-9-methylsulfanylmethoxy-2,3,4a,9b-tetrahydro-1H-dibenzofuran-4-one;
ah) 7-(1,1-dimethylpentyl)-9-hydroxy-2,3,4a,9b-tetrahydro-1H-dibenzofuran-4-one;
ai) 7-(1,1-dimethylpentyl)-9-methylsulfanylmethoxy-2,3,4a,9b-tetrahydro-1H-dibenzofuran-4-one oxime;
aj) 7-(1,1-dimethylpentyl)-9-hydroxy-2,3,4a,9b-tetrahydro-1H-dibenzofuran-4-one oxime;
ak) 7-(1,1-dimethylpentyl)-9-hydroxy-3,3-dimethyl-2,3,4a,9b-tetrahydro-1H-dibenzofuran-4-one;
al) 7-(1,1-dimethylpentyl)-3,3-dimethyl-9-methylsulfanylmethoxy-2,3,4a,9b-tetrahydro-1H-dibenzofuran-4-one;
am) 7-(1,1-dimethylpentyl)-9-hydroxy-3,3-dimethyl-2,3,4a,9b-tetrahydro-1H-dibenzofuran-4-one oxime;
an) 7-(1,1-dimethylpentyl)-3,3-dimethyl-9-methylsulfanylmethoxy-2,3,4a,9b-tetrahydro-1H-dibenzofuran-4-one oxime;
ao) [3-(1,1-dimethylheptyl)-7,7-dimethyl-5a,6,7,8,9,9a-hexahydro-dibenzofuran-1-yloxy]-acetic acid;
ap) but-2-enedioic acid mono-[3-(1,1-dimethylheptyl)-7,7-dimethyl-5a,6,7,8,9,9a-hexahydro-dibenzofuran-1-yl]ester;
aq) 7,7-dimethyl-3-(2-phenethyl-[1,3]dithiolan-2-yl)-5a,6,7,8,9,9a-hexahydro-dibenzofuran-1-ol;
ar) 3-methylamino-propionic acid 3-(1,1-dimethyl-heptyl)-7,7-dimethyl-5a,6,7,8,9,9a-hexahydro-dibenzofuran-1-yl ester;
as) but-2-enedioic acid mono-[7,7-dimethyl-3-(2-phenethyl-[1,3]dithiolan-2-yl)-5a,6,7,8,9,9a-hexahydro-dibenzofuran-1-yl]ester;
at) 3-(1,1-dimethylpentyl)-2,4-dinitro-5a,6,7,8,9,9a-hexahydro-dibenzofuran-1-ol;
au) 3-(1,1-dimethylpentyl)-2-nitro-5a,6,7,8,9,9a-hexahydro-dibenzofuran-1-ol;
av) 3-(1,1-dimethylpentyl)-4-nitro-5a,6,7,8,9,9a-hexahydro-dibenzofuran-1-ol,
aw) 2-(1,1-dimethylheptyl)-5,6,7,8,9,9a-hexahydro-4bH-10-oxa-benzo[α]azulen-4-ol;
ax) 2-(1,1-dimethylheptyl)-5,6,7,8,9,9a-hexahydro-4bH-10-oxa-benzo[α]azulen-4,9-diol;
ay) 2-(1,1-dimethylpentyl)-5,6,7,8,9,9a-hexahydro-4bH-10-oxa-benzo[α]azulen-4-ol;
az) 2-(1,1-dimethylpentyl)-5,6,7,8,9,9a-hexahydro-4bH-10-oxa-benzo[α]azulen-4,9-diol;
ba) 2-(1,1-dimethylpentyl)-4-hydroxy-4-b,5,6,7,8,9a-hexahydro-10-oxa-benzo[α]acazulen-9-one;
bb) 2-(1,1-dimethylheptyl)-4-hydroxy-4-b,5,6,7,8,9a-hexahydro-10-oxa-benzo[α]azulen-9-one;
bc) 4-{2-[2-(1,1-dimethylheptyl)-5,6,7,8,9,9a-hexahydro-4bH-10-oxa-benzo[α]azulen-4-yloxy]-ethyl}-morpholine;
bd) [2-(1,1-dimethylheptyl)-5,6,7,8,9,9a-hexahydro-4bH-10-oxa-benzo[α]azulen-4-yloxy]-acetic acid;
be) but-2-enedioic acid mono-[2-(1,1-dimethylheptyl)-5,6,7,8,9,9a-hexahydro-4bH-10-oxa-benzo[α]azulen-4-yl]ester;
bf) [2-(1,1-dimethylheptyl)-9-hydroxy-5,6,7,8,9,9a-hexahydro-4bH-10-oxa-benzo[α]azulen-4-yloxy]-acetic acid;
bk) 2-(1,1-dimethylheptyl)-5,6,7,9a-tetrahydro-4bH-10-oxa-benzo[α]azulen-4-ol;
bl) 2-(1,1-dimethylheptyl)-5,6,7,8,9,9a-hexahydro-4bH-10-oxa-benzo[α]azulen-4,8,9-triol;
bm) but-2-enedioic acid mono-[9-(3-carboxy-acryloyloxy)-2-(1,1-dimethyl-heptyl)-5,6,7,8,9,9a-hexahydro-4bH-10-oxa-benzo[α]azulen-4-yl]ester;
bn) phosphoric acid mono-[2-(1,1-dimethyl-heptyl)-9-hydroxy-5,6,7,8,9,9a-hexahydro-4bH-10-oxa-benzo[α]azulen-4-yl]ester;
bo) 2-pentyl-5,6,7,8,9,9a-hexahydro-4bH-10-oxa-benzo[α]azulen-4-ol;
bp) 2-pentyl-5,6,7,8,9,9a-hexahydro-4bH-10-oxa-benzo[α]azulen-4,9-diol;
bq) 4-[2-(1,1-dimethylheptyl)-9-hydroxy-5,6,7,8,9,9a-hexahydro-4bH-10-oxa-benzo[α]azulen-4-yloxy]-butyric acid;
br) 2-[2-(1,1-dimethylheptyl)-9-hydroxy-5,6,7,8,9,9a-hexahydro-4bH-10-oxa-benzo[α]azulen-4-yloxy]-N,N-diethyl-acetamide;
bs) 2-(1,1-dimethylheptyl)-4-(2-morpholin-4-yl-ethoxy)-5,6,7,8,9,9a-hexahydro-4bH-10-oxa-benzo[α]azulen-9-ol;
bt) 2-(1,1-dimethylheptyl)-4-(2H-tetrazol-5-ylmethoxy)-5,6,7,8,9,9a-hexahydro-4bH-10-oxa-benzo[α]azulen-9-ol;
bu) 2-(1,1-dimethylheptyl)-4-hydroxy-5,6,7,8,9,9a-hexahydro-4bH-10-oxa-benzo[α]azulen-5-carboxylic acid methyl ester;
bv) 2-(1,1-dimethylheptyl)-4,9-dihydroxy-5,6,7,8,9,9a-hexahydro-4bH-10-oxa-benzo[α]azulen-5-carboxylic acid;
bw) 2-(1,1-dimethylheptyl)-5-hydroxymethyl-5,6,7,8,9,9a-hexahydro-4bH-10-oxa-benzo [α]azulen-4-ol;
bx) [2-(1,1-dimethylheptyl)-4-hydroxy-5,6,7,8,9,9a-hexahydro-4bH-10-oxa-benzo[α]azulen-5-yl]-acetic acid methyl ester;
by) 2-(1,1-dimethylheptyl)-4-hydroxy-4b,5,6,7,8,9a-hexahydro-10-oxa-benzo[α]azulen-9-one oxime;
bz) 2-(1,1-dimethylheptyl)-9-iodo-5,6,7,8,9,9a-hexahydro-4bH-10-oxa-benzo[α]azulen-4-ol;
ca) [2-(2-methoxy-ethoxy)-ethoxy]-acetic acid 2-(1,1-dimethyl-heptyl)-9-hydroxy-5,6,7,8,9,9a-hexahydro-4bH-10-oxa-benzo[α]azulen-4-yl ester;
cb) but-2-enedioic acid mono-[2-(1,1-dimethylheptyl)-9-iodo-5,6,7,8,9,9a-hexahydro-4bH-10-oxa-benzo[α]azulen-4-yl]ester;
cc) 2-(1,1-dimethylheptyl)-4-hydroxy-5,6,7,8,9,9a-hexahydro-4bH-10-oxa-benzo[α]azulen-5-carboxylic acid;
cd) 9-amino-2-(1,1-dimethylheptyl)-5,6,7,8,9,9a-hexahydro-4bH-10-oxa-benzo[α]azulen-4-ol;
ce) 9-Amino-2-(1,1-dimethyl-heptyl)-5,6,7,8,9,9a-hexahydro-4bH-10-desoxy-benzo[α]azulen-4-ol;
cf) 2-(1,1-dimethylheptyl)-3-nitro-5,6,7,8,9,9a-hexahydro-4bH-10-oxa-benzo[α]azulen-4,9-diol;
cg) 2-(1,1-dimethylheptyl)-1-nitro-5,6,7,8,9,9a-hexahydro-4bH-10-oxa-benzo[α]azulen-4,9-diol;
ch) 3-(1,1-dimethylheptyl)-6,7,8,9-tetrahydro-dibenzofuran-1-ol;
ci) but-2-enedioic acid mono-[3-(1,1-dimethylheptyl)-6,7,8,9-tetrahydro-dibenzofuran-1-yl]ester;
cj) 3-(1,1-dimethylpentyl)-6,7,8,9-tetrahydro-dibenzofuran-1-ol;
ck) 7-(1,1-dimethylheptyl)-9-methoxy-1,1-dimethyl-2,3-dihydro-1H-dibenzofuran-4-one;
cl) 7-(1,1-dimethylheptyl)-9-methoxy-2,2-dimethyl-2,3-dihydro-1H-dibenzofuran-4-one;
cm) but-2-enedioic acid mono-[3-(1,1-dimethylpentyl)-6,7,8,9-tetrahydro-dibenzofuran-1-yl]ester;
cn) 7-(1,1-dimethylheptyl)-9-hydroxy-1,1-dimethyl-2,3-dihydro-1H-dibenzofuran-4-one; and
co) 3-(1,1-dimethylheptyl)-7,7-dimethyl-6,7,8,9-tetrahydro-dibenzofuran-1-ol.

According to another aspect, the present invention provides a compound of formula (II):

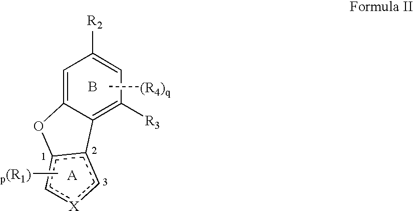

and stereoisomers, pharmaceutically acceptable salts, esters, polymorphs or solvates of said compounds; wherein [symbol] represents a single or double bond and wherein X, R₁ through R₄ and m, n, p and q are as defined for formula (I) with the provisos that (a) A is not a phenyl ring; and (b) when n is 2, and R₁ at C2 is isopropyl then R₁ at C5 is other than methyl.

According to certain embodiments, the present invention provides a compound of formula (II) wherein n is an integer from 1 to 3, ring A is unsaturated, R₁ is selected from the group consisting of hydrogen, carbonyl, and R, R₂ is OR, and R₃ is a saturated or unsaturated, linear, branched or cyclic C₁-C₁₂ alkyl wherein R is as previously defined.

According to additional embodiments, the present invention provides a compound of formula (II) wherein n is 2, ring A is unsaturated and the double bond is positioned between C1 and C2, R₁ is selected from the group consisting of hydrogen, carbonyl and CH₃, R₂ is OCH₃ and R₃ is 1,1-dimethylheptyl.

According to exemplary embodiments, the present invention provides a compound of formula (II) wherein n is 2, ring A is unsaturated and the double bond is positioned between C1 and C2, R₁ is selected from the group consisting of a carbonyl at position C6 and gem-dimethyl at position C3 or C4, R₂ is OCH₃ and R₃ is 1,1-dimethylheptyl.

Examples of the compound of formula (II) include but are not limited to:

a) 9-(1,1-dimethylheptyl)-7-methoxy-1,1-dimethyl-2,3-dihydro-1H-dibenzofuran-4-one; and b) 9-(1,1-dimethylheptyl)-7-methoxy-2,2-dimethyl-2,3-dihydro-1H-dibenzofuran-4-one.

The compounds of the invention can be used for the preparation of a medicament either as the active ingredient, as is, or in the form of their pharmaceutically acceptable salts, esters, solvates and derivatives.

According to a further aspect, the present invention provides a pharmaceutical composition comprising as an active ingredient an effective amount of a compound of formula (I):

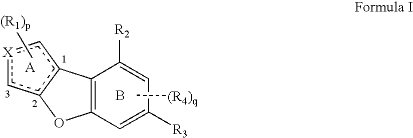

and stereoisomers, pharmaceutically acceptable salts, esters, polymorphs or solvates of said compounds; wherein [symbol] represents a single or double bond and wherein X, R₁ through R₄ and m, n, p and q are as defined for formula (I) with the provisos that (a) A is not a phenyl ring; (b) when n is 1 and R₁ is a phenyl at position C2, then the optional R₁ at position C1 is other than hydroxyl; and (c) when n is 2, R₁ is methyl and hydroxyl at C3 and isopropenyl at C6, then R₂ is other than OH, OCH₃ and OC(O)CH₃.

According to certain embodiments, the present invention provides a pharmaceutical composition comprising as an active ingredient an effective amount of a compound of formula (I) as defined therein, wherein the exemplary substituents X and R₁ through R₄ are as defined for formula (I).

According to exemplary embodiments, the present invention provides a pharmaceutical composition comprising as an active ingredient an effective amount of a compound of formula (I) selected from the group consisting of compounds a) to co) as defined above.

According to another aspect, the present invention provides a pharmaceutical composition comprising as an active ingredient an effective amount of a compound of formula (II):

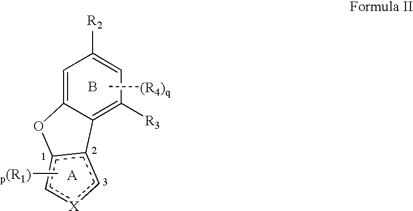

and stereoisomers, pharmaceutically acceptable salts, esters, polymorphs or solvates of said compounds; wherein [symbol] represents a single or double bond and wherein X, R₁ through R₄ and m, n, p and q are as defined for formula (II) with the proviso that A is not a phenyl ring.

According to certain embodiments, the present invention provides a pharmaceutical composition comprising as an active ingredient an effective amount of a compound of formula (II) as defined therein, wherein the exemplary substituents X and R₁ through R₄ are as defined for formula (II).

According to exemplary embodiments, the present invention provides a pharmaceutical composition comprising as an active ingredient an effective amount of a compound of formula (II) selected from the group consisting of 9-(1,1-dimethylheptyl)-7-methoxy-1,1-dimethyl-2,3-dihydro-1H-dibenzofuran-4-one and 9-(1,1-dimethylheptyl)-7-methoxy-2,2-dimethyl-2,3-dihydro-1H-dibenzofuran-4-one.

Pharmaceutical compositions of the present invention can include in addition to the above-defined compounds of formulae (I) and (II), thickeners, carriers, buffers, diluents, surface active agents, preservatives and the like, all as well known in the art, necessary to produce a physiologically acceptable and stable formulation.

In addition, the present invention provides a method of treatment which comprises administering to a subject in need thereof a prophylactically and/or therapeutically effective amount of aforesaid compounds or pharmaceutical compositions comprising them.

According to a further aspect, the present invention provides a method of preventing, alleviating or treating medical conditions as above described, which comprises administering to a subject in need thereof a prophylactically and/or therapeutically effective amount of a compound of formula (I) or (II) as above defined, or a pharmaceutical composition comprising said compound as an active ingredient.

The principles of the present invention will be more fully understood by reference to the following examples, which illustrate preferred embodiments of the invention and are to be construed in a non-limitative manner.

### EXAMPLES

The following examples are provided in order to demonstrate and further illustrate certain preferred embodiments and aspects of the present invention and are not to be construed as limiting the scope thereof.

For convenience and better understanding, the section of the Examples is divided into two subsections: the Chemical Section describing the synthesis of compounds of the invention, some of their properties and their formulation; and the Biological Section describing the biological activity of the compounds.

In the experimental disclosure which follows, the following abbreviations apply: N (normal); M (molar); mM (millimolar); μM (micromolar); mmol (millimole); kg (kilograms); g (grams); mg (milligrams); μg (micrograms); ng (nanograms); pg (picograms); ml (milliliters); μl (microliters); mm (millimeters); μm (micrometers); hr/s (hour/s); min (minute/s); MHz (mega Hertz); IR (infra red); NMR (nuclear magnetic resonance); MS (mass spectroscopy); HPLC (high pressure liquid chromatography); TLC (thin layer chromatography); ACN (acetonitrile); Cs₂CO₃ (cesium carbonate); DCC (dicyclohexylcarbodiimide); DCM (dichloromethane); DMAP (N,N-dimethyl-amino-pyridine); DMF (dimethyl formamide); EA (ethyl acetate); Et₂O (ethyl ether); IPA (isopropanol); PE (petroleum ether); TEA (triethylamine); THF (tetrahydrofuran); p-TsOH (para-toluene sulfonic acid); anh. (anhydrous); eq. (equivalent); sat. (saturated); ppm (part per million); ° C. (degrees Centigrade); RH (relative humidity); RT (room temperature); i.m. (intramuscularily); i.p. (intraperitoneally); i.v. (intravenously); p.o. (per os); s.c. (subcutaneously); AUC (area under the curve); SD (standard deviation); SEM (standard error of the mean); NA (not available or not tested); NB (no binding).

### Chemical Section

In the synthetic examples, unless otherwise noted, the reaction was worked-up as follows. Upon completion of the reaction, as monitored by TLC (20% EA in PE), the mixture was washed twice with a solution of saturated sodium bicarbonate and then once with brine. The organic phase was separated, dried and evaporated, and the crude product was isolated and purified by column chromatography on silica gel with 20% ethyl acetate in petroleum ether as the eluent. The level of purity was further confirmed using HPLC. All compounds were characterized by mass spectroscopy (MS) and resonances were assigned by 300 or 600 MHz nuclear magnetic resonance (NMR), as appropriate. MS and NMR spectra were consistent with the assigned structure.

In the following examples, various 5-substituted resorcinols were used for the preparation of the novel compounds of the invention. Though the following examples disclose specific resorcinolic reagents, it is clear that a diversity of resorcinol moieties could be used in the same or in alternative synthetic procedures known to persons skilled in the art of medicinal chemistry. Methods for the synthesis of such resorcinol derivatives are known and were previously disclosed. For instance the synthesis of 5-(1′,1′-dimethylheptyl)-resorcinol is detailed in international patent application WO 2004/050011, incorporated by reference herein in its entirety, whereas the preparation of additional resorcinol derivatives is described in international patent application WO 03/063758, incorporated by reference herein in its entirety. Alternative synthetic methods exist for the preparation of said compounds.

In the present specification and claims which follow, compounds of the invention may be referred to by a combination of capital letters and numbers rather than by their fall chemical names, which were determined using ChemDraw Ultra® 7.0.1 (CambridgeSoft Corporation). The prefixes C5S, C6S, and C7S, corresponding to n is 0, 1 and 2 in the following schemes and to n is 1, 2 and 3 in formulae (I) and (II), indicate that the A ring fused to the benzofuran moiety is saturated between C1 and C2 and is either cyclopentyl (C5), cyclohexyl (C6) or cycloheptyl (C7). Addition of the letter N to said prefixes indicate that the B ring of the benzofuran moiety is further substituted beyond R₂ and R₃ as specified in formulae (I) and (II). Compounds prefixed C6M comprise a 6 membered A ring with a double bond between C1 and C2.

### Example 1

### Method A

### Coupling and Cyclization

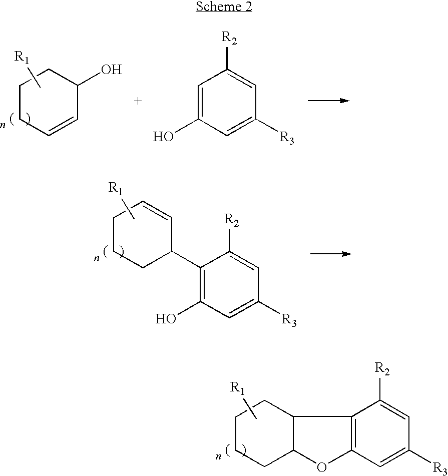

### Synthesis of Compound C6S-1: 3-(1,1-Dimethylheptyl)-5a,6,7,8,9,9a-hexahydro-dibenzofuran-1-ol

The synthesis of compound C6S-1 is as depicted in Scheme 2 when n is 1, R₁ is hydrogen, R₂ is hydroxyl, and R₃ is 1,1-dimethylheptyl.

A mixture of 5-(1,1-dimethylheptyl)-resorcinol (1,174.2 mg, 4.97 mmol), 2-cyclohexen-1-ol (690 mg, 7.03 mmol) and methanesulfonic acid (110 mg, 0.79 mmol) in 100 ml of dichloromethane (DCM) was stirred for 4 hrs at RT. The reaction progress was monitored by TLC. Upon completion of the reaction, the mixture was washed twice with a solution of saturated sodium bicarbonate and then once with brine. After phase separation and evaporation of the organic phase, the crude product was isolated and purified by column chromatography on silica gel with 20% EA in PE as the eluent. The purified 2-(2-cyclohexenyl)-5-(1,1-dimethylheptyl)-resorcinol was obtained at a yield of 81%.

A mixture comprising the previously obtained 2-(2-cyclohexenyl)-5-(1,1-dimethyl-heptyl)-resorcinol and 0.1 ml of boron trifluoride etherate in 50 ml of dry DCM was stirred for about 12 hrs at RT. The reaction was worked-up as described above. Compound C6S-1 was afforded at a yield of 83%.

Using this method the cycloalkenols cyclopent-2-enol, cyclohex-2-enol, cyclohept-2-enol, 4,4-dimethyl-cyclohex-2-enol, 2-hydroxy-cyclohept-3-enecarboxylic acid methyl ester, (2-hydroxy-cyclohept-3-enyl)-acetic acid methyl ester, 2-methyl-2-cyclopenten-1-ol, 2,3-dimethyl-cyclopent-2-en-1-ol and (−)-carveol could be coupled with any of the following resorcinols: 3-(1′,1′-dimethylheptyl)-benzene-1,5-diol, 3-(1′,1′-pentyl)-benzene-1,5-diol, 3-(1′,1′-dimethylpentyl)-benzene-1,5-diol, 5-(2-methyl-[1,3]dithiolan-2-yl)-benzene-1,3-diol, 5-(2-phenethyl-[1,3]dithiolan-2-yl)-benzene-1,3-diol and 3,5-dihydroxy-benzoic acid methyl ester. The cycloalkenols used in this procedure were obtained by reduction with LiAlH₄ of the corresponding α,β unsaturated ketones according to methods known in the art. The compounds listed below were therefore prepared in a similar fashion.
C5S-1 6-(1,1-dimethylpentyl)-8a-methyl-2,3,3a,8a-tetrahydro-1H-8-oxa-cyclopenta[α]inden-4-ol
C5S-3 6-(1,1-dimethylheptyl)-2,3,3a,8a-tetrahydro-1H-8-oxa-cyclopenta[α]inden-4-ol
C6S-3 3-(1,1-dimethylpentyl)-5a,6,7,8,9,9a-hexahydro-dibenzofuran-1-ol
C6S-6 3-(1,1-dimethylpentyl)-7,7-dimethyl-5a,6,7,8,9,9a-hexahydro-dibenzofuran-1-ol
C6S-12 3-(1,1-dimethylpentyl)-9a-methyl-5a,6,7,8,9,9a-hexahydro-dibenzofuran-1-ol
C6S-17 3-(1,1-dimethylheptyl)-7,7-dimethyl-5a,6,7,8,9,9a-hexahydro-dibenzofuran-1-ol
C6S-21 1-hydroxy-5a,6,7,8,9,9a-hexahydro-dibenzofuran-3-carboxylic acid methyl ester
C6S-38 7,7-dimethyl-3-(2-phenethyl-[1,3]dithiolan-2-yl)-5a,6,7,8,9,9a-hexahydro-dibenzofuran-1-ol
C7S-1 2-(1,1-dimethylheptyl)-5,6,7,8,9,9a-hexahydro-4bH-10-oxa-benzo[α]azulen-4-ol
C7S-3 2-(1,1-dimethylpentyl)-5,6,7,8,9,9a-hexahydro-4bH-10-oxa-benzo[α]azulen-4-ol
C7S-14 2-pentyl-5,6,7,8,9,9a-hexahydro-4bH-10-oxa-benzo[α]azulen-4-ol
C7S-20 2-(1,1-dimethylheptyl)-4-hydroxy-5,6,7,8,9,9a-hexahydro-4bH-10-oxa-benzo[α]azulen-5-carboxylic acid methyl ester
C7S-23/4 [2-(1,1-dimethylheptyl)-4-hydroxy-5,6,7,8,9,9a-hexahydro-4bH-10-oxa-benzo[α]azulen-5-yl]-acetic acid methyl ester

According to a similar method wherein there was no intermediate step as shown in Scheme 2, the following compounds were directly obtained.
C5S-2 6-(1,1-dimethylheptyl)-8a-methyl-2,3,3a,8a-tetrahydro-1H-8-oxa-cyclo-penta[α]inden-4-ol
C5S-4 6-(1,1-dimethylheptyl)-1,8a-dimethyl-2,3,3a,8a-tetrahydro-1H-8-oxa-cyclo-penta[α]inden-4-ol
C6S-19 3-(1,1-dimethylheptyl)-8-isopropylidene-5a-methyl-5a,6,7,8,9,9a-hexahydro-dibenzofuran-1-ol

### Example 2

### Method B

### Alkylation and Cyclization

### a) First Procedure

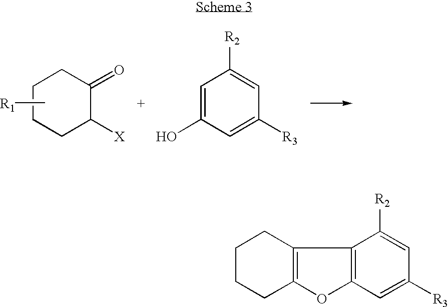

### Synthesis of Compound C6M-1: 3-(1,1-Dimethylheptyl)-6,7,8,9-tetrahydro-dibenzo-furan-1-ol

Compound C6M-1 was prepared as depicted in Scheme 3 when R₁ is hydrogen, X is chlorine, R₂ is hydroxyl and R₃ is 1,1-dimethylheptyl.

Into a 500 ml round bottom flask 2-chlorocyclohexanone (3.5 g, 26 mmol), 5-(1,1-dimethylheptyl)-resorcinol (6.2 g, 26 mmol), anhydrous potassium carbonate (3.5 g, 25 mmol) in 150 ml of dry acetone were added. The reaction mixture was refluxed for 10 hrs. The reaction progress was monitored by TLC (10% EA in PE). Upon completion of the reaction, the mixture was evaporated to dryness and 100 ml of ethyl acetate were added followed by 50 ml of 10% HCl. After phase separation and removal of the organic solvent, the crude product was isolated and purified in two steps, first by column chromatography (10% EA in PE) and then by biotage chromatography. Purification afforded 320 mg of pure compound C6M-1.

Using this method with a different resorcinol, the following compound was prepared and, after lyophilization, 143 mg of compound C6M-3 were obtained as a yellow powder. C6M-3 3-(1,1-dimethylpentyl)-6,7,8,9-tetrahydro-dibenzo-furan-1-ol

### b) Second Procedure

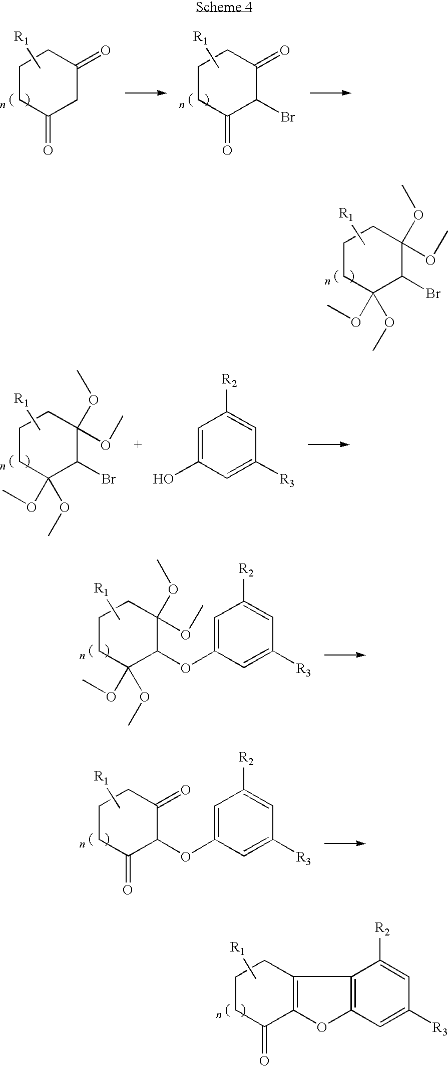

### Synthesis of Compounds C6M-4 and C6M-5: 7-(1,1-Dimethylheptyl)-9-methoxy-1,1-dimethyl-2,3-dihydro-1H-dibenzofuran-4-one and 9-(1,1-Dimethylheptyl)-7-methoxy-1,1-dimethyl-2,3-dihydro-1H-dibenzofuran-4-one, respectively

Compounds C6M-4 and C6M-5 were prepared as depicted in Scheme 4 wherein the cyclohexadione ring is substituted with gem-dimethyl, R₂ is methoxy and R₃ is 1,1-dimethylheptyl.

2-bromo-4,4-dimethyl-1,3-cyclohexadione (1). To a cooled stirred suspension of 4,4-dimethyl-1,3-cyclohexadione (2 g, 14.3 mmol) in 75 ml of diethyl ether at −10° C. was added bromine (732 μL, 14.3 mmol) slowly by syringe. After complete addition, the solution was stirred for an additional 30 minutes, whereupon it was quenched by the addition of water. The layers were separated, and the organic layer was washed repeatedly with water, and a ½ saturated solution of sodium bicarbonate. The aqueous layers were combined and further extracted with ether several times. The combined organic fractions were dried over sodium sulfate (Na₂SO₄ anh.). Filtration followed by removal of the solvent under reduced pressure afforded 2.5 grams of a light yellow solid (1), which was used in the next step without further purification.

3-bromo-2,2,4,4-tetramethoxy-1,1-dimethyl cyclohexane (2). To a stirred solution of compound I (1.5 g, 6.8 mmol) in absolute methanol were added trimethyl orthoformate (5 ml, 64 mmol) and a catalytic amount of p-TsOH. The reaction was heated to reflux and allowed to stir overnight under an atmosphere of nitrogen. The following day, the reaction was cooled to room temperature and the consumption of all the starting material was confirmed by TLC (eluent: EA). The solvent was removed under reduced pressure and the oily yellow residue was redissolved into ethyl acetate. The organic solution was repeatedly washed with water, ½ saturated aqueous sodium bicarbonate, and finally brine. The organic fraction was dried (Na₂SO₄ anh.), decanted and the solvent removed on the roto-evaporator affording 1.6 grams compound (2) as a yellow solid.

1-(2,2,6,6-tetramethoxy-3,3-dimethyl)-cyclohexyl-3′-methoxy-4′-(1″,1″-dimethylheptyl)-phenyl ether (3). To a stirred solution of 1,1-dimethyl-1-(3′-hydroxy-5′-methoxy)-phenyl hexane (500 mg, 2 mmol) in 10 ml acetonitrile (ACN) was added Cs₂CO₃ (1.5 g, 3 mmol). The stirring solution was heated to 75° C. for 0.5 hr under a N₂ atmosphere, at which point 2 (680 mg, 2.2 mmol) was added. After being stirred at 75° C. for an additional hr, 30 ml of DMF were added and the reaction temperature was raised up to 150° C. for an additional 10 hrs. After cooling to room temperature, the majority of the solvent was removed on a roto-evaporator. The oily residue was redissolved in ethyl acetate and washed repeatedly with water and dilute HCl. The organic fraction was dried (Na₂SO₄ anh.), decanted and the solvent removed with a roto-evaporator. The crude material was purified with column chromatography to afford 820 mg of the desired product (3).

2[-3-(1,1-dimethylheptyl)-5-methoxy-phenoxy]-4,4-dimethyl-cyclohexane-1,3-dione (4). A solution of 3 (780 mg, 1.6 mmol) with 3 equivalents of p-TsOH in acetone was stirred at RT over a period of 24 to 48 hrs. The reaction was monitored by TLC for the disappearance of the starting material. After complete consumption of compound 3, the acetone was removed via roto-evaporation and the residue dissolved in ethyl ether. The ether was washed with water and saturated sodium bicarbonate followed by drying over sodium sulfate. Purification of the final product was achieved by column chromatography (eluent: 80:20 PE:EA [symbol] 70:30 PE:EA) affording 400 mg of 4.

C6M-4 and C6M-5. In 4 ml of polyphosphoric acid was stirred compound 4 (235 mg, 0.68 mmol) at 95° C. for 4 hrs under a N₂ atmosphere. The reaction was cooled to room temperature, diluted with water and extracted with diethyl ether. The organic fractions were combined and washed repeatedly with water. The organic layer was dried over sodium sulfate, decanted to remove solids, and the solvent removed under reduced pressure. The crude material contained a roughly 50/50 distribution of the two regioisomers. The two regioisomers were separated with column chromatography (eluent: 95:05 PE:EA [symbol] 90:10 PE:EA [symbol] 80: PE:EA) to afford the linear compound of formula (I) C6M-4 as the first fraction, followed by the angular compound of formula (II) C6M-5 in a 41% total yield.

Using this method with a different cyclohexadione, 2-bromo-5,5-dimethyl-1,3-cyclohexadione, the following compounds were prepared at 40% total yield, where C6M-6 is the linear regioisomer of formula (I) and C6M-7 is the angular regioisomer of formula (II).
C6M-6 7-(1,1-dimethylheptyl)-9-methoxy-2,2-dimethyl-2,3-dihydro-1H-dibenzofuran-4-one
C6M-7 9-(1,1-dimethylheptyl)-7-methoxy-2,2-dimethyl-2,3-dihydro-1H-dibenzofuran-4-one

### c) Third Procedure

### Synthesis of Compound C6M-9: 7-(1,1-Dimethylheptyl)-9-hydroxy-1,1-dimethyl-2,3-dihydro-1H-dibenzofuran-4-one

Compound C6M-9 was prepared by substitution of the methoxyl of C6M-4, prepared as described above, by an hydroxyl.

To a stirred solution of C6M-4 (60 mg, 0.28 mmol) in 5 ml of dry CH₂Cl₂ cooled to 0° C. was added BBr₃ (162 mg, 1.11 mmol) slowly by syringe. The reaction solution was warmed slowly to room temperature and was stirred at RT for an additional period of 16 hrs. The reaction was quenched by the careful addition of distilled water followed by a small amount of saturated sodium bicarbonate solution. The quenched reaction was stirred until its color lightened to yellow, whereupon the organic layer was separated from the aqueous. The aqueous layer was extracted several times with methylene chloride, and the combined methylene chloride fractions were washed with saturated sodium bicarbonate and brine. The organic fraction was dried over sodium sulfate and the solvent removed via roto-evaporator to afford the crude product. Purification with column chromatography (10% EA in PE) afforded 45 mg pure compound C6M-9 as an off white solid.

### Example 3

### Method C

### Oxidation and Cyclization

### a) First Procedure

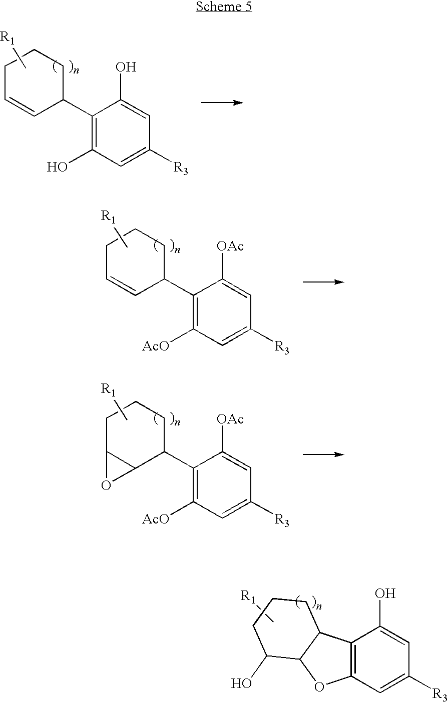

### Synthesis of Compound C6S-5: 3-(1,1-Dimethylheptyl)-5a,6,7,8,9,9a-hexahydro-dibenzofuran-1,6-diol

The synthesis of compound C6S-5 is as depicted in Scheme 5 when n is 1, R₁ is hydrogen and R₃ is 1,1-dimethylheptyl.

To a mixture of 2-(cyclohex-2-enyl)-5-(1,1-dimethylheptyl)-benzene-1,3-diol (945 mg, 2.99 mmol) and ethyldiisopropylamine in 50 ml of DCM (1,012 mg, 7.84 mmol), acetic anhydride (765 mg, 7.5 mmol) was added dropwise and the resulting mixture was stirred for 12 hrs at RT. Upon completion of the reaction, the organic solvent was evaporated under vacuum and the crude product purified by column chromatography. The purified acetic acid 3-acetoxy-2-cyclohex-2-enyl-5-(1,1-dimethylheptyl)-phenyl ester was obtained at a yield of 74%.

To a mixture comprising the previously obtained acetic acid 3-acetoxy-2-cyclohex-2-enyl-5-(1,1-dimethylheptyl)-phenyl ester in 20 ml of chloroform, m-chloroperbenzoic acid, (900 mg 3.66 mmol) in 30 ml of chloroform was added and the resulting mixture was refluxed for about one hr and stirred for 3 hrs at RT. The reaction mixture was washed, and the product was isolated and purified by column chromatography. The purified acetic acid 3-acetoxy-5-(1,1-dimethylheptyl)-2-(7-oxa-bicyclo [4.1.0]hept-2-yl)-phenyl ester was obtained at a yield of 59%.

A mixture comprising the previously obtained acetic acid 3-acetoxy-5-(1,1-dimethyl-heptyl)-2-(7-oxa-bicyclo[4.1.0]hept-2-yl)-phenyl ester) and sodium hydrogen carbonate (250 mg, 2.97 mmol) in 30 ml of methanol and 5 ml of water was refluxed for 3 hrs. The reaction progress was monitored by TLC. The reaction mixture was extracted twice with ethyl acetate and washed once with brine. After separation, the organic layer was dried over sodium sulfate, the solvent was evaporated and the crude oil was purified by column chromatography. Compound C6S-5 was afforded at a yield of 87%.

According to a similar method, using different starting materials, the following compounds were obtained.
C6S7 3-(1,1-dimethylpentyl)-7,7-dimethyl-5a,6,7,8,9,9a-hexahydro-dibenzofuran-1,6-diol
C6S-8 3-(1,1-dimethylheptyl)-7,7-dimethyl-5a,6,7,8,9,9a-hexahydro-dibenzofuran-1,6-diol
C6S-11 3-(1,1-dimethylpentyl)-5a,6,7,8,9,9a-hexahydro-dibenzofuran-1,6-diol
C7S-2 2-(1,1-dimethylheptyl)-5,6,7,8,9,9a-hexahydro-4bH-10-oxa-benzo[α]azulen-4,9-diol
C7S-4 2-(1,1-dimethylpentyl)-5,6,7,8,9,9a-hexahydro-4bH-10-oxa-benzo[α]azulen-4,9-diol
C7S-15 2-pentyl-5,6,7,8,9,9a-hexahydro-4bH-10-oxa-benzo[α]azulen-4,9-diol
C7S-21 2-(1,1-dimethylheptyl)-4,9-dihydroxy-5,6,7,8,9,9a-hexahydro-4bH-10-oxa-benzo[α]azulen-5-carboxylic acid

### b) Second Procedure

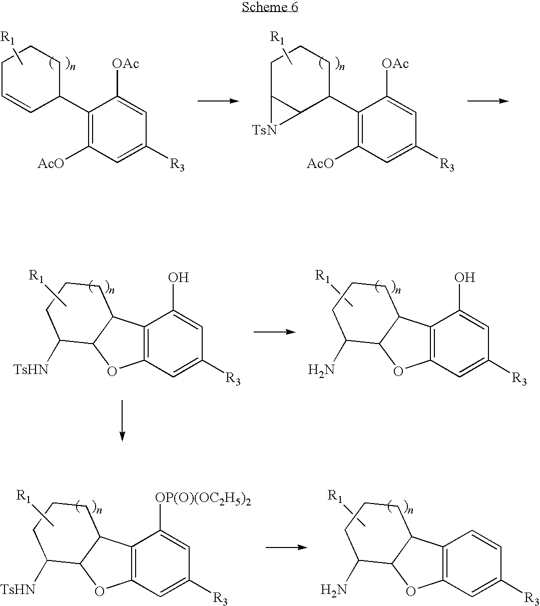

### Synthesis of Compounds C7S-32 and C7S-33: 9-Amino-2-(1,1-dimethylheptyl)-5,6,7,8,9,9a-hexahydro-4bH-10-oxa-benzo[α]azulen-4-ol and 9-amino-2-(1,1-dimethylheptyl)-5,6,7,8,9,9a-hexahydro-4bH-10-desoxy-benzo[α]azulen-4-ol, Respectively.

The synthesis of compounds C7S-32 and C7S-33 are as depicted in Scheme 6 when n is 1, R₁ is hydrogen and R₃ is 1,1-dimethylheptyl and the final synthetic step of C7S-32 is shown in the upper line, whereas the final synthetic steps of C7S-33 are depicted in the lower part of the scheme.

To the mixture 3,5-diacetoxy-2-(cyclohept-2-enyl)-5-(1,1-dimethylheptyl)benzene (1) (1,021 mg, 2.48 mmol) in 50 ml of acetonitrile chloramine-T (1,021 mg, 3.63 mmol) and benzyltriethylammonium tribromide (480 mg, 1.23 mmol) were added and the resulting mixture was stirred for 48 hrs. The reaction progress was monitored by TLC. The white solid was filtrated, organic solvent was evaporated under reduced pressure and the crude product purified by column chromatography (30% EA in PE). Yield of (2) 85%.

To the mixture of (2) in 20 ml of methanol sodium hydroxide (400 mg, 10 mmol) in 3 ml of water was added and the resulting mixture was stirred for 3 hrs at RT. Diethyl ether was added and the reaction mixture was washed twice with 1N HCl solution and then with brine. After phase separation and evaporation of the organic phase, the product was isolated and purified by column chromatography (30% EA in PE). Yield of (3) 69%.

A solution of (3) in 50 ml of THF was cooled with acetone dry ice (−70° C.) ammonium was condensed during 2 hrs. Lithium metal was added dropwise until blue color disappeared. Mixture was stirred at low temperature for 2 hrs and was then heated to RT. Solution of ammonium chloride was added and mixture was extracted with diethyl ether. Organic layer was washed with brine and dried over sodium sulfate, solvent was evaporated and crude oil was purified by column chromatography (30% THF in PE). Compound C7S-32 was obtained at a yield of 69%.

Compound C7S-33 was prepared similarly up to compound (3) which was further reacted as follows. To the solution of potassium t-butoxide (224 mg, 2.0 mmol) in 30 ml of THF, compound (3) (498 mg, 1 mmol) in 20 ml of THF was added dropwise and, after 30 min, diethylchlorophosphate (190 mg, 1.1 mmol). The resulting mixture was stirred overnight. Water and then diethyl ether were added and the reaction mixture was washed twice with 1N HCl solution and then with brine. After phase separation and evaporation of the organic phase, the product was isolated and purified by column chromatography with 30% ethyl acetate in petroleum ether as eluent. Yield of (4) 82%. Compound (4) was further deprotected with Lithium metal in ammonium liquid at −70° C., as previously described. Compound C7S-33 was obtained at a yield of 69%.

### Example 4

### Method D

### Alkylation of Phenolic Hydroxyl

While the methods described in Examples 1 to 3 related to the preparation of benzofuran derivatives, the following procedures generally relate to various chemical modifications that were performed on such compounds.

### a) First Procedure

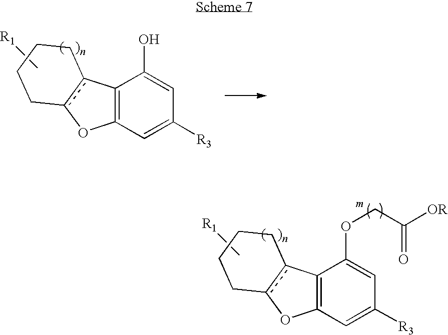

### Synthesis of Compound C6S-9: [3-(1,1-Dimethylpentyl)-5a,6,7,8,9,9a-hexahydro-dibenzofuran-1-yloxy]-acetic acid

The synthesis of compound C6S-9 is based on the eterification of compound C6S-3, which was prepared as described in Example 1. C6S-9 was prepared as generally depicted in Scheme 7 when n is 1, there is a single bond between C1 and C2, m is 1, R₁ and R are hydrogen atoms and R₃ is 1,1-dimethylpentyl.

Compound C6S-3 (0.04 g, 0.138 mmol) was dissolved in 10 ml ACN containing solid Cs₂CO₃ (0.1 g, 0.31 mmol), and refluxed while stirring for 2 hrs. Ethyl bromoacetate (0.25 ml, 1.49 mmol) was then added dropwise to the reaction mixture, which was then stirred at reflux for 3 hrs under N₂ atmosphere. Ethyl acetate (30 ml) was added to the mixture, which was washed twice with brine and once with water. The organic layer was dried over Na₂SO₄ (anh.), filtered and evaporated under reduced pressure to afford 97 mg of crude [3-(1,1-dimethylpentyl)-5a,6,7,8,9,9a-hexahydro-dibenzofuran-1-yloxy]-acetic acid ethyl ester.

The crude material obtained in previous step was dissolved in 15 ml of methanol. Water (5 ml) and K₂CO₃ (0.5 g) were then added. The reaction mixture was stirred for 24 hrs at RT. HCl (1 N) was added until cloudiness. Following extraction with EA (×2), the combined organic layers were washed with water (×2). The solvent was evaporated after drying over Na₂SO₄, yielding 41 mg of pure C6S-9.

According to a similar method, using different starting materials, the following compounds were obtained.
C6S-18 [3-(1,1-dimethylheptyl)-5a,6,7,8,9,9a-hexahydro-dibenzofuran-1-yloxy]-acetic acid
C6S-36 [3-(1,1-dimethylheptyl)-7,7-dimethyl-5a,6,7,8,9,9a-hexahydro-dibenzofuran-1-yloxy]-acetic acid
C7S-8 [2-(1,1-dimethylheptyl)-5,6,7,8,9,9a-hexahydro-4bH-10-oxa-benzo[α]azulen-4-yloxy]-acetic acid
C7S-10 [2-(1,1-dimethylheptyl)-9-hydroxy-5,6,7,8,9,9a-hexahydro-4bH-10-oxa-benzo[α]azulen-4-yloxy]-acetic acid
C7S-16 4-[2-(1,1-dimethylheptyl)-9-hydroxy-5,6,7,8,9,9a-hexahydro-4bH-10-oxa-benzo[α]azulen-4-yloxy]-butyric acid

### b) Second Procedure

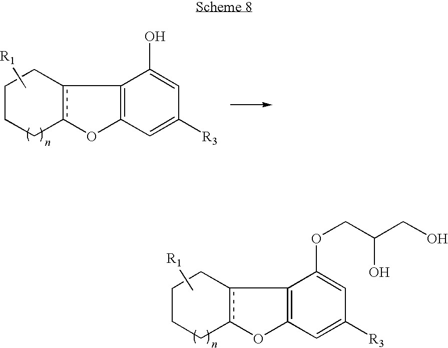

### Synthesis of Compound C6S-10: 3-[3-(1,1-Dimethylpentyl)-5a,6,7,8,9,9a-hexahydro-dibenzofuran-1-yloxy]-propane-1,2-diol

The synthesis of compound C6S-10 is based on the eterification of compound C6S-3, which was prepared as described in Example 1. C6S-10 was prepared as generally depicted in Scheme 8 when n is 1, there is a single bond between C1 and C2, R₁ is hydrogen and R₃ is 1,1-dimethylpentyl.

A mixture of compound C6S-3 (306 mg, 1.06 mmol), glycidol (355 mg, 4.79 mmol) and TEA (130 mg, 1.28 mmol) in 30 ml of THF was refluxed for 2 days at RT. The reaction progress was monitored by TLC (35% EA in PE). Upon completion, the reaction mixture was filtrated and solvent was evaporated in vacuum. Crude oil was purified by column chromatography (30% EA in PE). Compound C6S-10 was obtained at a yield of 62%.

### c) Third Procedure

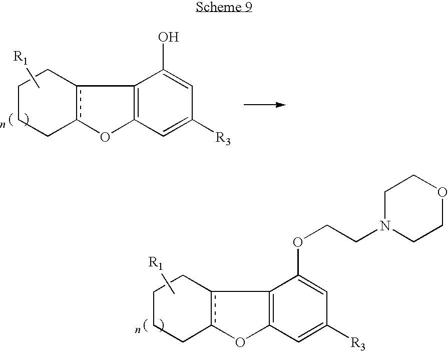

### Synthesis of Compound C7S-7: 4-{2-[2-(1,1-Dimethylheptyl)-5,6,7,8,9,9a-hexahydro-4bH-10-oxa-benzo[α]azulen-4-yloxy]-ethyl}-morpholine

The synthesis of compound C7S-7 is based on the eterification of compound C7S-1, which was prepared as described in Example 1. C7S-7 was prepared as generally depicted in Scheme 9 when n is 2, there is a single bond between C1 and C2, R₁ is hydrogen and R₃ is 1,1-dimethylheptyl.

C7S-1 was dissolved in ACN containing solid Cs₂CO₃, and refluxed while stirring for 2 hrs. 4-(2-Chloro-ethyl)-morpholine hydrochloride was then added in one portion to the reaction mixture which was stirred at reflux for an additional 3 hrs under a N₂ atmosphere. The progress of the reaction was monitored by TLC. Ethyl acetate (30 ml) was added to the mixture, which was washed three times with brine. The organic layer was dried over Na₂SO₄ (anh.), filtered through a silica bed (eluent: EtOAc) and evaporated under reduced pressure to afford 155 mg of clean C7S-7 which was used without further purification

According to a similar method, using different starting materials, the following compounds were obtained.
C6S-13 4-{2-[3-(1,1-dimethylpentyl)-5a,6,7,8,9,9a-hexahydro-dibenzofuran-1-yloxy]-ethyl}-morpholine
C7S-18 2-(1,1-dimethylheptyl)-4-(2-morpholin-4-yl-ethoxy)-5,6,7,8,9,9a-hexahydro-4bH-10-oxa-benzo[α]azulen-9-ol

### d) Fourth Procedure

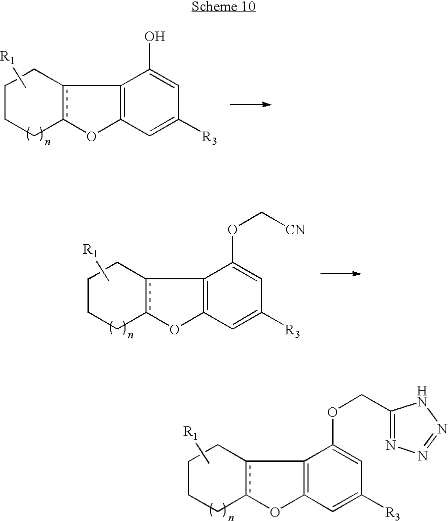

### Synthesis of Compound C6S-22: 5-[3-(1,1-Dimethylpentyl)-5a,6,7,8,9,9a-hexahydro-dibenzofuran-1-yloxymethyl]-1H-tetrazole

C6S-22 was prepared as generally depicted in Scheme 10 when n is 1, there is a single bond between C1 and C2, R₁ is hydrogen and R₃ is 1,1-dimethylpentyl The starting benzofuran is C6S-3, which was prepared as described in Example 1.

C6S-3 (0.23 g) was dissolved in ACN (50 ml) containing solid Cs₂CO₃ (0.5 g), and refluxed while stirring for 2 hrs. Chloroacetonitrile (0.5 ml) was then added to the reaction mixture which was stirred at reflux overnight. Ethyl ether was added to the mixture, which was washed with HCl (IN) and three times with brine. The organic layer was dried over Na₂SO₄ (anh.), filtered and evaporated to afford 0.181 g of the nitrile derivative after flash chromatography (7% EA in PE).

The above afforded nitrile derivative (0.15 g), NaN₃ (0.059 g) and ZnBr₂ (0.052 g) were dissolved in isopropanol (IPA) (5 ml) and water (2 ml) The reaction mixture was stirred at reflux overnight. Ethylacetate and HCl 1N were added and stirring continued until no solid was present. The organic layer was isolated and the water phase was extracted twice with EtOAc. The combined organic layers were dried over Na₂SO₄, filtered and evaporated. 165 mg of C6S-22 were obtained.

According to a similar method, using different starting materials, C7S-19, 2-(1,1-dimethylheptyl)-4-(2H-tetrazol-5-ylmethoxy)-5,6,7,8,9,9a-hexahydro-4bH-10-oxa-benzo [α]azulen-9-ol was prepared at the yield of 47.7%.

### e) Fifth Procedure

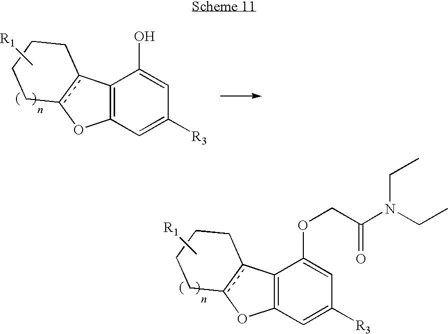

### Synthesis of Compound C7S-17: 2-[2-(1,1-Dimethylheptyl)-9-hydroxy-5,6,7,8,9,9a-hexahydro-4bH-10-oxa-benzo[ ]azulen-4-yloxy]-N,N-diethyl-acetamide

C7S-17 was prepared as generally depicted in Scheme 11 when n is 2, there is a single bond between C1 and C2, R₁ is hydroxyl and R₃ is 1,1-dimethylheptyl. The starting benzofuran is C7S-2, which was prepared as described in the first procedure of Example 3.

A solution of t-BuOK (31 mg, 0.27 mmol) in THF (dry, 3 ml) was added dropwise to a solution of C7S-2 (70 mg, 0.2 mmol) in THF (dry, 2 ml). The resulting mixture was stirred for 1 hr at RT. Bromoacetyldiethylamide (44 ml, 0.22 mmol) was added and reaction mixture was stirred overnight at RT. The progress of the reaction was monitored by TLC (25% EA in PE). Ethyl acetate was added and mixture was washed twice with 1N HCl solution and then with brine. The organic phase was dried over sodium sulfate and solvent was removed under reduced pressure. The product was purified by column chromatography (25% EA in PE). C7S-17 was obtained at a yield of 79%.

### Example 5

### Method E

### Oxidation and Oximation

### a) First Procedure

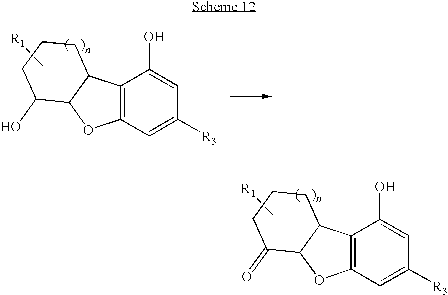

### Synthesis of Compound C7S-5: 2-(1,1-Dimethylpentyl)-4-hydroxy-4-b,5,6,7,8,9a-hexahydro-10-oxa-benzo[α]azulen-9-one

The synthesis of compound C7S-5 is based on the oxydation of the hydroxyl of C7S-4, which was prepared as described in the first procedure of Example 3, into a carbonyl. C7S-5 was prepared as generally depicted in Scheme 12 when n is 2, R₁ is hydrogen and R₃ is 1,1-dimethylpentyl.

To a mixture of C7S-4 (69 mg, 0.21 mmol) in 10 ml of pyridine, pyridinium dichromate (102 mg, 0.27 mmol) was added and the resulting mixture was stirred for 12 hrs at RT. The reaction progress was monitored by TLC. The organic solvent was then evaporated under vacuum and the crude product purified by column chromatography. Compound C7S-5 was afforded at a yield of 65%.

According to a similar method, using different starting materials, the following compounds were obtained.
C6S-25 2-(1,1-dimethylheptyl)-4-hydroxy-5,6,7,8,9,9a-hexahydro-4bH-10-oxa-benzo[α]azulen-5-carboxylic acid methyl ester
C6S-26 7-(1,1-dimethylheptyl)-9-hydroxy-2,3,4a,9b-tetrahydro-1H-dibenzofuran-4-one
C7S-6 2-(1,1-dimethylheptyl)-4-hydroxy-4-b,5,6,7,8,9a-hexahydro-10-oxa-benzo[α]azulen-9-one

### b) Second Procedure

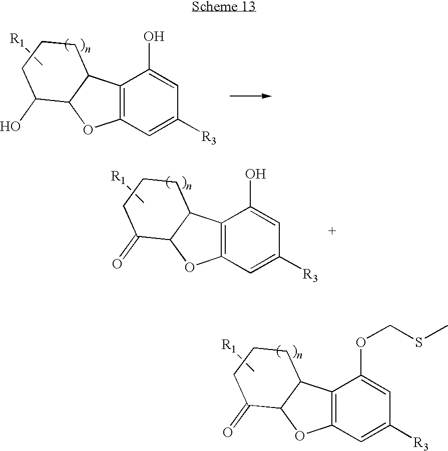

### Synthesis of Compounds C6S-28 and C6S-29: 7-(1,1-Dimethylpentyl)-9-methylsulfanylmethoxy-2,3,4a,9b-tetrahydro-1H-dibenzofuran-4-one. 7-(1,1-Dimethylpentyl)-9-hydroxy-2,3,4a,9b-tetrahydro-1H-dibenzofuran-4-one

C6S-28 and C6S-29 were prepared as generally depicted in Scheme 13 when n is 1, R₁ is hydrogen and R₃ is 1,1-dimethylpentyl. The starting benzofuran is C6S-11, which was prepared as described in the first procedure of Example 3.

A solution of C6S-11 (4,009 mg, 13.16 mmol) and DMSO (8 ml) in DCM (50 ml) was cooled down (−50° C.) while stirring under N₂. Oxalyl chloride (2,862 mg, 22.35 mmol) was added dropwise and the resulting mixture was stirred for one hr. Triethylamine (10 ml) was added and mixture was stirred overnight at RT. The mixture was washed with 1N HCl and brine. After drying the organic phase over sodium sulfate, the solvent was removed under reduced pressure and the resulting crude (oil, 3,410 mg) was purified by column chromatography (25% EA in PE). Two fractions were obtained. The isolated compounds were characterized by MS and ¹H-NMR. C6S-28 was obtained as the first fraction at a yield of 29% and C6S-29 was obtained as the second fraction at a yield of 26%.

According to a similar method, using different starting materials, the following compounds were obtained.
C6S-32 7-(1,1-dimethylpentyl)-9-hydroxy-3,3-dimethyl-2,3,4a,9b-tetrahydro-1H-dibenzofuran-4-one
C6S-33 7-(1,1-dimethylpentyl)-3,3-dimethyl-9-methylsulfanylmethoxy-2,3,4a,9b-tetrahydro-1H-dibenzo furan-4-one

### c) Third Procedure

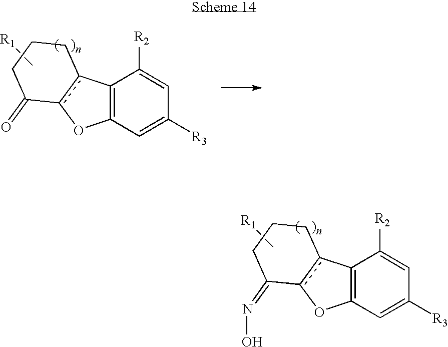

### Synthesis of Compound C6S-27: 7-(1,1-Dimethylheptyl)-9-hydroxy-2,3,4a,9b-tetrahydro-1H-dibenzofuran-4-one oxime

C6S-27 was prepared as generally depicted in Scheme 14 when n is 1, there is a single bond between C1 and C2, R₁ is hydrogen, R₂ is hydroxyl and R₃ is 1,1-dimethylheptyl The starting benzofuran is C6S-26, which was prepared as described in the first procedure of Example 5.

The mixture of C6S-26 (50 mg, 0.15 mmol), hydroxylamine hydrochloride (160 mg, 2.3 mmol) and sodium acetate (252 mg, 3.07 mmol) in ethanol (10 ml) was refluxed overnight. Ethyl acetate was added and mixture was washed with water and brine. After drying end evaporating off solvent crude oil was purified by column chromatography (25% EA in PE). C6S-27 was obtained at a yield of 85%.

According to a similar method, using different starting materials, the following compounds were obtained.
C6S-30 7-(1,1-dimethylpentyl)-9-methylsulfanylmethoxy-2,3,4a,9b-tetrahydro-1H-dibenzofuran-4-one oxime
C6S-31 7-(1,1-dimethylpentyl)-9-hydroxy-2,3,4a,9b-tetrahydro-1H-dibenzofuran-4-one oxime
C6S-34 7-(1,1-dimethylpentyl)-9-hydroxy-3,3-dimethyl-2,3,4a,9b-tetrahydro-1H-dibenzofuran-4-one oxime
C6S-35 7-(1,1-dimethylpentyl)-3,3-dimethyl-9-methylsulfanylmethoxy-2,3,4a,9b-tetrahydro-1H-dibenzofuran-4-one oxime
C7S-25 2-(1,1-dimethylheptyl)-4-hydroxy-4b,5,6,7,8,9a-hexahydro-10-oxa-benzo[α]azulen-9-one oxime

### Example 6

### Method F

### Nitration

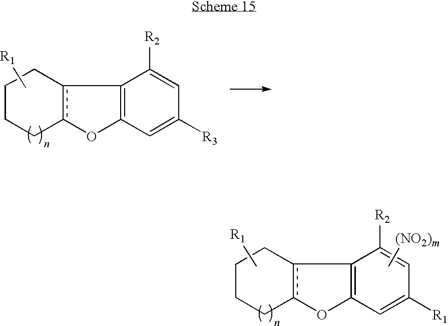

### Synthesis of Compounds C6SN-1, C6SN-2, and C6SN-3: 3-(1,1-Dimethylpentyl)-2,4-dinitro-5a,6,7,8,9,9a-hexahydro-dibenzofuran-1-ol, 3-(1,1-Dimethylpentyl)-2-nitro-5a,6,7,8,9,9a-hexahydro-dibenzofuran-1-ol and 3-(1,1-Dimethylpentyl)-4-nitro-5a,6,7,8,9,9a-hexahydro-dibenzofuran-1-ol, respectively.

The synthesis of compound C6SN-1 is as depicted in Scheme 15 when n is 1, there is a single bond between C1 and C2, R₁ is hydrogen, R₂ is hydroxyl, R₃ is 1,1-dimethylpentyl and the benzene ring is substituted with nitro groups at both positions 2 and 4, the hydroxyl group being at position 1. C6SN-2 is mono-substituted with a nitro group at position 2, whereas C6SN-3 is mono-substituted with a nitro group at position 4. All three compounds were prepared basically following the same synthetic procedure and separated. The starting benzofuran is C6S-3, which was prepared as described in Example 1.

A suspension of 250 mg of C6S-3 in 2 ml of HNO₃ was heated to reflux. To the hot suspension were added 500 μl of acetic anhydride and 100 μl of acetic acid. After 15 minutes the reaction was quenched by the addition of water. The dark red solution was extracted several times with EA, and the combined organic fractions were dried over sodium sulfate. Removal of the solvent under reduced pressure, afforded 300 mg of compound C6SN-1 as a yellow oil.

To a stirred solution of 250 mg of C6S-3 in 500 μl of acetic anhydride cooled to 0° C. were added 35 μl of nitric acid dissolved in 100 μl of acetic acid. The reaction was allowed to warm slowly and was left to stir at room temperature for a period of 18 hrs. After quenching by the addition of water, the reaction solution was extracted several times with ethyl acetate, and the combined organic fractions were dried over sodium sulfate. Following removal of the solvent under reduced pressure, the crude product was purified with column chromatography (10% EA in PE) resulting in four fractions. The second and third fractions afforded 75 mg and 123 mg of C6SN-2 and C6SN-3, respectively.

According to a similar method, using different starting materials, the following compounds were obtained.
C7SN-1 2-(1,1-dimethylheptyl)-3-nitro-5,6,7,8,9,9a-hexahydro-4bH-10-oxa-benzo[α]azulen-4,9-diol
C7SN-2 2-(1,1-dimethylheptyl)-1-nitro-5,6,7,8,9,9a-hexahydro-4bH-10-oxa-benzo[α]azulen-4,9-diol

### Example 7

### Method G

### Iodocyclization and Dehydroiodination

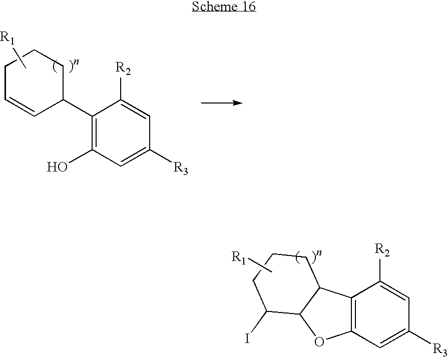

### Synthesis of compound C7S-26: 2-(1,1-Dimethylheptyl)-9-iodo-5,6,7,8,9,9a-hexahydro-4bH-10-oxa-benzo[α]azulen-4-ol

C7S-26 was prepared as generally depicted in Scheme 16 when n is 2, R₁ is hydrogen, R₂ is hydroxyl and R₃ is 1,1-dimethylheptyl.

A solution of iodine (610 mg, 2.4 mmol) in 100 ml of ACN was added dropwise to a mixture of 2-Cyclohept-2-enyl-3-(1′,1′-dimethylheptyl)-benzene-1,5-diol (533 mg, 1.61 mmol) and sodium carbonate (2,820 mg, 26.6 mmol) in 50 ml of ACN. The mixture was stirred overnight at RT. Ethyl acetate was added and mixture was washed with water and brine. After drying end evaporating off solvent the crude oil was purified by column chromatography (10% EA in PE). C7S-26 was obtained at a yield of 71%.

Similarly, two compounds were prepared wherein n is 1 and R₃ is either 1,1-dimethylheptyl or 1,1-dimethylpentyl, namely 3-(1,1-dimethylheptyl)-6-iodo-5a,6,7,8,9,9a-hexahydro-dibenzofuran-1-ol and 3-(1,1-dimethylpentyl)-6-iodo-5a,6,7,8,9,9a-hexahydro-dibenzofuran-1-ol. They were directly dehydroiodinated as described below for compound C6S-2 and as generally depicted in Scheme 17 when n is 1, R₁ is hydrogen, R₂ is hydroxyl and R₃ is 1,1-dimethylpentyl.

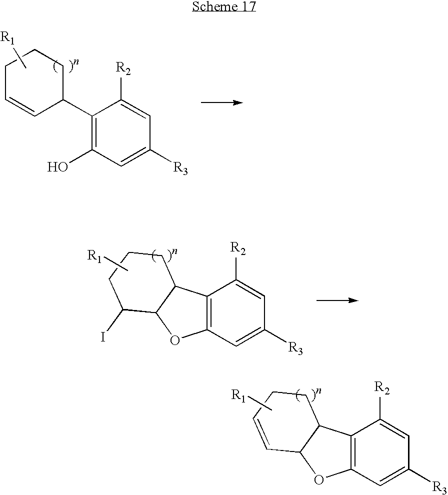

### Synthesis of Compound C6S-2: 3-(1,1-Dimethylheptyl)-5a,8,9,9a-tetrahydro-dibenzofuran-1-ol

A mixture of 3-(1,1-dimethylheptyl)-6-iodo-5a,6,7,8,9,9a-hexahydro-dibenzofuran-1-ol (77 mg, 0.17 mmol) and sodium acetate (150 mg, 1.82 mmol) in 10 ml of dry DMF was stirred for about 12 hrs under heating conditions (80-85° C.). The reaction progress was monitored by TLC. The reaction mixture was washed twice with sodium bicarbonate solution and then with brine. After phase separation and evaporation of the organic phase the product was isolated and purified by column chromatography. Compound C6S-2 was obtained at a yield of 86%.

Using a similar method, compound C6S-4 was prepared at a yield of 79%. Also prepared was 2-(1,1-dimethylheptyl)-5,6,7,9a-tetrahydro-4bH-10-oxa-benzo[α]azulen-4-ol at a yield of 86%.

Such compounds having a cycloalkenic fused ring can be further reacted to give diol derivatives as described below for compound C7S-11 and as generally depicted in Scheme 18 when n is 2, R₁ is hydrogen, R₂ is hydroxyl and R₃ is 1,1-dimethylheptyl.

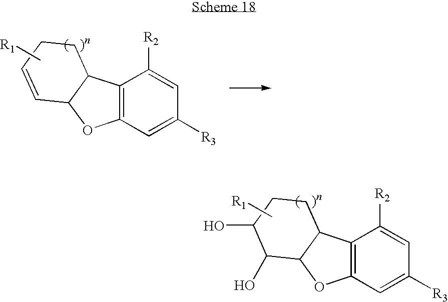

### Synthesis of Compound C7S-11: 2-(1,1-Dimethylheptyl)-5,6,7,8,9,9a-hexahydro-4bH-10-oxa-benzo[α]azulen-4,8,9-triol

2-(1,1-Dimethylheptyl)-5,6,7,9a-tetrahydro-4bH-10-oxa-benzo[α]azulen-4-ol (104 mg, 0.31 mmol) was dissolved in acetone and the solution was cooled with ice. Solution of osmium tetraoxide was added in one portion under N₂ and the mixture was stirred ON. Water was added and the mixture was washed with sodium bisulfite and extracted with ethyl acetate. The organic layer was washed twice with brine and dried over sodium sulfate. After removing of solvent, the crude oil was purified by column chromatography (15% IPA in hexane). Compound C7S-11 was obtained at a yield of 31%.

### Example 8

### Method H

### Oxidation

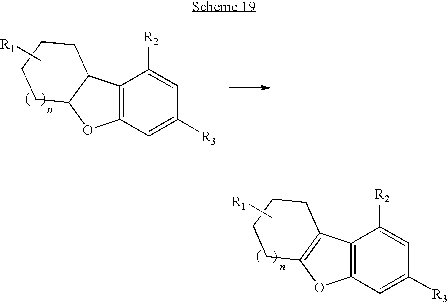

### Synthesis of Compound C6M-10: 3-(1,1-Dimethylheptyl)-7,7-dimethyl-6,7,8,9-tetrahydro-dibenzofuran-1-ol

C6M-10 was prepared as generally depicted in Scheme 19 when n is 1,R₁ is gem-dimethyl, R₂ is OH and R₃ is 1,1-dimethylheptyl. The starting benzofuran is C6S-17, which was prepared as described in Example 1.

A solution of C6S-17 (144 mg, 0.42 mmol) and 2,3-dichloro-5,6-dicyano-p-benzoquinone (100 mg, 0.44 mmol) in dichloroethane (25 ml) was heated to 80° C. for 2.5 hrs. Another 0.5 eq. of 2,3-dichloro-5,6-dicyano-p-benzoquinone (50 mg, 0.22 mmol) was added to the reaction, and the reaction allowed to stir for an additional hour at 80° C. The reaction solution was filtered through celite. The 1,2-dichloroethane was removed under vacuum and the residue redissolved in methylene chloride. The solution was refiltered to remove any undissolved material. The crude material was purified by column chromatography to afford pure C6M-10 at a yield of 12%.

### Example 9

### Method J

### Dithiane Deprotection

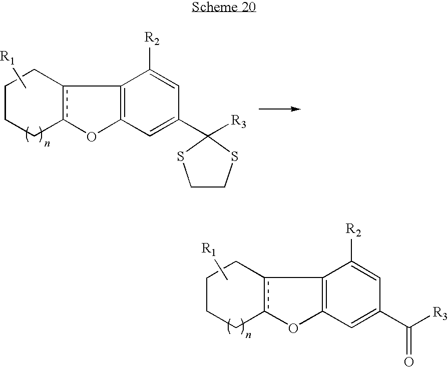

### Synthesis of Compound C6S-20:1-(1-Hydroxy-5a,6,7,8,9,9a-hexahydro-dibenzofuran-3-yl)-ethanone

C6S-20 was prepared as generally depicted in Scheme 20 when n is 1, there is a single bond between C1 and C2, R₁ is hydrogen, R₂ is hydroxyl and R₃ is methyl. The starting benzofuran is C6S-12, which was prepared as described in Example 1.

A solution of AgNO₃ (0.82 g, 4.83 mmol) in water (3 ml) was added to a stirred solution of C6S-12 (0.5 g, 1.62 mmol) in ethanol (20 ml) at RT. The reaction mixture was stirred at RT overnight. After filtration, the solution was diluted in EtOAc, washed with brine (×2) and dried (Na₂SO₄ anh.). Solvent evaporation afforded 315 mg of a crude material that was recrystallized from ACN yielding 162 mg of clean C6S-20.

### Example 10

### Method K

### Acylation

### a) First Procedure

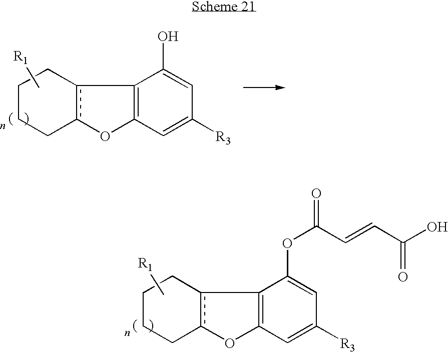

### Synthesis of Compound C6M-2: But-2-enedioic acid mono-[3-(1,1-dimethylheptyl)-6,7,8,9-tetrahydro-dibenzofuran-1-yl]ester

Compound C6M-2 was prepared as depicted in Scheme 21 when n is 1, there is a double bond between C1 and C2, R₁ is hydrogen and R₃ is 1,1-dimethylheptyl. The starting benzofuran is C6M-1, which was prepared as described in the first procedure of Example 2.

To a stirred solution of C6M-1 (0.3 g, 0.96 mmol) in 10 ml of anhydrous THF at −40° C. under a N₂ atmosphere was added fumaryl chloride (0.13 ml, 1.5 mmol) dropwise by syringe immediately followed by triethylamine (0.160 ml, 1.5 mmol). After complete addition, the reaction was removed from the cold bath and warmed to room temperature followed by an additional 1 hr of stirring. The reaction was quenched by the addition of water, followed by extraction with diethyl ether. The combined organic fractions were washed with water, dried over sodium sulfate and the solvent removed under reduced pressure, affording 530 mg of crude product. Purification with column chromatography afforded pure C6M-2 at a yield of 76%.

According to a similar method, using different starting materials, the following compounds were obtained.
C5S-5 But-2-enedioic acid mono-[6-(1,1-dimethylheptyl)-1,8a-dimethyl-2,3,3a,8a-tetrahydro-1H-8-oxa-cyclopenta[α]inden-4-yl]ester
C6S-14 But-2-enedioic acid mono-[3-(1,1-dimethylpentyl)-5a,6,7,8,9,9a-hexahydro-dibenzofuran-1-yl]ester
C6S-37 But-2-enedioic acid mono-[3-(1,1-dimethylheptyl)-7,7-dimethyl-5a,6,7,8,9,9a-hexahydro-dibenzofuran-1-yl]ester
C6S-40 But-2-enedioic acid mono-[7,7-dimethyl-3-(2-phenethyl-[1,3]dithiolan-2-yl)-5a,6,7,8,9,9a-hexahydro-dibenzofuran-1-yl]ester
C6M-8 But-2-enedioic acid mono-[3-(1,1-dimethylpentyl)-6,7,8,9-tetrahydro-dibenzofuran-1-yl]ester
C7S-9 But-2-enedioic acid mono-[2-(1,1-dimethylheptyl)-5,6,7,8,9,9a-hexahydro-4bH-10-oxa-benzo[α]azulen-4-yl]ester
C7S-12 But-2-enedioic acid mono-[9-(3-carboxy-acryloyloxy)-2-(1,1-dimethylheptyl)-5,6,7,8,9,9a-hexahydro-4bH-10-oxa-benzo[α]azulen-4-yl]ester
C7S-28 But-2-enedioic acid mono-[2-(1,1-dimethylheptyl)-9-iodo-5,6,7,8,9,9a-hexahydro-4bH-10-oxa-benzo[α]azulen-4-yl]ester

### b) Second Procedure

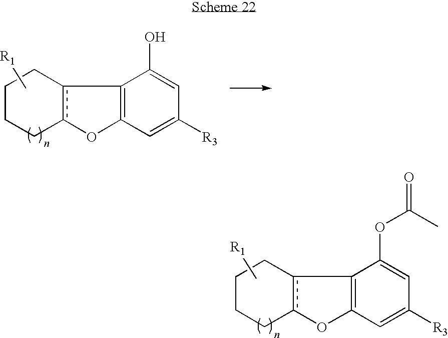

### Synthesis of Compound C6S-15: Acetic acid 3-(1,1-dimethylpentyl)-5a,6,7,8,9,9a-hexahydro-dibenzofuran-1-yl ester

Compound C6S-15 was prepared as depicted in Scheme 22 when n is 1, there is a single bond between C1 and C2, R₁ is hydrogen and R₃ is 1,1-dimethylpentyl. The starting benzofuran is C6S-3, which was prepared as described in Example 1.

A mixture of C6S-3 (0.15 g) and acetic anhydride (3 ml) in 3 ml pyridine was stirred for 4 hrs at RT under N₂. The reaction mixture was then poured over crushed ice and extracted with Et₂O (3×60 ml). The combined organic layers were washed with 1N HCl (5×30 ml), water (3×30 ml) and brine (2×30 ml). After drying over MgSO₄ (anh.), the etheric solution was filtered and the solvent was evaporated under reduced pressure yielding 0.152 g of clean solid C6S-15.

### c) Third Procedure

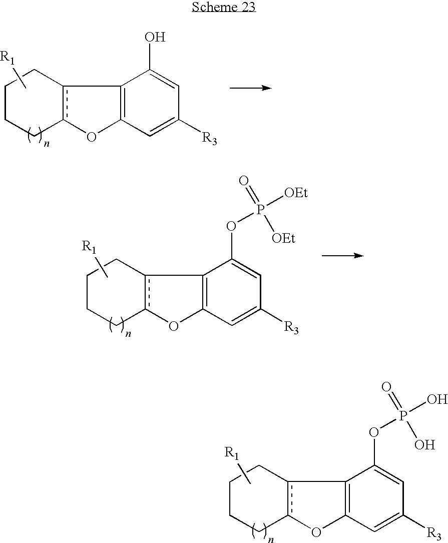

### Synthesis of Compound C6S-16: Phosphoric acid mono-[3-(1,1-dimethylpentyl)-5a,6,7,8,9,9a-hexahydro-dibenzofuran-1-yl]ester

Compound C6S-16 was prepared as depicted in Scheme 23 when n is 1, there is a single bond between C1 and C2, R₁ is hydrogen and R₃ is 1,1-dimethylpentyl.

Diethyl phosphoric acid mono-[3-(1,1-dimethylpentyl)-5a,6,7,8,9,9a-hexahydro-dibenzo-furan-1-yl]ester (1) To a stirred solution of C6S-3 (0.76 mmol) in dry THF (15 ml) was added Potassium t-butoxide (1 mmol). After 15 min of stirring under N₂ at RT, the phosphorochloridic acid diethyl ester was added, and the reaction was left to stir at RT for a period of 18 hrs. After removal of the THF, the residue was redissolved in EtOAC and was washed with water (×2) and brine (×2). The organic solution was dried over sodium sulfate and the solvent removed affording 374 mg of crude phosphate ester (1).

To a solution of the previously prepared phosphate ester 1 (0.76 mmol) dissolved in dry methylene chloride (4 Å molecular sieves) (10 ml) was added bis(trimethylsilyl)trifluoroacetamide (7.6 mmol), and the reaction was stirred under N₂ at RT for 20 min. The reaction solution was cooled to 0° C. in an ice water bath, and to the cooled solution trimethylsilyl iodide (6.1 mmol) was introduced. The reaction was stirred at 0° C. for 1 hr, whereupon the bath was removed and the reaction stirred at RT. After an additional 2 hrs of stirring, the supernatant solvent was removed and the residue dissolved in a mixture of 10:5:3 ACN:H₂O:trifluoroacetic acid (18 ml). After 1 hr, the solvent was removed and the brown residue lyophilized to afford 165 mg of C6S-16 as a brown powder.

According to a similar method, C7S-13, Phosphoric acid mono-[2-(1,1-dimethylheptyl)-9-hydroxy-5,6,7,8,9,9a-hexahydro-4bH-10-oxa-benzo[α]azulen-4-yl]ester, was prepared.

### d) Fourth Procedure

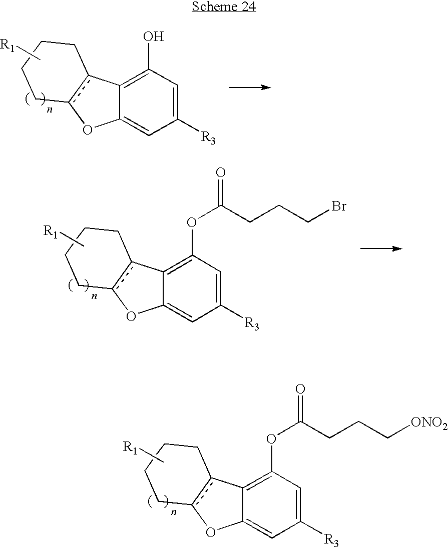

### Synthesis of Compound C6S-24: 4-Nitrooxy-butyric acid 3-(1,1-dimethylpentyl)-5a,6,7,8,9,9a-hexahydro-dibenzofuran-1-yl ester

Compound C6S-24 was prepared as depicted in Scheme 24 when n is 1, there is a single bond between C1 and C2, R₁ is hydrogen and R₃ is 1,1-dimethylpentyl. The starting benzofuran is C6S-3, which was prepared as described in Example 1.

C6S-3 (0.11 g) was dissolved in dry THF (10 ml) containing TEA (0.1 ml). 4-Bromo-butyryl chloride (0.15 ml in 1 ml THF) was then added dropwise to the mixture, which was stirred at RT for 3-4 hrs under N₂. Ethyl acetate (100 ml) and water were added to the mixture, which was washed with NaHCO₃, brine and water. The organic layer was dried over Na₂SO₄ (anh.), filtered and evaporated under reduced pressure to afford 320 mg of a crude 4-bromobutyric acid 3-(1,1-dimethylpentyl)-5a,6,7,8,9,9a-hexahydro-dibenzofuran-1-yl ester that was used without further purification. This crude material was dissolved in 10 ml of CH₃CN (dry) and silver nitrate (0.2 g) was added. The reaction mixture was refluxed at RT for 4 hrs. The reaction progress was followed by TLC and HPLC. An additional portion of silver nitrate (0.2 g) was added and the mixture was further refluxed overnight. At reaction completion, activated charcoal was added and the mixture was filtered through a silica bed. The solvent was then removed under reduced pressure Following dilution in EtOAc, the mixture was washed with water and brine (×2). The organic phase was dried over Na₂SO₄ and the solvent evaporated yielding 205 mg of a crude material. C6S-24 (yield: 63 mg) was purified via biotage chromatography (5% EA in PE)

### e) Fifth Procedure

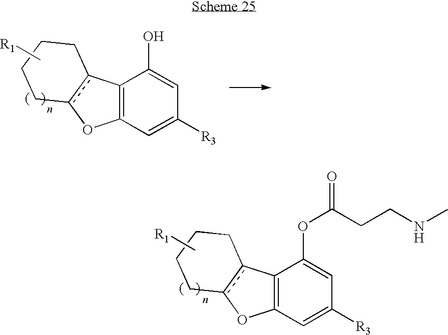

### Synthesis of Compound C6S-39: 3-Methylamino-propionic acid 3-(1,1-dimethylheptyl)-7,7-dimethyl-5a,6,7,8,9,9a-hexahydro-dibenzofuran-1-yl ester

Compound C6S-39 was prepared as depicted in Scheme 25 when n is 1, there is a single bond between C1 and C2, R₁ is gem-dimethyl and R₃ is 1,1-dimethylheptyl. The starting benzofuran is C6S-17, which was prepared as described in Example 1.

3-Piperidin-1-yl-propionic acid (0.1 g) was dissolved in dry SOCl₂ (0.5 ml) and the reaction mixture was stirred at RT under a N₂ atmosphere for 18 hrs. The unreacted SOCl₂ was carefully evaporated. A solution of C6S-17 (100 mg) and diisopropyl ethyl amine (120 mg) in dry THF (5 ml) was then added to the mixture and stirring at RT continued for 24 hrs. Ethyl acetate (100 ml) and water were added to the mixture which was washed with NaHCO₃, brine and water. The organic layer was dried over Na₂SO₄ (anh.), filtered and evaporated under reduced pressure. The compound was purified using medium pressure flash chromatography CombiFlash® (eluent: 20 min. linear gradient from 100% PE to 100% diethylether) and 22 mg of C6S-39 were obtained.

### f) Sixth Procedure

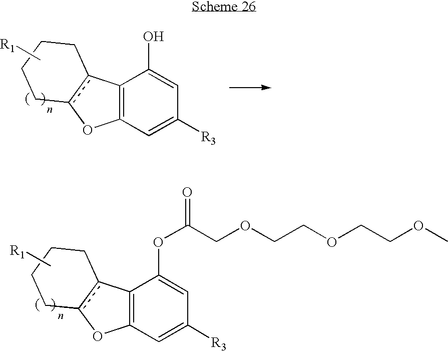

### Synthesis of Compound C7S-27: [2-(2-Methoxy-ethoxy)-ethoxy]-acetic acid 2-(1,1-dimethylheptyl)-9-hydroxy-5,6,7,8,9,9a-hexahydro-4bH-10-oxa-benzo[α]azulen-4-yl ester

Compound C7S-27 was prepared as depicted in Scheme 26 when n is 2, there is a single bond between C1 and C2, R₁ is hydroxyl and R₃ is 1,1-dimethylheptyl. The starting benzofuran is C7S-2, which was prepared as described in the first procedure of Example 3.

C7S-2 (0.15 g) was dissolved in dry DCM (30 ml) containing N,N-Dimethylaminopyridine (DMAP) (20 mg) and [2-(2-Methoxy-ethoxy)-ethoxy]-acetic acid (53 mg) the reaction mixture was stirred at RT under a N₂ atmosphere until everything dissolved. A solution of 1,3-Dicyclohexylcarbodiimide (DCC, 167 mg) in DCM (5 ml) was then added to the mixture and stirring at RT continued for 24 hrs. Ethyl acetate (100 ml) and water were added to the mixture which was washed with NaHCO₃, brine and water. The organic layer was dried over Na₂SO₄ (anh.), filtered and evaporated under reduced pressure. The compound was purified via CombiFlash chromatography as previously described and 10 mg of C7S-27 were obtained.

### g) Seventh Procedure

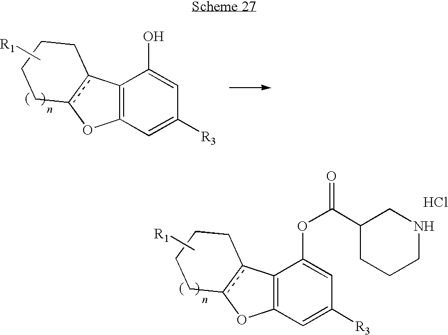

### Synthesis of Compound C6S-23: Piperidine-3-carboxylic acid 3-(1,1-dimethylpentyl)-5a,6,7,8,9,9a-hexahydro-dibenzofuran-1-yl ester

Compound C6S-23 was prepared as depicted in Scheme 27 when n is 11, there is a single bond between C1 and C2, R₁ is hydrogen and R₃ is 1,1-dimethylpentyl. The starting benzofuran is C6S-3, which was prepared as described in Example 1.

A solution of C6S-3 (284 mg, 1.0 mmol), N-Boc-3-morpholinic acid and DMAP (12.3 mg, 0.10 mmol) in 10 ml DCM was stirred for 30 min. A solution of DCC (231 mg, 1.12 mmol) in 10 ml of DCM was added dropwise and resulting mixture was stirred for overnight. The resulting dicyclohexylurea was filtered and the solvent was removed under reduced pressure. The crude resulting oil was purified by column chromatography (10% EA in PE). The product was dissolved in DCM and 5 ml of HCl-dioxane was added. Mixture was stirred overnight, then solvent was evaporated and final compound was dried in vacuum. The HCl salt of C6S-23 was obtained at a yield of 49%.

### Example 11

### Miscellaneous Methods

### a) Hydrolysis to Carboxylic Acid

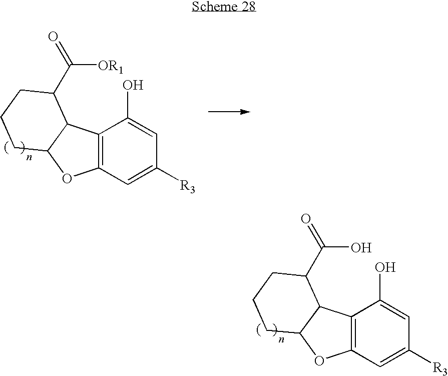

### Synthesis of Compound C7S-31: 2-(1,1-Dimethylheptyl)-4-hydroxy-5,6,7,8,9,9a-hexahydro-4bH-10-oxa-benzo[α]azulen-5-carboxylic acid

Compound C7S-31 was prepared as depicted in Scheme 28 when n is 2, R₁ is methyl and R₃ is 1,1-dimethylheptyl. The starting benzofuran is C7S-20, which was prepared as described in Example 1.

Sodium hydroxide (121 mg, 3 mmol) in 5 ml of water was added to a solution of C7S-20 (410 mg, 1.05 mmol) in methanol. The mixture was stirred overnight at RT. Ethyl acetate was added and the mixture was washed with 1N HCl and brine. After drying and evaporating off the solvent, C7S-31 was obtained as a solid at a yield of 93%.

### b) Reduction to Alcohol

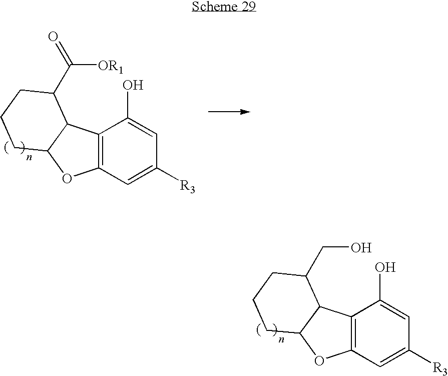

### Synthesis of Compound C7S-22: 2-(1,1-Dimethylheptyl)-5-hydroxymethyl-5,6,7,8,9,9a-hexahydro-4bH-10-oxa-benzo[α]azulen-4-ol

Compound C7S-22 was prepared as depicted in Scheme 29 when n is 2, R₁ is methyl and R₃ is 1,1-dimethylheptyl. The starting benzofuran is C7S-20, which was prepared as described in Example 1.

LiAlH₄ (1 ml, 1 M in THF) was added dropwise to a solution of C7S-20 (60 mg, 0.154 mmol) in 5 ml of THF (extra dry) and stirred for 72 hrs under a N₂ atmosphere at RT. Brine and HCl (IM) were added to the mixture and extracted three times with EtOAc. The combined organic layers were washed with brine, dried over Na₂SO₄ and filtered to afford 54 mg (yield: 100%) of C7S-22 after evaporation of the solvent under reduced pressure.

### c) Enantiomeric Separation

As explained, compounds of the invention may have at least one chiral center and therefore exist as mixtures of stereoisomers, such as enantiomers and diastereomers. Some of the compounds prepared by the above-described methods were separated into individual enantiomers using Chiral HPLC. The HPLC is performed on a chemically modified amylose-based chiral column ChiralPak AD-H, 250×4.6 mm, 5 μm particle size (Daicel Ltd). The chiral stationary phase is a tris-(3,5-dimethylphenylcarbamate) derivative of amylose immobilized on macroporous silica gel. The mobile phase was hexane:IPA and generally the chromatography was performed at RT at a flow rate of 1 ml per minute. Using this method the following compounds were separated into two fractions of individual enantiomers, F1 and F2: C6S-17, C7S-1, C7S-2 and C7S-22. The fractions of C7S-2 comprising the isolated enantiomers were named C7S-29 and C7S-30, for (−)- and (+)-2-(,1-dimethylheptyl)-5,6,7,8,9,9a-hexahydro-4bH-10-oxa-benzo[α]azulen-4,9-diol, respectively. The optical rotation of plane-polarized light of each enantiomer was determined at a concentration of 1 mg/ml in methanol using the 589 nm line of a sodium lamp of a polarimeter. Compounds C7S-23 and C7S-24 are two diastereomeric fractions of enantiomeric pairs, and they were separated into R-cis- and S-cis-[2-(1,1-dimethylheptyl)-4-hydroxy-5,6,7,8,9,9a-hexahydro-4bH-10-oxa-benzo[α]azulen-5-yl]-acetic acid methyl ester using regular column chromatography.

### d) Salification

As explained, compounds of the invention may be prepared as salt derivatives. Some compounds, such as C6S-23, could be directly obtained as salt derivatives as the result of deprotection. Other compounds could be further modified to obtain a salt thereof. For example, compounds C7S-32 and C7S-33, the synthesis of which is described in the second procedure of Example 3, were further salified using the following procedure. To a solution of C7S-32 or C7S-33 in methanol, 1N HCl was added and the resulting mixture was evaporated under reduced pressure and dried in vacuum. The resulting solid was analyzed and used without further purification.

### Example 12

### Structures and Selected Properties

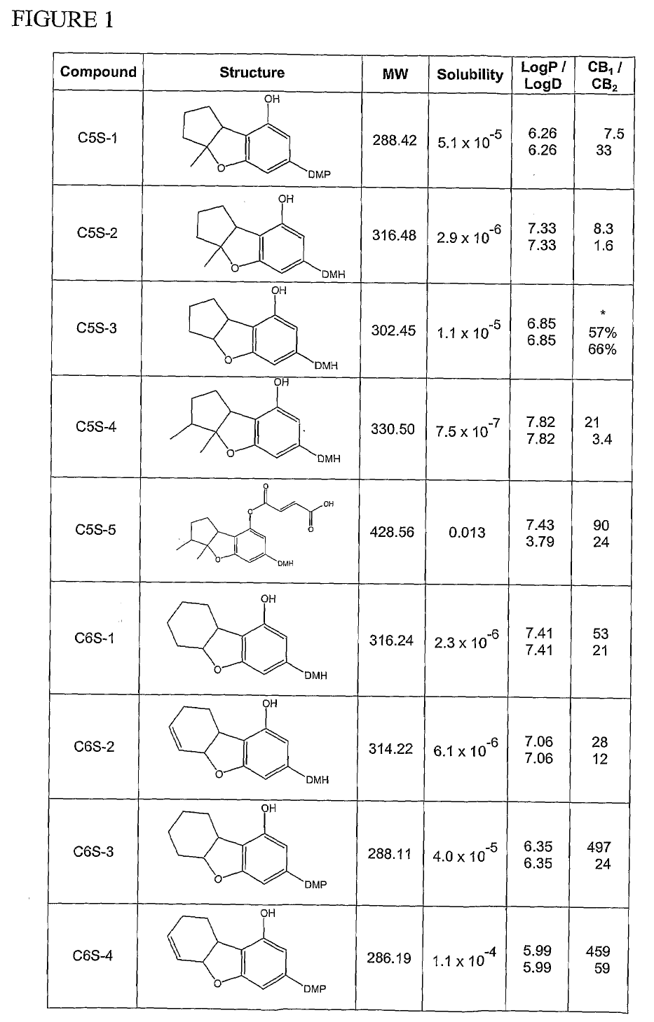

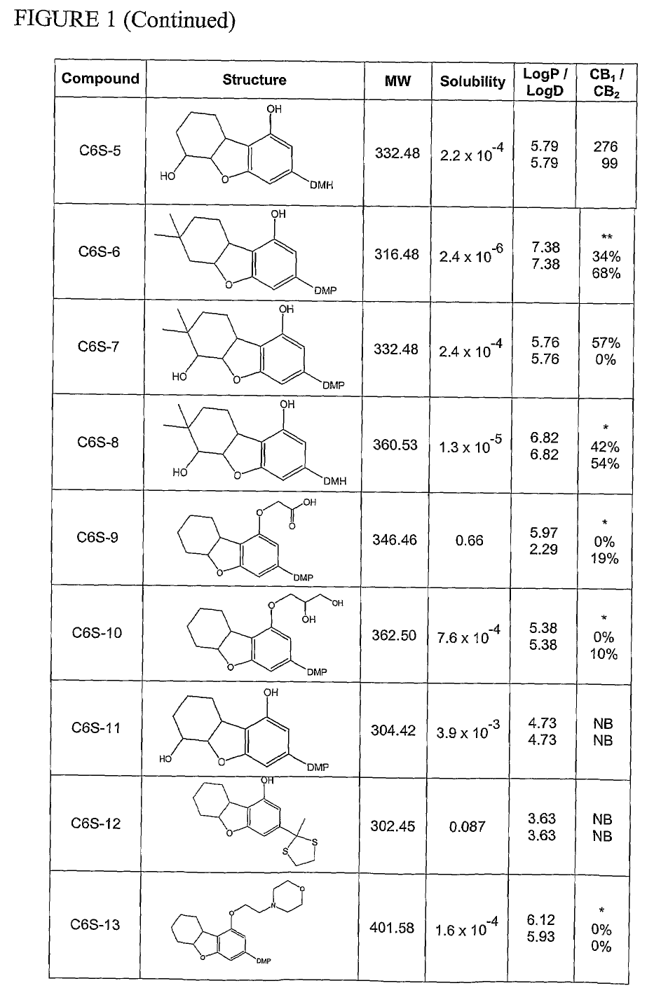

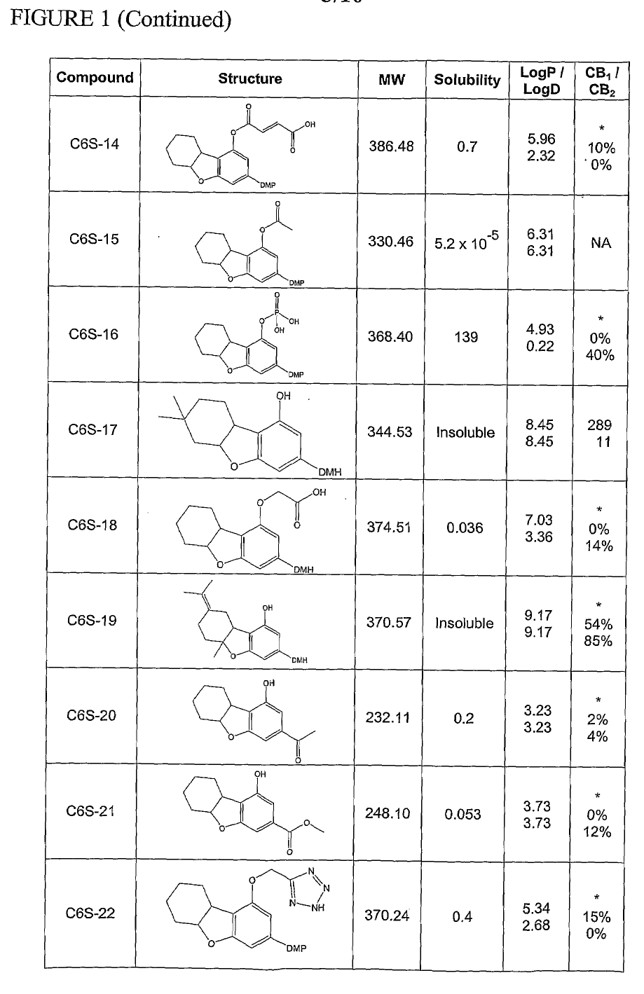

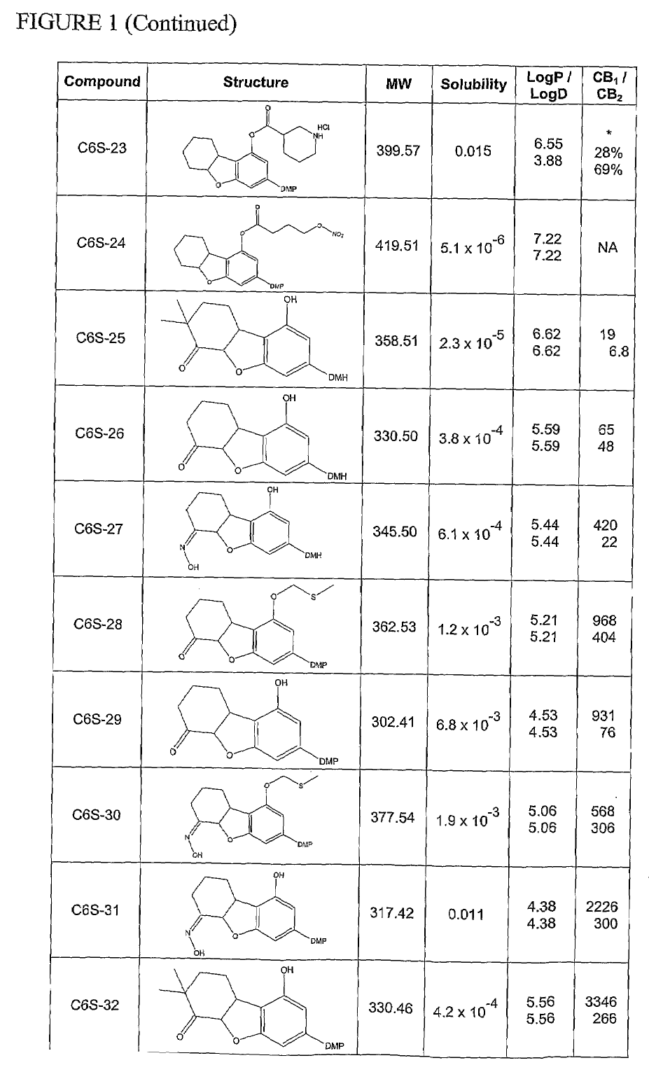

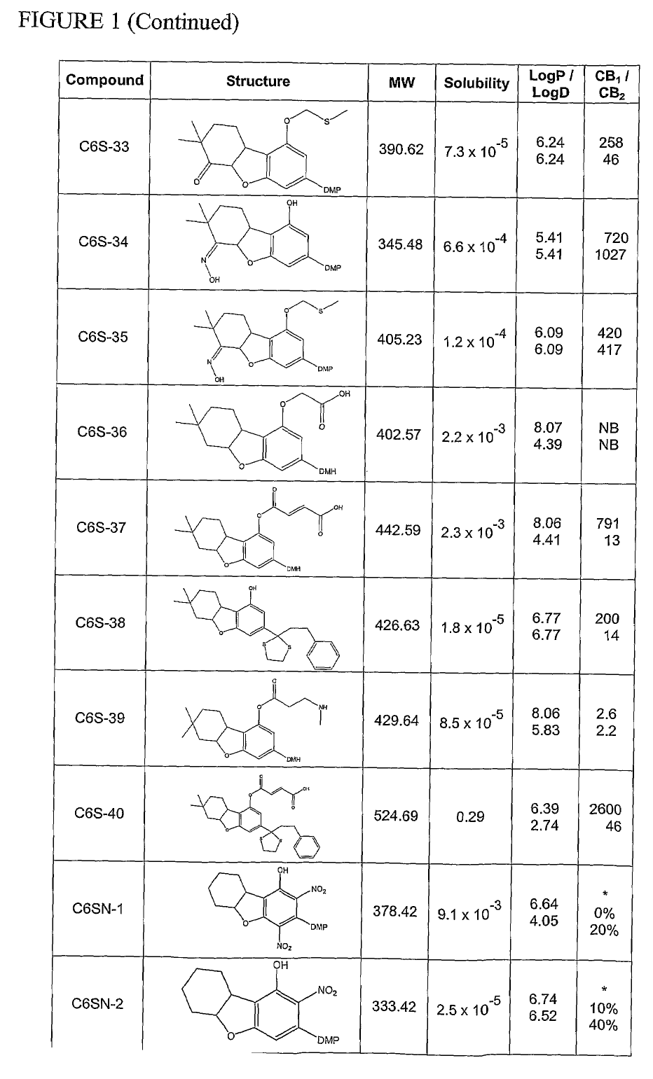

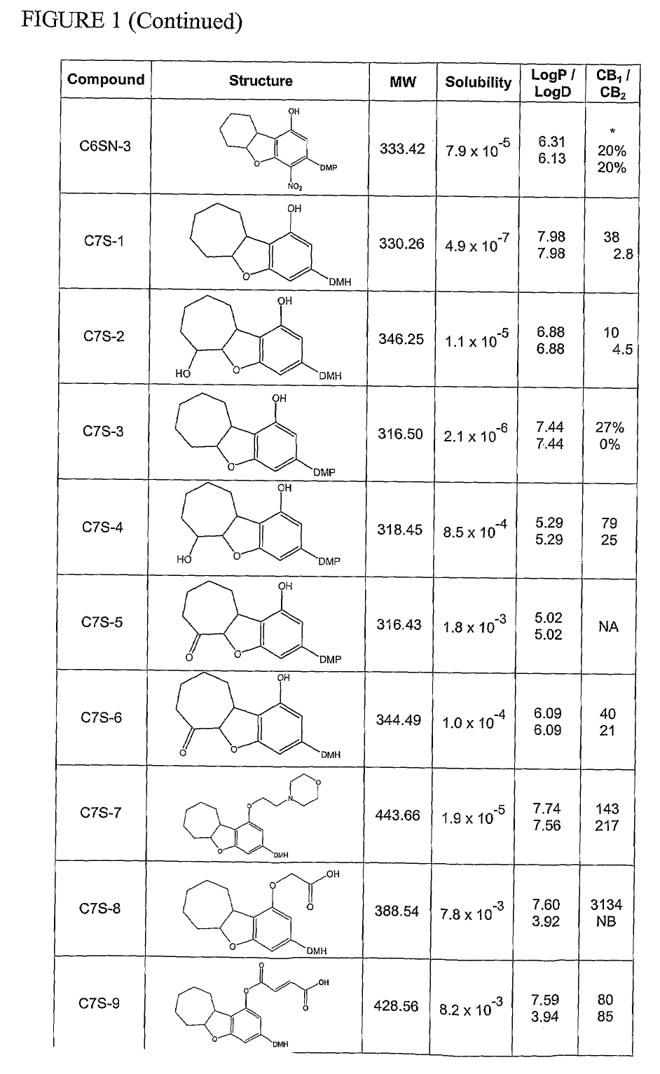

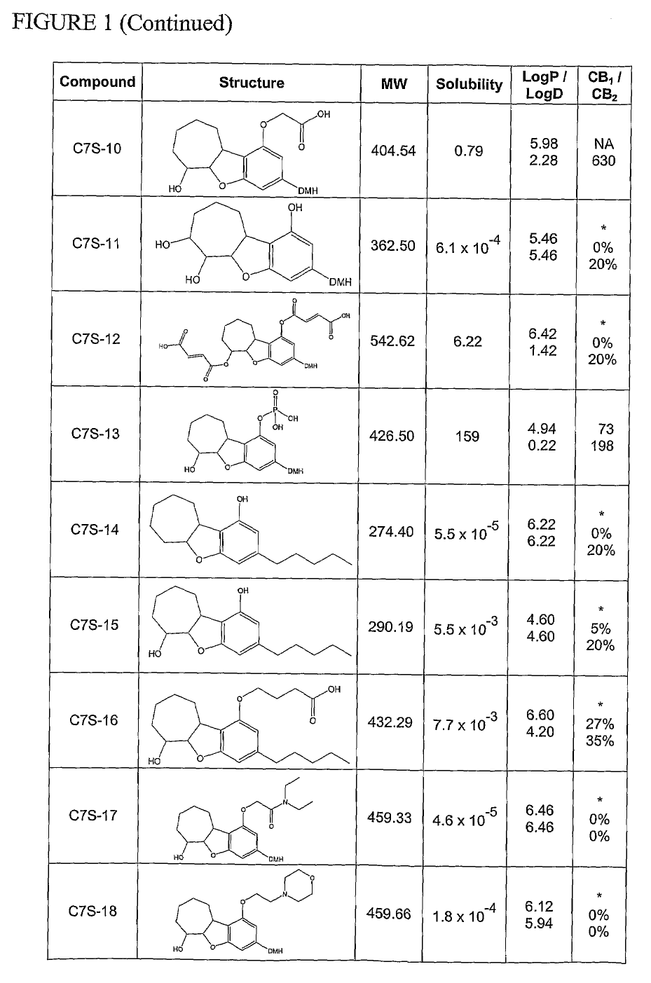

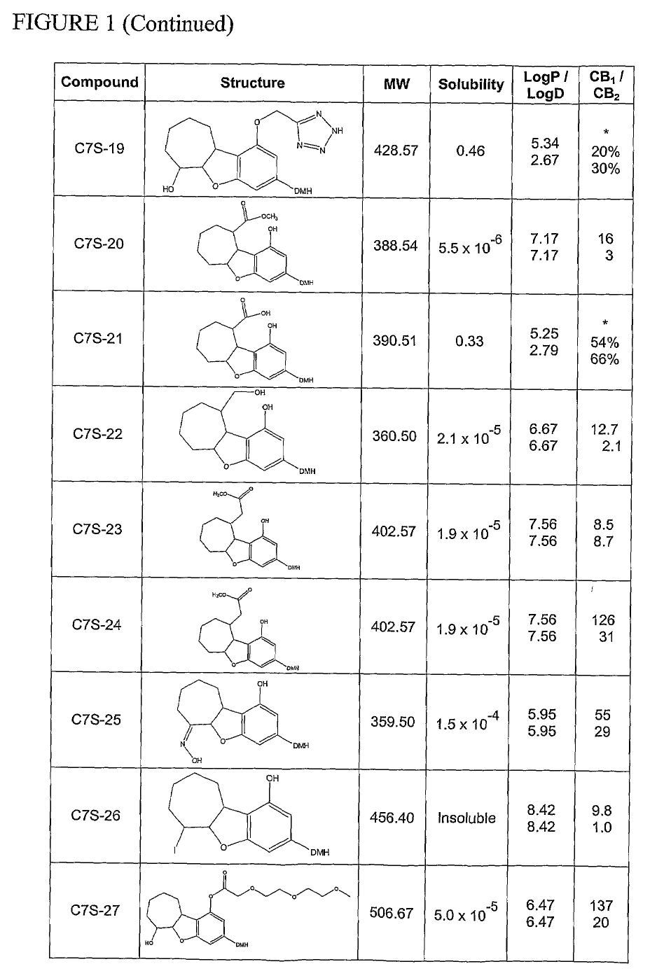

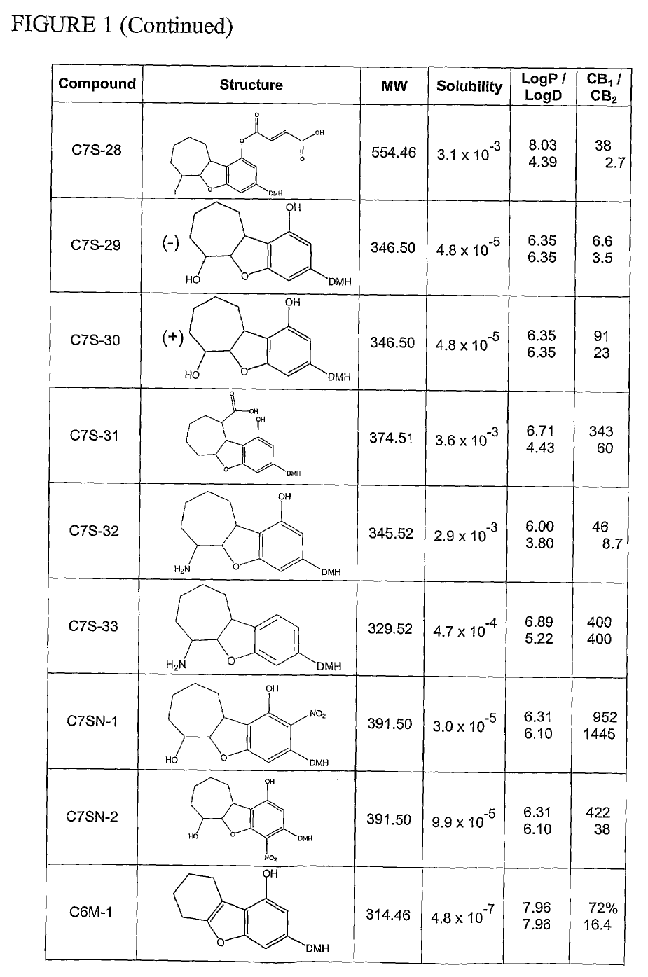

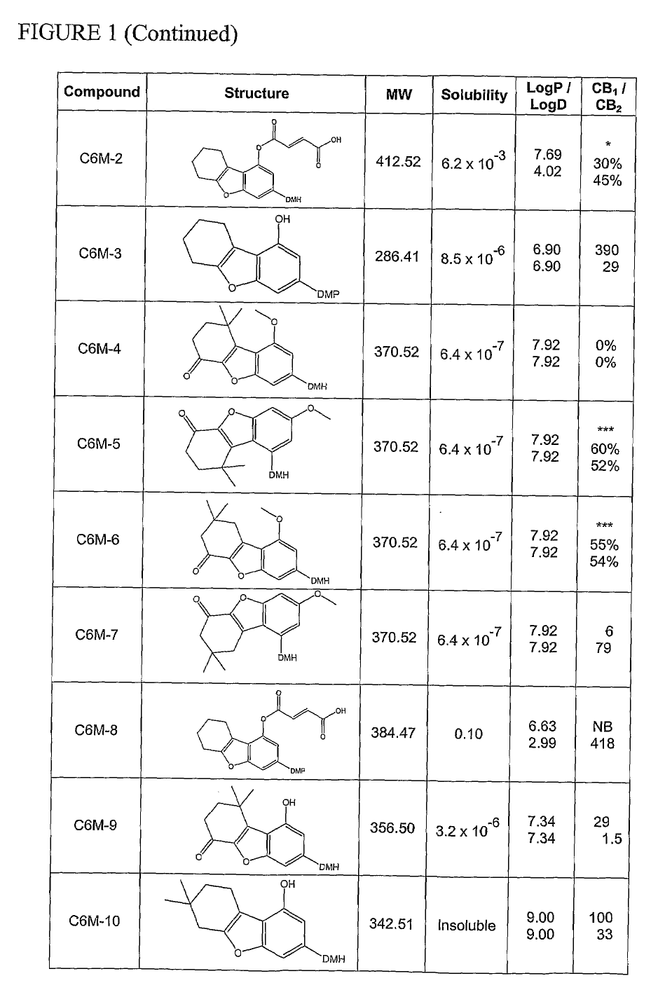

The structures of some of the compounds prepared according to the synthetic procedures disclosed above in Examples 1 through 11 are presented in tabulated form in FIG. 1. Information regarding certain physicochemical properties of these compounds is also included. Expected water solubility (g/l), logP and logD at pH 7 were calculated using Advanced Chemistry Development software (ACD labs, version 4.04). When available, the binding affinity toward the human cannabinoid receptors, expressed in Kᵢ (nM) or in percent binding at cut-off concentrations, as assayed according to Example 13 below, is indicated. The abbreviations DMP and DMH used in FIG. 1 represent a 1,1-dimethylpentyl and 1,1-dimethylheptyl group, respectively.

Evaluation of the therapeutic effects of the novel compounds of the invention was carried out in a series of experimental systems to support the utility of these drugs. Most of the techniques used to prepare the in vitro or in vivo models, testing the compounds and analyzing the outcome are widely practiced in the art, and most practitioners are familiar with the standard resource materials that describe specific conditions and procedures. However, for convenience, the following descriptions may serve as guidelines.

Unless otherwise indicated, the test compounds are prepared as follows: for in vitro assays the compounds are first dissolved and stepwise diluted in DMSO and then diluted in the assay buffer, generally tissue culture medium, down to a final concentration of 0.1% DMSO. For in vivo assays the test compounds are first diluted in CREMOPHOR EL®:ethanol (70% and 30% w/w respectively) and further diluted 1:20 in physiological buffer, generally saline, to reach the appropriate dose. Thus, the vehicle is the original “solvent” diluted in the appropriate buffer.

All experimentations in animals were performed under humane conditions according to the Israeli Law for Animal Protection—Experiments in Animal 1994. All studies were reviewed by internal ethics committee and approved by the National responsible authority. Unless otherwise stated, animals were acclimated one week before initiation of study, and maintained under controlled environment. Animals were housed, at most 5 per cage for rats and at most 10 per cage for mice, on a 12 hours light/12 hours dark regimen, at a constant temperature of 22±4° C. and controlled humidity of 55±15% RH, with pellets of rodent diet and drinking filtered water ad libitum. At the end of the experiments, the animals were euthanized with an i.p. injection of 100 mg/kg sodium pentobarbitone (CTS). As a rule, the experiments were performed and the various scores measured by persons blinded to the treatment group.

### Biological Section

### Example 13

### Binding Affinity for the CB₁ and CB₂ Receptors

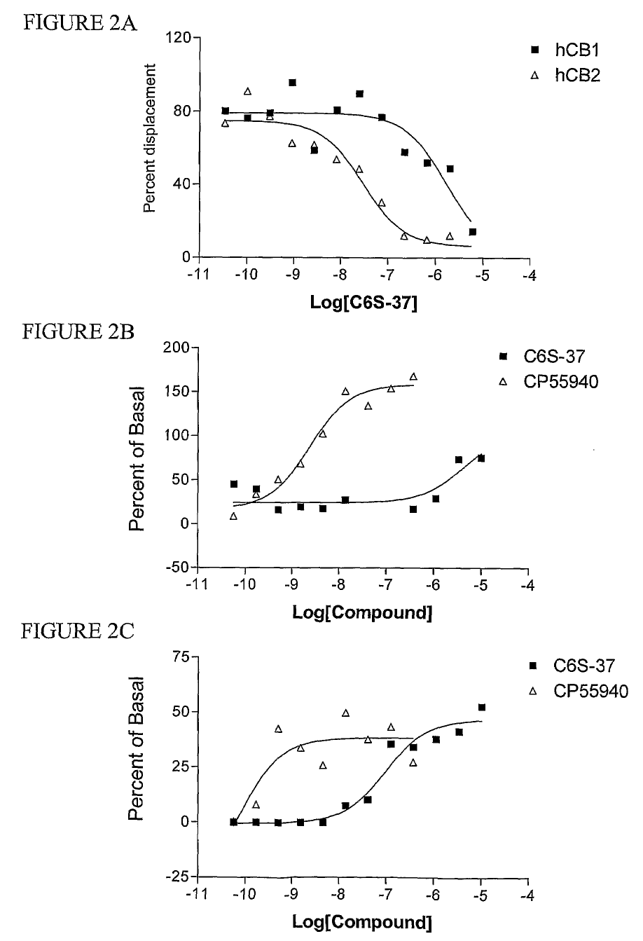

The binding assays were performed by testing the ability of the new compounds to displace the radiolabeled synthetic non-selective cannabinoid agonist [³H]CP55940 (168 Ci/mmol; PerkinElmer) from the human CB₁ (hCB₁) or human CB₂ (hCB₂) receptor on membranes derived from stably transfected HEK-293 cells (PerkinElmer). Membranes were diluted in assay buffer (50 mM Tris-HCl, 2.5 mM EDTA, 5 mM MgCl₂, 0.5 mg/ml BSA, pH=7.4). The amount of membrane was determined for each batch of membranes according to protein binding assay. The minimum amount of membrane that gave 50% specific binding was used for the binding assay. In most assays, binding was tested using 8 μg and 4 μg protein of hCB₁ and hCB₂ membranes, respectively. The tested compounds were dissolved in DMSO and diluted in assay buffer to a final concentration of 2.5% solvent. Total binding of [³H]CP55940 was evaluated with 1.5 nM to hCB₁ and with 1 nM to hCB₂, according to K_{d} affinity of [³H]CP55940 for the respective membranes. The ability of the tested compounds to displace [³H]CP55940 was evaluated first at single concentration points of either 10, 100, 300, 500 or 1000 DM for binding toward hCB₁ or hCB₂. In certain cases, the displacement was tested at compound concentrations ranging from 0.03 nM to 6 μM. Non-specific binding was measured by the addition of 6 μM of unlabelled CP55940 to the tubes. Binding assays were performed in triplicate in a total volume of 200 μl for 60 minutes at 30° C., in a shaking bath. Free and bound radioligands were separated by rapid filtration through 96-well GF/C harvesting filter plates (PerkinElmer) that had been presoaked with 0.1% Polyethylenimine (Sigma). Filters were dried and incubated for 30 minutes with 0.025 ml scintillation fluid (PerkinElmer) and radioactivity was determined by liquid scintillation counter (Topcount; PerkinElmer). For binding analysis, log concentration was plotted versus percent of specific binding out of total binding (Prism; GraphPad). IC₅₀ values were extrapolated from this plot and Ki values were calculated from the specific concentration of [³H]CP55940 that was added in each assay. FIG. 2 Panel A shows such a plot for exemplary compound C6S-37.

Results are reported in FIG. 1. For compounds tested over a range of concentrations allowing the appropriate calculations, the value reported represents the Kᵢ of the compound in nM. For compounds tested at single concentrations of either 10, 100, 300, 500 or 1000 nM for hCB₁ or hCB₂ binding, the value reported represents the percentage of binding displacement achieved by the tested compound at said concentration. Compounds tested at single concentrations were initially assayed at 500 nM for hCB₁ affinity and at 100 nM for hCB₂ affinity, percent inhibition at these concentrations is reported in the last column of FIG. 1. One asterisk in said column indicates that the compound was tested at 500 nM for both hCB₁ and hCB₂, two asterisks indicate that the compound was tested at 500 nM for hCB₁ and at 1000 nM for hCB₂ and three asterisks indicate that the compound was tested at 1000 nM for both hCB₁ and hCB₂ A high percentage indicates a compound with higher affinity toward the specific receptor being studied.

As can be seen in FIG. 1, compounds of the invention either bind or not to human cannabinoid receptors at the concentrations tested. Certain compounds bind more selectively one CB receptor over the other, whereas other compounds have relatively comparable affinities toward both receptors.

### Example 14

### [³⁵S]GTPγS-Binding Assay

Functional activity of compounds of the invention toward the cannabinoid receptors was determined by stimulation of [³⁵S]-GTPγS binding using membranes from HEK-293 cells expressing the hCB₁ receptor and membranes expressing the hCB₂ receptor derived from either Sf9 (PerkinElmer) or from HEK-293 cells. Activities were compared to that of the known cannabinoid full agonist CP55940 (Alexis). The purpose of this experiment is to determine the potency of the compounds of the invention as agonists or antagonists toward each of the receptor tested.

[³⁵S]-GTPγS binding reactions were performed at 30° C. in 96-well plates containing 5-10 μg membrane protein suspended in 0.1 ml binding buffer [20 mM HEPES-NaOH, pH 7.4, 5 mM MgCl₂, 100 mM NaCl, 0.2% (w/v) bovine serum albumin] supplemented with 50 μM GDP and 0.06 nM-10 μM of the compound being tested. Binding was initiated by the addition of [³⁵S]GTPγS (0.3 nM final concentration). Incubations were performed for 90 minutes and were terminated by filtration on GF/C filter plates (PerkinEimer). Filters were washed ten times with ice-cold wash buffer (20 mM HEPES-NaOH, pH 7.4, 10 mM sodium pyrophosphate). Non-specific binding was measured in the presence of 15 μM GTPγS.

Assays were performed in duplicates. Data was analyzed by plotting on the X axis the log concentration against percent of specific [³⁵S]GTPγS binding out of basal [³⁵S]GTPγS binding on the Y axis, non-linear regression is then performed using GraphPad Prism, version 3.0 (GraphPad, San Diego, Calif.) to calculate the EC₅₀ and Eₘₐₓ of the compound. The EC₅₀ value represents the concentration at which there is 50% [³⁵S]GTPγS binding and the Eₘₐₓ value the upper plateau of the curve.

FIG. 2 Panels B and C show such plots when exemplary compound C6S-37 was assayed for functional activity toward hCB₁ and hCB₂, respectively. The EC₅₀ and Eₘₐₓ values of selected compounds are presented in Table 1 below.

TABLE 1
GTPγS.

| | Human CB₁ Receptor | | Human CB₂ Receptor | |
|---|---|---|---|---|
| Compound | EC₅₀ (nM) | Eₘₐₓ (%) | EC₅₀ (nM) | Eₘₐₓ (%) |
| C6S-17 | 2878 | −25 | 4 | 43 |
| C6S-27 | NB | NB | 5562 | 64 |
| C6S-37 | 3974 | 102 | 24 | 44 |
| C6S-38 | NB | NB | 184 | 19 |
| C6M-8 | 0.25 | −25 | NB | NB |
| C6M-10 | 5300 | 132 | 25 | 72 |
| C7S-2 | 359 | 50 | 227 | 26 |
| C7S-9 | NB | NB | 712 | 52 |
| C7S-26 | 20 | 155 | 0.07 | 38 |
| C7S-28 | 217 | 118 | 16 | 44 |
| C7S-29 | 79 | 41 | 54 | 60 |
| C7S-30 | NB | NB | 1026 | 66 |
| C7S-31 | NB | NB | 641 | 54 |

For comparison, the full agonistic activity elicited with control cannabinoid CP55940 yielded Eₘₐₓ values of 50 to 100% at the CB₁ receptor and of 30 to 60% at the CB₂ receptor. In each experiment, the EC₅₀ values of the control were comparable to what has been reported in the literature. Compounds having an EC₅₀ value below 100 nM are considered to be potent agonists. The results shown in the above table demonstrate that some compounds of the invention have agonistic activity toward cannabinoid receptors, which is either selective or not. For instance, compounds C6S-17, C6S-37 and C6M-10 are agonists specific toward the CB₂ receptor, whereas C7S-26, C7S-28 and C7S-29 are agonists toward both receptors with some degree of selectivity toward the CB₂ receptor. While C7S-29 can be considered a non-selective agonist, C7S-28 is about 10-fold selective toward CB₂ and C7S-26 is about 285-fold selective. Cannabinoid agonists and antagonists have recognized therapeutic benefit.

### Example 15

### Anti-Inflammatory Effect in Activated Macrophages

This study was designed to assess in vitro the anti-inflammatory and immunomodulatory activity of compounds of the invention. The anti-inflammatory activity is assessed in immune cells activated to transcribe and secrete inflammatory mediators. This activity is measured at two levels, first at the level of gene transcription and at the level of protein secretion. The inducer used in the present study, Lipopolysaccharide (LPS), is known to be critical for the innate immune response to gram-negative bacteria in numerous pathological conditions.

RAW 264.7 macrophages, a mouse cell line (ATCC # TIB 71), were grown in Dulbecco's modified Eagle's medium (DMEM) with 4 mM L-glutamine adjusted to contain 1.5 g/L sodium bicarbonate, 4.5 g/L glucose, and 10% heat inactivated fetal bovine serum. Cells were grown in tissue culture flasks and seeded at appropriate density into 6 wells tissue culture plates. Four million Raw cells in half a milliliter were stimulated with 1 μg/ml LPS E. coli 055:B5 (DIFCO Laboratories). The mouse macrophages were pre-treated for one hour with controls or 10 μM of test compounds, and later on activated with LPS. RNA samples were extracted from the cells 3 hrs after activation and gene expression levels were analyzed by real-time RT-PCR. In parallel, supernatants were collected and secretion of inflammatory mediators was analyzed using ELISA techniques according to the instructions of the kit manufacturer.

Total RNA is prepared using SV total RNA isolation system (Promega). The cells were homogenized in lysis buffer. The lysates were transferred to an RNA isolation column, treated with DNAse, washed and eluted according to kit instructions. RNA concentrations were determined using GeneQuant II (Pharmacia-Amersham). Complementary DNA (cDNA) was synthesized from total RNA using SUPERSCRIPT II reverse transcriptase (Life Technologies). 2 μg of total RNA were combined with an oligo (dT)₁₅ primer, 0.5 mM dNTP mix, 8 units of reverse transcriptase and other reaction components up to a final volume of 20 μl, according to the kit instructions. The reaction mixture was incubated at 42° C. for 45 min and inactivated at 70° C. for 15 minutes. Quantitative real-time RT-PCR included 1 μl of the cDNA, 300 nM of the appropriate forward and reverse primers (according to the gene monitored) and 7.5 μl of the reaction mix containing buffer, nucleotides, Taq polymerase and SYBER green (SYBER Green master mix, Applied Biosystems), in a total reaction volume of 15 μl. Gene amplification was obtained using the GeneAmp 5700 sequence detection system (Applied Biosystems). Amplification included one stage of 10 minutes at 95° C. followed by 40 cycles of a 2-steps loop: 20 seconds at 95° C., and 1 minute at 60° C. During each annealing step, the amount of the amplified product was measured by the fluorescence of the double strand DNA binding dye, SYBER Green. The cycle of threshold (C_{T}), representing the PCR cycle at which an increase in fluorescence above a baseline signal can be first detected, was determined for each product. A delay of one PCR cycle in the C_{T} is translated into a two-fold decrease in starting template molecules and vice versa. The changes in the C_{T} of the specific gene product were normalized to the changes in the C_{T} of housekeeping cyclophilin or GAPDH as reference genes. Results were expressed as fold increase of gene expression in treated or untreated activated cells above the resting cells, after normalization to cyclophilin or GAPDH. Cells were also tested for viability to confirm that any effect on gene expression was indeed due to modulation of transcription of a specific target and not to cytoxicity. The compounds of the invention were found to be safe to the cells at the dose tested.

In the following list, the letters f and r indicate the forward and reverse primers, respectively.

Primer sequences used:

| | |
|---|---|
| Mouse COX-2 f | |
| 5′-TTCCGTTTCTCGTGGTCACTT-3′ | (SEQ ID NO: 1) |
| Mouse COX-2 r | |
| 5′-AGCGCTGAGGTTTTCCTGAA-3′ | (SEQ ID NO: 2) |
| Mouse IL-1β f | |
| 5′-ACACTCCTTAGTCCTCGGCCA-3′ | (SEQ ID NO: 3) |
| Mouse IL-1β r | |
| 5′-CCATCAGAGGCAAGGAGGAA-3′ | (SEQ ID NO: 4) |
| Mouse IL-10 f | |
| 5′-GCCCTTTGCTATGGTGTCCTT-3′ | (SEQ ID NO: 5) |
| Mouse IL-10 r | |
| 5′-TCCCTGGTTTCTCTTCCCAA-3′ | (SEQ ID NO: 6) |
| Mouse iNOS f | |
| 5′-TTCCAGGTGCACACAGGCTA-3′ | (SEQ ID NO: 7) |
| Mouse iNOS r | |
| 5′-GCACGCTGAGTACCTCATTGG-3′ | (SEQ ID NO: 8) |
| Mouse MCP-1 f | |
| 5′-TCACAGTTGCCGGCTGG-3′ | (SEQ ID NO: 9) |
| Mouse MCP-1 r | |
| 5′-TCTTTGGGACACCTGCTGCT-3′ | (SEQ ID NO: 10) |
| Mouse TNF-α f | |
| 5′-AAGGACTCAAATGGGCTTTCC-3′ | (SEQ ID NO: 11) |
| Mouse TNF-α r | |
| 5′-CCTCATTCTGAGACAGAGGCAAC-3′ | (SEQ ID NO: 12) |
| Mouse cyclophilin A f | |
| 5′-TCGCCATTGCCAAGGAGTAG-3′ | (SEQ ID NO: 13) |
| Mouse cyclophilin A r | |
| 5′-GGTCACCCCATCAGATGGAA-3′ | (SEQ ID NO: 14) |
| Mouse GAPDH f | |
| 5′-GGTTGTCTCCTGCGACTTCAA-3′ | (SEQ ID NO: 15) |
| Mouse GAPDH r | |
| 5′-GTAGGCCATGAGGTCCACCA-3′ | (SEQ ID NO: 16) |

The expression of genes encoding inflammatory mediators was significantly increased following LPS activation of RAW cells. LPS activated cells displayed 1189-, 86-, 202-, 261-, 760- and 160-fold overexpression, over resting cells, for COX-2, IL-1β, IL-10, iNOS, MCP-1 and TNF-α, respectively. Results were further expressed as percent inhibition of gene expression in compound treated activated cells over vehicle “treated” activated cells. Since the modulation of IL-10 and TNF-α gene expression by the compounds of the invention is relatively minor, it is not reported. Results for COX-2, IL-1β, iNOS, and MCP-1 are presented in Table 2 below. NO indicates that the compound tested did not significantly affect the transcription of the gene assessed and that its activity is comparable to vehicle, within ±20% from this control. Inhibition above 50% was considered significant.

TABLE 2
Percent inhibition of gene expression.

| Compound | COX-2 | IL-1β | iNOS | MCP-1 |
|---|---|---|---|---|
| C5S-1 | 29% | 47% | 37% | NO |
| C5S-2 | NO | 81% | NO | NO |
| C6S-1 | NA | NO | NO | 66% |
| C6S-2 | 61% | 58% | 23% | 44% |
| C6S-3 | 65% | 74% | 68% | 55% |
| C6S-6 | NO | 37% | NO | NO |
| C7S-2 | 26% | NO | NO | NO |
| C6M-8 | NA | 51% | 66% | 85% |
| C6M-9 | 50% | NO | 32% | 37% |

The results shown in the above table demonstrate that compounds of the invention have anti-inflammatory and immunomodulatory properties as expressed by their ability to decrease the expression of genes involved in inflammatory and immune processes. Certain compounds inhibited the expression of all genes tested, for instance compound C6S-3, which displayed significant inhibiting activity. On the other hand, certain compounds displayed at the dose tested more specific inhibition. For example, C5S-2 inhibited selectively IL-1β, expression by 81%, C6S-1 inhibited MCP-1 expression by 66%, C7S-2 inhibited COX-2 expression by 26% and C6S-7 inhibited IL-10 expression by 33%.

### Example 16

### Anti-Inflammatory Effect in LPS Injected Mice

This study was designed to assess in vivo the anti-inflammatory activity of compounds of the invention. The anti-inflammatory activity is assessed in mice systemically exposed to LPS to induce the secretion of inflammatory mediators into blood circulation.

Balb/C female mice (average body weight 20 g, Harlan, Israel) were injected i.v. at a volume dosage of 5 ml/kg with either vehicle or test compounds at a dose of 2 mg/kg. Each treatment group comprised at least 9 animals. Immediately after compound or control administration, the mice were injected i.p. with 3 mg/kg LPS (E. coli 055:B5, Calbiochem). Ninety minutes after LPS induction, blood samples were collected into heparanized test tubes. Plasma was separated by centrifugation (10,000 rpm for 5 minutes at RT) and stored at −20° C. until assayed. The level of the inflammatory mediator under study, IL-1β, IL-6, IL-10 or TNF-α, was assayed by ELISA techniques.

The technique used to quantify the amount of a given protein in a liquid sample, either tissue culture supernatant or body fluid, is based on Enzyme Linked ImmunoSorbent Assay (ELISA) methodology. Either commercially available or established in house, the assay is based on the capture of the protein of interest by specific antibodies bound to the bottom of an ELISA plate well. Unbound material is washed away, the captured protein is then exposed to a secondary antibody generally labeled with horseradish peroxidase (HRP) or alkaline phosphatase (ALP). Again the unbound material is washed away, and the samples are then incubated with the appropriate substrate yielding a colorimetric reaction. The reaction is stopped and reading is performed in a spectrophotometer at the appropriate wavelength. Samples are tested at least in duplicate and the appropriate standard curve, consisting of serial dilutions of the recombinant target protein, is incorporated on each plate. The concentration of the protein in the sample is calculated from the standard curve.

The results are expressed as percent inhibition of secretion, taking into account the maximal cytokine concentration in vehicle “treated” animals and the baseline level in naïve animals. The level of inhibition of cytokine secretion obtained in this study by compounds of the invention, are reported in Table 3 below. NO indicates that the compound tested did not significantly affect the level of cytokine in plasma of LPS injected mice and that its activity is comparable to vehicle, within ±20% from this control. Inhibition above 50% was considered highly significant.

TABLE 3
Inhibition of cytokine secretion into blood circulation.

| Compound | IL-1β | IL-6 | IL-10 | TNF-α |
|---|---|---|---|---|
| C5S-1 | 41% | 28% | 75% | 77% |
| C5S-2 | 53% | NO | 64% | 38% |
| C6S-2 | 56% | NO | 68% | NO |
| C6S-3 | NO | NO | 79% | 78% |
| C6S-5 | NO | 85% | 84% | NO |
| C7S-1 | NO | 82% | 82% | NO |

The results shown in the above table demonstrate that compounds of the invention have anti-inflammatory properties in vivo as expressed by their ability to decrease the level of inflammatory mediators in plasma of animals subjected to systemic LPS exposure. This activity has a wide range of therapeutic applications.

### Example 17

### Analgesic Effect on Visceral Pain

In the present study, the analgesic activity of compounds of the invention was assessed in a model of visceral pain. Visceral pain is caused by disorders of internal organs such as the stomach, kidney, gallbladder, urinary bladder, intestines and others. Visceral pain is nociceptive in nature and believed to be mediated by peritoneal resident cells, such as mast cells and macrophages. Visceral pain usually responds to opioids and NSAIDS. In the present study, the visceral pain was induced in mice by i.p. injection of acetic acid.

Male ICR mice (average body weight 25 g, Harlan, Israel) were pretreated by i.v. injection at volume dose of 5 ml/kg of vehicle, control and test compounds at various doses. Other routes of administration were tested for selected compounds including i.m. or s.c. injections at volume dose of 2.5 ml/kg and p.o. gavage at volume dose of 5 ml/kg. Compounds were dissolved in CREMOPHOR®:Ethanol and diluted 1:20 in saline prior to injection or gavage. Compounds administered i.v. were injected fifteen minutes before pain induction, whereas compounds administered i.m., s.c. or p.o. were supplied thirty minutes before pain induction, unless otherwise indicated. Each treatment group, except for controls comprising at least 30 animals, was composed of at least 6 animals. Fifteen or thirty minutes after drug administration, depending on the route of administration, the mice were injected i.p. with 10 ml/kg of 0.6% acetic acid and the number of visceral pain related behaviors (writhing movements globally defined as WR, i.e. stretching, contractions of the abdomen accompanied by an elongation of the body and extension of the hind limbs) was counted over a period of 5 minutes, starting 5 minutes after the acetic acid administration. These visceral pain related behaviors were globally defined as writhing responses (WR). The results are expressed as mean number of writhing responses ±SEM. Data were analyzed using analysis of variance (ANOVA) followed by post-hoc Fisher test. A value of p<0.05 was considered to be statistically significant, generally when compounds inhibited between 25 to 50% of the writhing responses, and is indicated on the figure by an asterisk over the relevant treatment group. A value of p<0.01, indicated on the figure by two asterisks, was considered highly significant and was generally observed when compounds inhibited more than 50% of the writhing responses.

In the first part of the study, the compounds of the invention were tested at a single dose of 2 mg/kg i.v. Results, expressed as percent inhibition of writhing responses as compared to untreated group, are shown in Table 4 below. Untreated animals displayed on average 28.3±2.5 writhing responses and vehicle only has no effect, with an observed number of writhing responses of 26.0±1.6.

TABLE 4
Percent Inhibition of Visceral Pain Writhing Responses.

| | Compound | | | | | |
|---|---|---|---|---|---|---|
| | C5S-1 | C5S-2 | C5S-4 | C5S-5 | C6S-1 | C6S-5 |
| % Inhibition | 41% | 72% | 100% | 27% | 39% | 35% |
| | Compound | | | | | |
| | C6S-6 | C6S-7 | C6S-8 | C6S-10 | C6S-17 | C6S-39 |
| % Inhibition | 38% | 40% | 37% | 28% | 38% | 100% |
| | Compound | | | | | |
| | C7S-1 | C7S-2 | C7S-6 | C7S-9 | C7S-10 | C7S-13 |
| % Inhibition | 76% | 100% | 99% | 26% | 53% | 72% |

| | Compound | | | | |
|---|---|---|---|---|---|
| | C7S-26 | C7S-28 | C7S-29 | C7S-30 | C6M-9 |
| % Inhibition | 26% | 56% | 100% | 92% | 38% |

The results shown in the above table demonstrate that compounds of the invention have potent analgesic activity. Six of the compounds tested, C5S-4, C6S-39, C7S-2, C7S-6, C7S-29 and C7S-30, inhibited by more than 90% the writhing responses in the treated animals, at the relatively low dose of 2 mg/kg i.v. The analgesic activity being dose related, as shown below, other compounds needed higher doses between 4 to 10 mg/kg to yield similar abrogation of the pain response. For comparison, the NSAID celecoxib at doses up to 10 mg/kg was inactive in this model, whereas the opiate morphine eradicated the pain response at 2 mg/kg i.v.

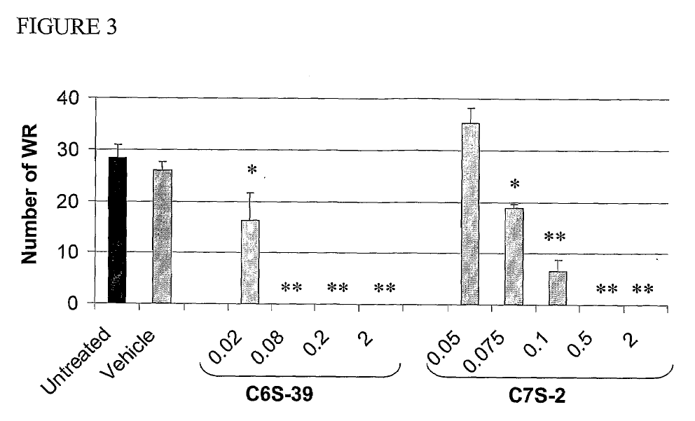

In a separate study, it was shown that the analgesic activity of selected compounds of the invention is dose related. C6S-39 and C7S-2 which totally inhibited the pain response at 2 mg/kg i.v. were selected for testing over a range of doses starting respectively at 0.02 and 0.05 mg/kg. Results, expressed as the number of writhing responses, are shown in FIG. 3. C6S-39 and C7S-2 were found to be already potent at the low doses of 0.02 and 0.075 mg/kg respectively, where they inhibited the writhing responses by 38% and 28% as compared to vehicle. At the dose, of 0.08 mg/kg, C6S-39 totally inhibited pain response, indicating that its estimated IC₅₀ is of only 0.03 mg/kg. At 0.1 mg/kg C7S-2 inhibited 74% of the writhing responses and it totally eradicated pain responses already at 0.5 mg/kg. The calculated IC₅₀ for compound C7S-2 in visceral pain is of only 0.09 mg/kg. For comparison, in this model morphine yielded an IC₅₀ of 1.07 mg/kg, showing that C6S-39 and C7S-2 are indeed very active analgesic agents 35- and 12-fold more potent than morphine.

In another study, the impact of the route of administration was evaluated. As described above the IC₅₀ of compound C7S-2 when administered i.v. was found to be of only 0.09 mg/kg. When this compound was administered i.m., it was found to be potent already at 0.25 mg/kg with 35% inhibition of writhing responses. At 0.5 mg/kg the percent inhibition raised to 90% and at 1 mg/kg i.m., the compound totally abrogated the pain response. The calculated IC₅₀ following i.m. administration is of about 0.27 mg/kg. When C7S-2 was administered p.o. it was already very potent (58% inhibition) at the lowest tested dose of 10 mg/kg and inhibited 83% of the writhing responses at 20 mg/kg. The calculated IC₅₀ following p.o. administration is of about 6.6 mg/kg. Finally, this compound was administered subcutaneously and it was found to have an estimated IC₅₀ of about 1 mg/kg by this route of administration. This study shows that in all routes of administration tested this exemplary compound retained highly potent analgesic activity.

In another study, this experimental setup was used to assess whether salt derivatives would maintain the activity of the parent compounds. For this purpose C7S-32 was compared to its HCl salt, and both compounds were tested at 4 and 10 mg/kg i.v. It should be noted that the dosage being as weight compound per animal body weight, the animals administered the salt derivative received in fact only 90% of the parent compound. Parent C7S-32 inhibited 38 and 100% of pain response at 4 and 10 mg/kg respectively, while its salt inhibited 50 and 84% of the writhing response. These highly similar analgesic activities support the potency of salt derivatives.

Taken together, these results support the potent analgesic activity of compounds of the invention via numerous routes of administration.

### Example 18

### Analgesic Effect on Inflammatory Pain

The purpose of this study is to test the anti-inflammatory pain activity of the compounds. Inflammatory pain is nociceptive in nature, wherein the pain sensation is often perceived for longer period than in acute pain such as elicited in Example 17. Wherein in visceral pain, the prophylactic analgesic activity of the compounds was assessed for up to about half-hour, in the present model the duration of the preventive activity of compounds against acute pain was assessed for up to about three hours. Inflammatory pain and paw edema were induced by injection of 2% λ carrageenan in the animal hind paw.

Male Sprague Dawley rats (average body weight 200 g, Harlan, Israel) were transiently sedated by placement on dry ice for the duration of the injections. Rats were injected subcutaneously, in the subplantar region of one (right) paw with 0.1 ml of 2% w/v λ Carrageenan in sterile saline. The contralateral (left) paw was not injected as data from the literature, confirmed by our own experience, showed that injection of 0.1 ml of normal saline did not affect later analgesic measurements. Test compounds were, unless otherwise stated, administered i.p. at initial single dose of 3, 10 or 20 mg/kg, and volume dose of 5 ml/kg, immediately after the carrageenan injection. Selected compounds were also tested p.o. following oral gavage. Vehicle and celecoxib treated animals were used as controls. Each treatment group comprised at least seven animals.

Before induction of inflammatory pain and three hours after injection, the animals reactions to pain stimuli were tested in two systems. The first stimulus was thermal and assessed by the Plantar Test according to Hargreaves, using Ugo Basile Model 7370. The scale was set to an intensity of 50 arbitrary units. The latency time till the animal lift a paw as a reaction to the thermal stimulus was recorded for both the inflamed and non-inflamed hind paws. The second stimulus was mechanical (tactile) and assessed using a Dynamic Plantar Sesthesiomether (Ugo Basile Model 73400-002). The system was set on maximal force of 50 grams and the force applied was gradually increased at the rate of 10 g/sec. Finally, the impact on paw edema was assessed. Paw thickness was measured using a dial thickness gauge (Spring-dial, constant low pressure gauge, Mitutoyo, TG/L-1, 0.01 mm) and paw volume was measured using a plethysmometer (model #7150, Ugo Basile, Italy). At the end of the study, animals were euthanized.

The results are measured as the differences between the two hind paws at time 0 and 3 hours both as ALT, for the latency time in the thermal part of the study, and as ΔForce, for the mechanical part of the study. The paw volume is expressed as percent from vehicle treated animals. Results are expressed as mean±SEM for each treatment group and the differences among those groups are analyzed by analysis of variance (ANOVA) followed by post-hoc Tukey's test.

Administration of 2% λ carrageenan induced localized paw inflammation, characterized by swelling and redness of the paws. The paws of vehicle treated animals almost doubled in volume as compared to naïve paws (96% swelling over baseline). Before inflammatory pain induction by carrageenan injection, the difference in latency time between the hind paws following thermal stimuli is of about 0.9 second. Three hours later, vehicle treated animals displayed a ΔLT of 9.7 seconds between the normal and injured paw. Similarly, the baseline values for the difference in force to be applied between the hind paws following mechanical stimuli is of about 0.5 gram before pain induction, whereas three hours later, vehicle treated animals displayed a ΔForce of 26 grams between the normal and injured paw.

Compounds of the invention reduced these outcomes and the results presented in the following table are expressed as percent reduction in paw swelling, ΔLT and Δforce according to the parameter measured. Results refer to i.p. treatment with 10 mg/kg. Results marked with one asterisk refer to a dose 3 mg/kg, whereas two asterisks refer to 20 mg/kg. Celecoxib is included as reference.

TABLE 5
Percent Inhibition of Inflammatory Pain Responses.

| Compound | Edema | ΔLT | ΔForce |
|---|---|---|---|
| Celecoxib | 45% | 58% | 2% |
| C5S-2 | 48% | 43% | 8% |
| C5S-4 | 49% | 100% | 88% |
| C6S-1** | 48% | NA | NA |
| C6S-2 | 36% | 58% | 30% |
| C6S-3 | 48% | 84% | 49% |
| C6S-5 | 56% | 61% | 34% |
| C6S-7 | NA | 54% | 41% |
| C6S-9 | NA | 53% | 14% |
| C6S-10** | 37% | 50% | 24% |
| C6S-12 | 53% | 33% | 37% |
| C6S-16** | 31% | 69% | 6% |
| C6S-17* | 44% | 60% | 37% |
| C6S-18** | 48% | 49% | 31% |
| C6S-21** | 38% | 44% | NA |
| C7S-1 | 60% | 0% | 13% |
| C7S-2* | 56% | 66% | 66% |
| C7S-3 | 43% | 46% | 35% |
| C7S-10 | 41% | 61% | 25% |
| C7S-13 | 34% | 0% | 6% |
| C6M-1 | 31% | 12% | 12% |
| C6M-7 | 26% | NA | NA |
| C6M-8 | NA | 45% | 20% |
| C6M-9 | 49% | 75% | 27% |

The results shown in the above table confirm that compounds of the invention have anti-inflammatory and analgesic activity in vivo and demonstrate that they are at least as potent as the known NSAID celecoxib. Some of the compounds seem more active against a specific aspect measured in this model, whereas others are highly potent at all three parameters. It should be noted that celecoxib was not effective in mechanical hyperalgesia, supporting the analgesic advantage of certain compounds of the invention over this commercially available drug. For example, compounds C5S-4, C6S-2, C6S-3, C6S-5, C6S-12, C6S-17, C7S-2, C7S-3 and C6M-9 are comparable or superior to celecoxib as far as reduction of edema and thermal hyperalgesia are concerned. However, they are clearly superior to this reference drug with inhibition of mechanical hyperalgesia ranging from 27% to 88%, as compared to the lack of effect (2% inhibition) of celecoxib. Compounds of the invention may also advantageously replace NSAIDs as far as side effects are concerned.

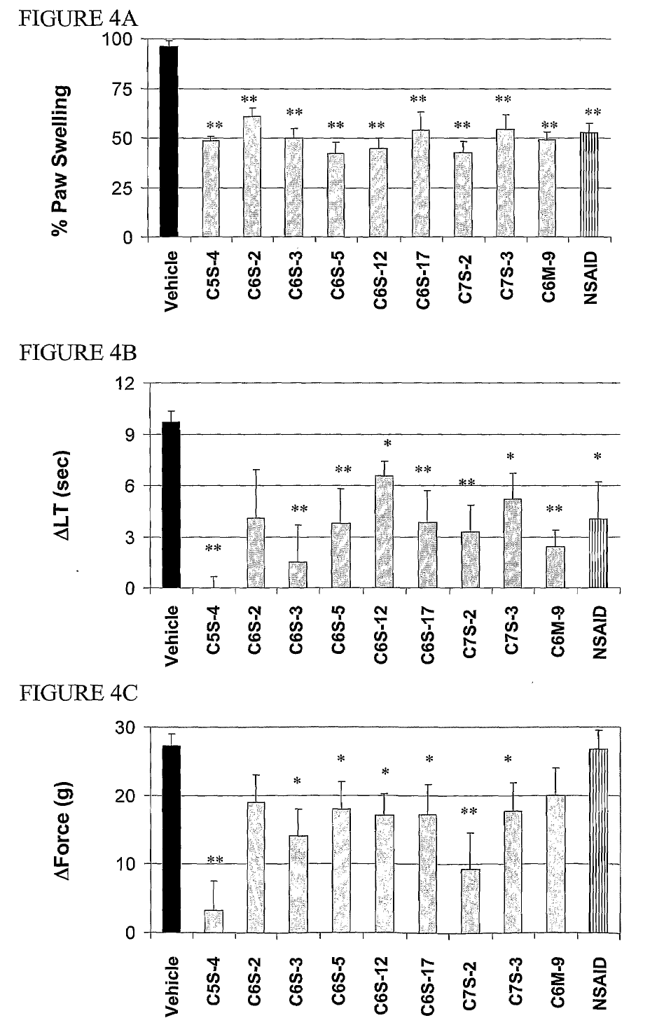

The results achieved by these compounds are shown in FIG. 4. Panel A depicts the paw swelling three hours after carrageenan injection and treatment, as percent over baseline. Panel B depicts the difference in Latency Time between the paws, in seconds, following thermal stimulus. Panel C depicts the difference in Force that will cause the animal to withdraw its injured vs. control paw, in grams, following mechanical stimulus. A statistically significant value of p<0.05 is indicated on the figure by an asterisk over the relevant treatment group, whereas two asterisks indicate a p value below 0.01.

In an additional study, selected compounds were tested for oral efficacy over a range of doses. C7S-2 was already significantly potent at 5 mg/kg p.o. reducing by 41% the paw swelling, as compared to 50% reduction at 20 mg/kg p.o. C7S-10 was highly effective at 30 mg/kg p.o. inhibiting 60% of paw swelling, with no better anti-inflammatory effect at higher doses. These results indicate that in this model and experimental set-up compounds could not achieve more than about 60% reduction in paw swelling. This specific study supports, as previously observed in the visceral pain model, that compounds of the invention are effective via various routes of administration.

### Example 19

### Analgesic Effect on Neuropathic Pain

Neuropathic pain, associated with chronic pain, differs from previously assessed visceral and inflammatory pain, associated with acute pain. Acute pain and chronic pain differ in their etiology, pathophysiology, diagnosis and treatment. Acute pain is nociceptive in nature and occurs secondary to chemical, mechanical and thermal stimulation of A-delta and C-polymodal pain receptors. Acute pain is self-limiting and will vanish on short-term after initial injury. Chronic pain, on the other hand, is continuous and can persist for years after the initial injury. It is produced by damage to, or pathological changes in the peripheral or central nervous system. Neuropathic pain tends to be only partially responsive to opioid therapy. Drugs active against certain types of acute pain such as visceral pain and inflammatory pain are therefore not necessarily effective against neuropathic pain.

The analgesic activity of compounds of the invention was assessed in a chronic constriction induced (CCI) model of neuropathic pain. A peripheral monopathy was induced in the right hind limb of rats following a chronic constriction of the sciatic nerve according to Bennet et al. [Bennet, G. J. & Xie, Y-K., Pain 33: 87-107, 1988]. The development of mechanical allodyna was monitored using a Dynamic Plantar Sesthesiomether as described in Example 18. This apparatus is an automated version of the classical von Frey filaments' test.

Pre-surgery baseline values were ascertained as the mean of 2 pre-surgery values. Once the baseline values are established, the animals were surgically prepared by constricting the right sciatic nerve with 4-0 chromic cat gut loose ligatures. On day 11 post-operation, the animals that have developed mechanical allodyna were arbitrarily allocated to the various treatment groups based on the pre-surgery values.

The design was randomized, performed in a masked fashion as to whether drug or vehicle is being given. Male Sprague Dawley rats (average body weight 240 g, Harlan, Israel), were allowed to acclimatize to the behavioral testing equipment prior to testing. On the testing day, the animals, at least six per treatment group, were administered the compounds and controls at a volume dosage of 5 ml/kg. Fifteen minutes later, the mechanical stimulus was applied and the force, measured in grams, causing a withdrawal response for each of the ipsilateral and contralateral hind paws was evaluated.

Results are expressed as mean±SEM for each treatment group and the differences among those groups are analyzed by analysis of variance (ANOVA) followed by post-hoc Tukey's test. A value of p<0.05 is considered to be statistically significant. Thereafter the difference in the force to be applied to the injured paw as compared to the normal paw was calculated. Δforce, expressed in grams, was measured at baseline and 1 and 4.5 hours after treatment.

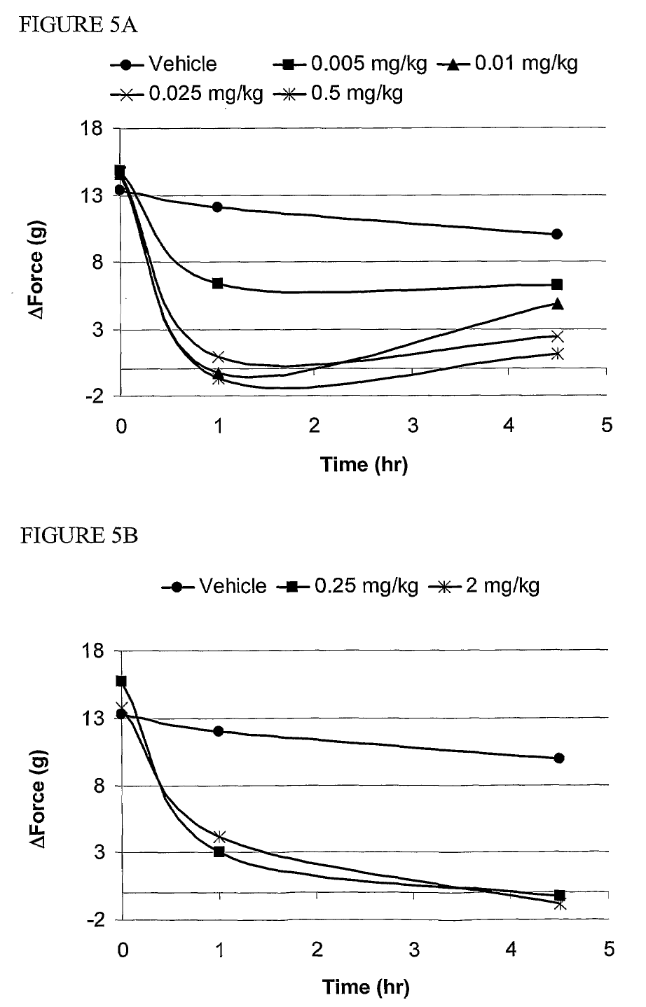

The results are presented in FIG. 5 where Δforce in grams is plotted for each treatment. Panel A relates to C7S-2 which was tested over the range of 0.005 to 0.5 mg/kg i.v. and Panel B relates to C7S-10 which was tested over the range of 0.25 to 2 mg/kg i.v.

The Δforce between the paws at baseline was of about 14.8 grams for all treatment groups. Animals administered vehicle only displayed a minor and non-significant reduction in Δforce over time. Four and a half hour after vehicle administration there was 25% reduction in Δforce as compared to baseline. Animals treated with doses as low as 0.005 mg/kg C7S-2 or 0.25 mg/kg C7S-10 displayed a significant decrease in Δforce over time, meaning a clear improvement of the injured paw. Four and a half hour after compound administration, animals treated with C7S-2 showed 58%, 67%, 84% and 92% inhibition of the pain behavior (expressed in Δforce) as compared to baseline for the doses of 0.005, 0.01, 0.025 and 0.5 mg/kg, respectively. At the last time point, both 0.25 and 2 mg/kg of C7S-10 totally abrogated the pain response, which they had already reduced by 70-80% one hour after compound administration.

These results indicate that compounds of the invention have a wide range of analgesic activities, which include the treatment of chronic pain, as induced in the present model.

### Example 20

### Effect on PLP Induced Remitting-Relapsing EAE

Experimental Autoimmune Encephalomyelitis (EAE), also called Experimental Allergic Encephalomyelitis, is an animal model of Multiple Sclerosis (MS). Various EAE models are known in the art, depending on the method of induction, the strain of the animal and the antigen employed to induce the disease. EAE is an acute or chronic-relapsing, acquired, inflammatory and demyelinating autoimmune disease. Different forms of EAE resemble very closely various forms and stages of MS in a large number of ways.

While Myelin Basic Protein (MBP) and Myelin Oligodendrocyte Glycoprotein (MOG) are used to induce the acute phase or the chronic progressive form of the disease, proteolipid protein (PLP) induces a remitting-relapsing type of disorder, which resembles more the initial pattern of neurodeficit outcome in MS patients.

SJL female mice (6 weeks old, Harlan, Israel) were administered s.c. in both flanks with 0.2 ml/mouse of emulsified Freund's adjuvant containing 125 μg of PLP and 300 μg of Mycobacterium Tuberculosis. Immediately after, the mice were administered i.p. with 0.3 ml/mouse of phosphate buffer saline (PBS) containing 600 ng of pertussis toxin. The same amount of the toxin was injected again 48 hours later. Animals were weighted and clinically evaluated daily and scored according to the following scoring system. O— no abnormality; 1—legs weakness; 2—limp tail; 3—limp tail and hind legs weakness; 4-partial hind legs paralysis; 5—hind legs paralysis and fore legs partial paralysis; 6—fore legs paralysis; and 7—moribund state.

Onset of disease was defined when animals could be clinically scored 1 or above (generally between day 7 to 10 from induction of disease). The first peak of disease was defined as an increase of at least one score unit sustained for at least two consecutive days after the animal has been injected with the disease inducing agents. Remission was achieved when animals demonstrated a reduction of at least 50% of the peak maximal score and stabilized to the new score for at least 2 days. Treatment was initiated at onset of disease and vehicle or compounds were administered daily for 10 days at volume dosage of 5 ml/kg. An additional control group was composed of untreated animals. Each treatment group comprised at least 8 mice. Animals were followed for up to two months and during this period two to three minor relapses were observed following the initial first peak of disease.

At the end of the study, mice were euthanized. Spinal cords, spleens and brains were removed and fixed in 4% formaldehyde solution prior to histological evaluation.

Results are expressed as mean±SEM and the differences between the treatment groups are analyzed by analysis of variance (ANOVA) followed by Tukey's post hoc test. A value of p<0.05 is considered to be statistically significant.

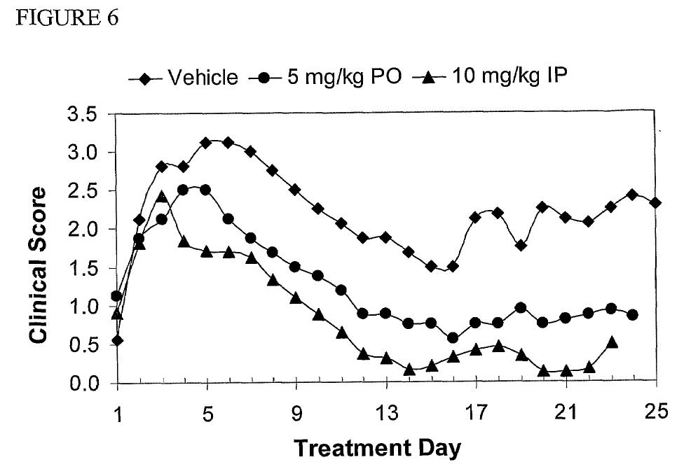

Results regarding the administration of 5 mg/kg p.o. and 10 mg/kg i.p. of compound C7S-2 are presented in FIG. 6 where the average clinical score is plotted against day since first treatment. Vehicle treated animals displayed a pattern similar to untreated animals (data not shown). As can be seen in FIG. 6, C7S-2 achieved significant effects, first it reduced the clinical score and shortened the duration of the first peak of the disease and second it almost totally prevented the occurrence of the relapses. The average clinical score on the first peak of the disease was of 3.63±0.25 for vehicle treated animals. This outcome was reduced to 2.78±0.15 for animals treated with C7S-2 at 5 mg/kg p.o. and to 2.43±0.26 for animals treated with 10 mg/kg i.p. This trend (respectively 23% and 33% reduction in average clinical score at peak as compared to vehicle) is significantly strengthened at the second peak of the disease (i.e. first relapse) where vehicle treated animals still display a very high average clinical score of 3.14±0.48, whereas C7S-2 p.o. reduces this outcome down to 1.25±0.41 and C7S-2 i.p. even further down to 0.57±0.11. When expressed in percent reduction as compared to vehicle, the values of 60% and 82% reduction in average clinical score for p.o. and i.p. administration respectively make it clear that the immunomodulatory effect is highly potent.

Over 23 days of treatment, the AUC for the animals treated with vehicle was of 54.13±9.58, whereas it was of only 28.13±5.74 for animals treated with C7S-2 at 5 mg/kg p.o. and of only 19.43±2.51 for animals treated with 10 mg/kg i.p. Thus over the period of the study, C7S-2 reduced the overall severity of the disease (expressed in AUC) by 48% when administered p.o. and by 64% when administered i.p.

These results demonstrate that compounds of the invention have not only anti-inflammatory effects in relatively acute models, but also strong immunomodulatory potential in chronic autoimmune diseases as exemplified in this model of multiple sclerosis. These results might also indicate potent neuroprotective activity since neural degeneration, axonal loss, neuroinflammation and nerve demyelination are hallmarks of this disease.

### Example 21

### Effect on CFA Induced Rheumatoid Arthritis

The purpose of this study is to evaluate the anti-inflammatory and analgesic activity of compounds of the invention in a model of chronic pain resulting from joint deformation initiated by inflammation, wherein Complete Freund's Adjuvant (CFA) is used to induce a situation similar to rheumatoid arthritis in humans.

Female Lewis rats (125 g average body weight, Harlan, Israel), at least eight per treatment group were used in this study. CFA was prepared by combining 100 mg of Mycobacterium Tuberculosis (Difco) with 5 ml of incomplete Freund's adjuvant and grinding for about 3 minutes the resulting mixture until a brownish suspension was obtained. The CFA suspension was administered s.c. at the base of the tail, 0.2 ml/animal. Three tests were performed to evaluate the pain and inflammation caused by the disease. These tests were performed before CFA injection to establish baseline values and 14, 21 and 28 days after disease induction. Compounds of the invention were administered daily for fourteen days p.o. at a dose of 10 mg/kg starting on day 14, after disease onset. A group of animals treated with vehicle only at 5 ml/kg served as control.

The parameters monitored were as detailed in Example 18 and include paw edema and redness, and response to thermal and mechanical pain stimuli. At the end of the treatment period the animals were euthanized. The paws were cut and stored in a solution of 4% formalin until histopathological evaluation.

The differences among various treatment groups between the severity of the clinical signs, as expressed by paw swelling and redness, as well as the differences in pain responses, as expressed by Latency Time (sec) and Force (g) needed to observe withdrawal of the paw following thermal and mechanical stimuli, and finally the tissue damage, as expressed by the histological scores, were compared using analysis of variance ANOVA followed by post-hoc t-Test. A value of p<0.05 is considered to be statistically significant.

The average clinical score regarding the edema was at initiation of treatment on day 14 similar for the vehicle treated animals, 6.00±1.18, and for the animals treated with 10 mg/kg p.o. of C7S-2, 5.86±1.70. After a week of treatment on day 21, the difference in average clinical score was of 1.89 unit in favor of C7S-2 treated animals. After two weeks of treatment, the clinical score for vehicle treated animals was highly similar to baseline with an average of 6.67±1.85, whereas C7S-2 treated animals displayed a significantly reduced score of only 3.29±0.97, representing a decrease of 44% in clinical score as compared to first day of treatment. Likewise when comparing the Latency Time or Force needed for an animal to lift a paw following thermal or mechanical stimulus at day 14 and 28, it was observed that vehicle treated animals displayed slight worsening at the end of the study with a Latency that was shortened by about 2 seconds following thermal stimulus and a Force that was lowered by 2.6 grams as mechanical stimulus needed to elicit a withdrawal response. On the other hand, animals that received 10 mg/kg p.o. C7S-2 were noticeably treated, as expressed by both parameters. The Latency was prolonged by about 5 seconds, representing more than 32% increase over baseline values of day 14, and the Force needed to elicit a withdrawal response was increased by 11.1 grams, which represent about 63% increase over baseline.

These results confirm that compounds of the invention have strong anti-inflammatory and immunomodulatory activities, which can be applied to a wide range of conditions.

### Example 22

### Effect on TNBS Induced Inflammatory Bowel Disease

The purpose of this study is to test the therapeutic activity of compounds of the invention in a model of inflammatory bowel disease (IBD). Various aspects of the disease can be studied depending on agent used for induction. For instance, oral administration of dextran sulfate sodium (DSS) cause an initial epithelial cell damage, followed by development of colitis and, eventually, by a relatively slow mucosal repair. The disease elicited in such a model is initially mediated by innate immunity, in particular by neutrophils. On the other hand, when the disease is induced by rectal administration of trinitrobenzesulfonic acid (TNBS), the initial disruption of the epithelial barrier leads to the activation of intestinal immune cells and the disease is mainly mediated by acquired immunity, in particular by T cells. This later model shares numerous features with human Crohn's disease, which is believed to result from dysregulated T helper 1 immune response.

Female Balb/C mice (average body weight 20 g, Harlan, Israel) were lightly sedated and challenged rectally, by instillation with 70 μl of 2.5% TNBS (Sigma) dissolved in 50% ethanol, to induce intestinal inflammation and colitis. Animals were weighted before the beginning of the study and treatment was administered i.p. on a daily basis for six days at 20 mg/kg and volume dosage of 5 ml/kg. During the study period of up to 7 days, the following parameters were daily monitored and recorded: body weight, presence of blood in the stool and stool consistency. These findings are scored according to Table 6 [Murthy S.N. et al., Dig. Dis. Sci. 38:1722-34, 1993].

TABLE 6
Criteria for Scoring Disease Activity Index (DAI^{#}) of IBD.

| | Weight Loss | | Occult Blood or |
|---|---|---|---|
| Score | (%) | Stool Consistency* | Gross Bleeding |
| 0 | None | Normal | Negative |
| 1 | 1-5 | Loose Stool | Negative |
| 2 | 5-10 | Loose Stool | Hemoccult Positive |
| 3 | 10-15 | Diarrhea | Hemoccult Positive |
| 4 | >15 | Diarrhea | Gross Bleeding |

^{#}DAI - (combined score of weight loss, stool consistency, and bleeding)/3.
*Normal stool - well formed pellets; loose stools - pasty stool that does not stick to the anus; and diarrhea - liquid stools that sticks to the anus.

On the last day of the study, animals were euthanized. Abdomen was open and the colon was sectioned at the level of the caecum and the rectum. The colon was weighted and its length was measured. The whole column was excised, slited longitudinally, and examined under a magnifying lens. Any visible damage was recorded and scored for gross pathology according to Wong [Wong et al., J. Pharm. Exp. Ther. 274: 475-80, 1995]. Namely, a score of 0 indicates no damage, 1 indicates localized hyperemia and/or edema, 2 indicates at least two sites of hyperemia and/or edema, 3 indicates localized erosion, 4 indicates localized ulcer and 5 indicates either an erosion site or ulcer extending for more than 2 cm along the colon or at least two sites of erosion or ulcer. Finally, the entire colon was fixed in 4% formaldehyde for histopathological evaluation.

The clinical outcome and gross pathology findings were analyzed using analysis of variance (ANOVA) followed by Fisher's post-hoc test. A value of p<0.05 is considered statistically significant.

Each treatment group comprised at least seven animals. The following groups served as negative controls: naïve animals, sham animals that received 70 μl of 50% ethanol without TNBS, and TNBS challenged untreated and vehicle treated animals. A group of animals receiving 10 mg/kg sulfasalazine served as positive controls. Sulfasalazine is a standard anti-inflammatory drug used for the treatment of mild to moderate ulcerative colitis and Crohn's disease, and as adjunctive therapy in the treatment of severe ulcerative colitis. This drug is also used for the treatment of non-IBD related disorders, such as rheumatoid arthritis and ankylosing spondylitis. The recognized side effect of this medicament is its hepatotoxicity upon chronic treatment.

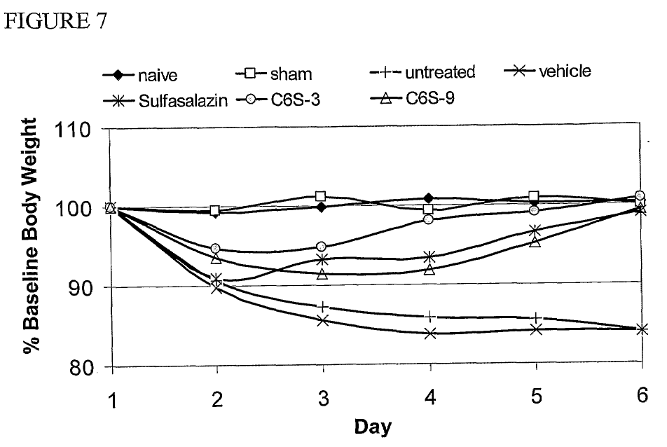

Results were expressed as percent of baseline body weight on Day 1 and are depicted in FIG. 7. Naïve and sham animals displayed a similar pattern and maintained during the period of the study their original body weight with minor fluctuations not exceeding 1%. Untreated and vehicle treated animals were similarly affected by rectal exposure to TNBS and displayed a regular loss in body weight of about 10% already one day after IBD induction and of 16% on Day 6. Animals treated with 10 mg/kg sulfasalazine displayed a transient loss of 10% in body weight one day after TNBS instillation. After 3 days of treatment the loss in body weight was halted and reversed, and animals regained normal weight on Day 6. Animals treated with 20 mg/kg of compounds C6S-3 and C6S-9 were significantly protected against weight loss and mortality. Animals treated with C6S-3 displayed a transient loss of only about 5% in body weight one day after TNBS instillation, whereas animals treated with compound C6S-9 behaved as the sulfasalazin treated group. After 4 days of treatment, the animals treated with compound C6S-3 already regained normal weight.

The inflammatory bowel disease developed in this model was rather severe, with mortality reaching 60% in the group of the thirty untreated animals during the period of the study. The compounds of the invention, C6S-3 and C6S-9, as well as the positive control sulfasalazine, dramatically reduced mortality to 7% (one of fourteen animals), 0% and 0%, respectively.

These results demonstrate that compounds of the invention have anti-inflammatory and gastro-protective activity in vivo in a model of inflammatory bowel disease. The compounds are at least as potent as the drug presently used, sulfasalazine. Side effects of sulfasalazine are well known and compounds of the invention may advantageously replace it. In addition, these results indicate that the compounds of the invention can be useful in the treatment of disorders having autoimmune etiology.

### Example 23

### Effect on Oxazolone Induced Delayed Type Hypersensitivity

The delayed type hypersensitivity (DTH) reaction is mediated by the cellular arm of the immune system. Dermal application of an inducer, the nature of which can be varied, elicits a response which generally includes induration, swelling and monocytic infiltration into the site of the lesion within 24 to 72 hours. The present study tests the immunoregulatory activity of compounds of the invention on oxazolone induced DTH.

Male ICR mice (average body weight 20-25 g, Harlan, Israel) were anesthetized using a mixture of 35 mg/kg ketamine and 8 mg/kg xylazine. The abdomen of the sedated animals was shaved, and the animals were sensitized by topical application of 2% oxazolone in acetone:sesame oil (4:1 volume per volume), 100 μl to the shaved abdomen and 5 μl to each paw. Five days later, the sensitized mice were anesthetized again and their right ear was challenged with 10 μl of 1% oxazolone. Immediately before this challenge, vehicle (5 ml/kg) or test compounds were administered i.v. Each treatment group comprised at least 5 animals. The ear thickness was measured 24 and 48 hours following challenge using a micrometer. The difference in ear thickness (ΔThickness) between the challenged and non-challenged ear was calculated. The average ΔThickness and SEM were calculated on each day following challenge (baseline) for all treatment groups. Results are reported in Table 7 below.

TABLE 7
ΔThickness in DTH.

| | Difference in Ear Thickness (×10⁻² mm) | | | | | |
|---|---|---|---|---|---|---|
| | Baseline | | Day 1 | | Day 2 | |
| Treatment | Mean | SEM | Mean | SEM | Mean | SEM |
| Untreated | 3.76 | 0.01 | 11.50 | 0.14 | 11.80 | 0.14 |
| Vehicle | 3.62 | 0.07 | 10.96 | 1.15 | 8.50 | 0.27 |
| C6S-3 (2 mg/kg IV) | 3.76 | 0.17 | 8.64 | 0.17 | 5.78 | 0.36 |
| C7S-2 (0.5 mg/kg IV) | 3.66 | 0.07 | 8.18 | 0.51 | 5.90 | 0.34 |

Untreated and vehicle treated animals displayed a similar pattern of increase in ΔThickness following oxazolone challenge of sensitized animals, implying a worsening of the immune status of the challenged ear. On day one after challenge, animals treated with either 2 mg/kg i.v. C6S-3 or 0.5 mg/kg i.v. C7S-2 already displayed a decrease in ΔThickness representing 25% and 29% inhibition as compared to untreated animals. On the second day following challenge, this trend was significantly strengthened and both compounds reduced the ΔThickness by more than 50%, implying an improvement of the challenged ear in these treated groups.

These results demonstrate that compounds of the invention have immunomodulatory activity in short term models of immune dysregulation.

### Example 24

### Safety

In the present study, impact of compounds of the invention on the central nervous system was measured by monitoring the body temperature, catalepsy and spontaneous locomotor activity of rodents. These activities are part of the Tetrad assays which are indicative, if all parameters are affected, of CB₁ mediated activity. In addition, lack of CNS psychomimetic effect was assessed in the elevated plus maze model. Finally, sensitivity to touch was determined in order to evaluate peripheral CB₁ related activity.

ICR male mice (average body weight 25 g, Harlan, Israel) were administered the compounds of the invention i.v. at a dose of 2 mg/kg and at a volume dose of 5 ml/kg. The psychoactive cannabinoid HU-210 was used as positive control at the 100-fold lower dose of 0.02 mg/kg i.v. The following measurements were made starting 15 minutes after compound administration. All tests were completed for each animal within approximately 10 minutes. Rectal temperature was monitored using a thermistor probe (YSI model 400, USA). Spontaneous locomotion was assessed using the open field methodology. The number of squares crossed by the animals were recorded and analyzed during a period of three minutes. At the end of the open field test, the animals were tested for catalepsy symptoms. This was carried out by gently forcing the animal to stand on its hind paws when its front paws are holding on an elevated beam. The time for the animal to step down of the beam was measured in seconds. A normal animal withdraws the beam immediately whereas cataleptic animal tend to stay on the beam. The longer the animal stays leaning on the beam, the more cataleptic the animal is. The behavior of the animals was then assessed in the elevated plus maze. The elevated plus maze consists of two open arms and two arms that are enclosed by high walls (arms 30×10 cm, wall height 20 cm). The elevated plus maze is usually elevated 80 cm above the floor. The mouse is placed on the maze head facing an open arm, and the time spent in the different compartments of the maze (open arms, closed arms and central area) are measured for the next 5 minutes. Results are expressed as percent of the time spent in the open arms, normal animals preferring to stay in the closed arms of the maze. Finally, animals were gently touched by an observer blinded for the treatment group and their sensitivity scored according to the following scale: 0 not sensitive, 1 sensitive and 2 highly sensitive. Results are expressed as average±SEM. At the end of the study, the animals were euthanized.

None of C5S-1, C5S-2, C6S-2, C6S-3, C6S-5, C6S-7, C6S-8, C7S-1, C6M-7, and C6M-9, displayed adverse cannabimimetic activities in any of the parameters monitored at the dose of 2 mg/kg i.v. HU-210, which served as control at a 100-fold lower dose, confirmed the validity of these models for the assessment of CB₁ related psychomimetic activity.

For instance, over a period of three minutes naïve animals crossed on average 73.38±13.69 squares and vehicle treated animals displayed a highly similar behavior with an average of 75.63±6.24 crossed squares. Compounds of the invention did not affect spontaneous locomotor activity and the most “active” compound, C6S-3, decreased non-significantly the number of squares crossed by only 13%, with an average of 65.80±7.04. For comparison, animals injected the psychoactive control HU-210 at a 100-fold lower dose displayed a significantly impaired locomotor activity and crossed more than 50% less squares with an average of only 32.33±10.21.

Similarly, naïve animals and vehicle treated animals had a rectal temperature of 38.68±0.25° C. and 38.88±0.11° C., respectively. Compounds of the invention did not affect the rectal temperatures of the animals and the most “hypothermic” compound, C7S-1, decreased non-significantly the temperature by about 1.2° C., with an average of 37.45±0.39° C. which constitutes a normal body temperature. For comparison, HU-210 at 0.02 mg/kg i.v. caused statistically significant hypothermia with a drop of about 2° C. to 36.73±0.29° C.

Compounds of the invention did not cause cataleptic behavior. The lack of central CB₁ related activity was confirmed by the fact that all compound treated animals displayed the normal preference toward the closed arms of the maze and spent no more than 25% of their time in the open arms. For comparison, vehicle treated animals spent about 15% of the time in the open arms, whereas animals injected with HU-210 spent significantly more time with an average of almost 41%. Finally, none of the compounds tested caused sensitivity to touch, a parameter which indicates lack of more peripheral CB₁ related activity, whereas HU-210 injected animals scored on average above 1 showing certain tactile sensitivity.

Compound C7S-17 was tested at increasing doses ranging from 10 to 40 mg/kg i.v. At the highest dose tested the sole parameter that was somehow affected by the administration of the compound was the rectal temperature, which was 0.5 hr after injection about 0.9° C. below controls. Still the average of 37.84±0.62° C. achieved by this group is considered within normal range and the drop is not statistically significant. Three hours after injection, animals administered 40 mg/kg of C7S-17 were back to baseline.

Thus, compounds of the invention are devoid of deleterious cannabimimetic effects at a dose where therapeutic benefice was previously shown. Moreover, compounds of the invention are at least 100-fold safer than the psychoactive control HU-210. Finally, it is interesting to note that some of the compounds tested were previously found to bind the hCB₁ receptor with IC₅₀ and Kᵢ in the nanomolar range. Despite these findings regarding the affinity to hCB₁, these compounds seem devoid of cannabimimetic activity in the above-mentioned assays.

### Example 25

### Tolerance

Another concern often associated with cannabinoid compounds is the development of tolerance toward the positive effect of the compound, implying either a decrease in efficacy over time or conversely the need to increase the dose being administered to maintain a similar level of efficacy. In order to ascertain that compounds of the invention do not cause the development of tolerance, they were tested following repeated administration in the model of Visceral Pain previously described.

Briefly compounds of the invention were administered at 10 mg/kg i.p. daily for 10 days and their analgesic activity was tested on day 11 as described in Example 17 (namely at 2 mg/kg i.v.). Morphine, an analgesic compound known to induce tolerance was used as control. Each treatment group comprised 10 animals. On day 11, vehicle treated animals displayed on average 29.75±1.09 writhing responses. Animals repeatedly administered morphine for 10 days displayed on average 9.60±2.85 writhing responses when challenged with acetic acid on day 11, whereas animals repeatedly administered C7S-2 were still highly responsive to the analgesic activity of the compound on day 11 with 2.45±1.49 writhing responses. In other words, morphine that totally eliminated the pain response when administered once at 2 mg/kg, lost activity over repeated administration and reduced pain response by only 68% on the 1 μl day of administration. On the other hand, C7S-2 that also inhibited 100% of the writhing responses when administered once at 2 mg/kg, retained its potency following repeated administration with a lasting reduction of 92% in pain response.

These results demonstrate that compounds of the invention do not induce the development of tolerance, supporting their safety.

To the extent necessary to understand or complete the disclosure of the present invention, all publications, patents, and patent applications mentioned herein are expressly incorporated in their entirety by reference therein to the same extent as though each were individually so incorporated.

Although the present invention has been described with respect to various specific embodiments presented thereof for the sake of illustration only, such specifically disclosed embodiments should not be considered limiting. Many other such embodiments will occur to those skilled in the art based upon applicants' disclosure herein, and applicants propose to be bound only by the spirit and scope of their invention as defined in the appended claims.

1.-41. (canceled)

42. A compound of the general formula (I) or (II): 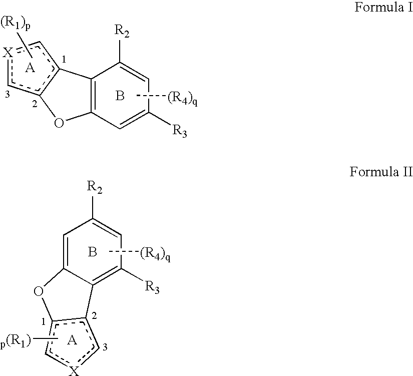
wherein [symbol]
represents a single or double bond;
X is (CHₘ)ₙ wherein m is an integer from 0 to 2 and n is an integer from 0 to 4;
R₁ is at each occurrence selected independently from the group consisting of:
a) a halogen;
b) a carbonyl;
c) an aryl;
d) Rₐ wherein Rₐ is selected from the group consisting of R_{b}, OR_{b}, C(O)OR_{b} and OC(O)R_{b} wherein R_{b} is a saturated or unsaturated, linear or branched C₁-C₈ alkyl substituted with one or more heteroatoms selected from the group consisting of N, O and S;
e) Rₑ wherein Rₑ is selected from R, OR, OC(O)OR, C(O)OR, OC(O)R and OC(O)N(R′)₂, wherein R is selected from the group consisting of a hydrogen, a saturated or unsaturated, linear, branched or cyclic C₁-C₆ alkyl, C₁-C₆ alkyl-OR, C₁-C₆ alkyl-(OR)₂, C₁-C₆ alkyl-C(O)OR, and C₁-C₆ alkyl-C(O)N(R′)₂, and wherein R′ is at each occurrence independently selected from the group consisting of a hydrogen and a saturated or unsaturated, linear, branched or cyclic C₁-C₆ alkyl;
f) an oxime; and
g) N(R′)₂, wherein R′ is at each occurrence as previously defined;
p is an integer from 0 to 14;
R₂ is selected from the group consisting of:
a) a hydrogen;
b) Rₐ or Rₑ, wherein Rₐ and R_{c} are as previously defined; and
c) OR″Z, wherein R″ is selected from the group consisting of a direct bond, C(O), Rₑ and C(O)Rₑ wherein Rₑ is a saturated or unsaturated, linear or branched C₁-C₈ alkyl, and Z is selected from the group consisting of ONO₂, a halogen, P(O)(OR)₂, SR, S(O)R′, S(O)(O)R′, N(R′)₂, wherein R′ is as previously defined, and a saturated or unsaturated heterocyclic ring of up to 6 atoms containing at least one heteroatom selected from the group consisting of N, O and S;
R₃ is selected from the group consisting of:
a) R_{d} wherein R_{d} is selected from the group consisting of hydrogen, C(O)OR′″, C(O)R′″, CN and NO₂, wherein R′″ is selected from the group consisting of a hydrogen and a saturated or unsaturated, linear, branched or cyclic C₁-C₁₂ alkyl;
b) a saturated or unsaturated, linear, branched or cyclic C₂-C₁₂ alkyl which is unsubstituted or substituted by a saturated or unsaturated heterocyclic ring as previously defined;
c) a saturated or unsaturated, linear or branched C₁-C₁₂ alkyl substituted by an aryl; and
d) a saturated or unsaturated heterocyclic ring as previously defined, said ring being unsubstituted or substituted by at least one saturated or unsaturated, linear branched or cyclic C₁-C₆ alkyl, wherein said alkyl can be unsubstituted or substituted by an aryl; and
R4 is selected independently at each occurrence from the group consisting of hydrogen, NO₂ and NH₂; and q is an integer from 0 to 2;
and stereoisomers, pharmaceutically acceptable salts, esters, polymorphs or solvates of said compounds;
with the provisos that A is not a phenyl ring and that in compounds of formula (I):
(a) when n is 1, R₁ is not a phenyl at position C2;
(b) when n is 2, and R₁ at C2 is isopropyl then R₁ at C5 is other than methyl; and
(c) when n is 2, R₁ is methyl and hydroxyl at C3 and isopropenyl at C6, then R₂ is other than OH, OCH₃ and OC(O)CH₃;
and that in compounds of formula (II) when n is 2, and R₁ at C2 is isopropyl then R₁ at C5 is other than methyl.

43. The compound of claim 42, represented by the structure of formula (I):

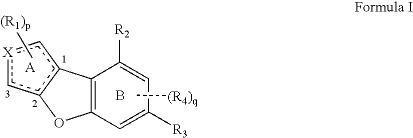

44. The compound of claim 43 wherein n is an integer from 1 to 3, p is an integer from 0 to 4, q is an integer from 0 to 2, ring A is saturated or unsaturated wherein the optional double bond on ring A is positioned between C1 and C2 or C3 and C4, R₁ is at each occurrence independently selected from the group consisting of hydrogen, halogen, carbonyl, oxime, NH₂, R, OR and C(O)OR; R₂ is selected from the group consisting of hydrogen, R_{c}, OR, OR″Z, OC(O)R_{b}, OR_{b} and OC(O)R; R₃ is selected from the group consisting of C(O)R′″, C(O)OR′″, and a saturated or unsaturated, linear, branched or cyclic C₁-C₁₂ alkyl which is unsubstituted or substituted by a heterocyclic ring or by an aryl; and R₄ is selected from the group consisting of hydrogen and NO₂, wherein R, R″, R′″, R_{b}, heterocyclic ring and Z are as previously defined.

45. The compound of claim 44, selected from the group consisting of:
a) a compound of formula (I) wherein n is 1, ring A is saturated, R₁ is at each occurrence independently selected from the group consisting of hydrogen and CH₃; R₂ is selected from the group consisting of OH and OC(O)CH═CHC(O)OH; and R₃ is selected from the group consisting of 1,1-dimethylpentyl and 1,1-dimethylheptyl;
b) a compound of formula (I) wherein n is 2, ring A is saturated or unsaturated wherein the optional double bond is positioned between C1 and C2 or C3 and C4, R₁ is at each occurrence independently selected from the group consisting of hydrogen, carbonyl, OH, isopropylidene, oxime, iodine and CH₃; R₂ is selected from the group consisting of OH, OCH₃, OCH₂C(O)OH, OCH₂SCH₃, OP(O)(OH)₂, OC(O)CH₃, OP(O)(OC₂H₅)₂, OCH₂-tetrazole, OCH₂CH₂-morpholine, OC(O)-piperidine, OC(O)(CH₂)₂NHCH₃, OCH₂CH(OH)CH₂OH, OC(O)CH═CHC(O)OH, OC(O)(CH₂)₃Br and OC(O)(CH₂)₃ONO₂; R₃ is selected from the group consisting of 2-phenethyl-[1,3]-dithiolane, 2-methyl-[1,3]dithiolan-2-yl, C(O)CH₃, C(O)OCH₃, 1,1-dimethylpentyl and 1,1-dimethylheptyl; and R₄ is selected from the group consisting of hydrogen and NO₂; and
c) a compound of formula (I) wherein n is 3, ring A is saturated, R₁ is selected from the group consisting of hydrogen, OH, iodine, oxime, C(O)OCH₃, NH₂, OC(O)CH═CHC(O)OH, C(O)OCH₃, CH₂C(O)OCH₃, C(O)OH, CH₂OH, CH₃ and carbonyl; R₂ is selected from the group consisting of hydrogen, OH, OCH₂CH₂-morpholine, OCH₂C(O)OH, OC(O)CH═CHC(O)OH, OCH₂-tetrazole, OP(O)(OH)₂, OCH₂C(O)N(C₂H₅)₂, OC(O)CH₂OCH₂CH₂OCH₂CH₂OCH₃ and O(CH₂)₃C(O)OH; and R₃ is selected from the group consisting of pentyl, 1,1-dimethylpentyl and 1,1-dimethylheptyl; and R₄ is selected from the group consisting of hydrogen and NO₂.

46. The compound of claim 45, selected from the group consisting of:
a) a compound of formula (I) wherein n is 1, ring A is saturated, and
i) R₁ is selected from the group consisting of hydrogen, CH₃ at position C2, and CH₃ at positions C2 and C3; R₂ is OH and R₃ is 1,1-dimethylheptyl;
ii) R₁ is CH₃ at position C2, R₂ is OH and R₃ is 1,1-dimethylpentyl; or
iii) R₁ is CH₃ at positions C2 and C3, R₂ is OC(O)CH═CHC(O)OH and R₃ is 1,1-dimethylheptyl;
b) a compound of formula (I) wherein n is 2, ring A is saturated, and
i) R₁ is selected from the group consisting of hydrogen, OH, carbonyl, iodine or oxime at position C3, gem-dimethyl at position C4, both CH₃ at position C2 and isopropylidene at position C5, both carbonyl at position C3 and gem-dimethyl at position C4, and both OH at position C3 and gem-dimethyl at position C4; R₂ is OH, and R₃ is 1,1-dimethylheptyl;
ii) R₁ is selected from the group consisting of hydrogen, OH, carbonyl or oxime at position C3, with or without a further gem-dimethyl at position C4, iodine at position C3 and gem-dimethyl at position C4; R₂ is OH, and R₃ is 1,1-dimethylpentyl;
iii) R₁ is hydrogen or gem-dimethyl at position C4, R₂ is OCH₂C(O)OH, and R₃ is 1,1-dimethylheptylor 1,1-dimethylpentyl;
iv) R₁ is hydrogen, R₂ is selected from the group consisting of OCH₂CH(OH)CH₂OH, OC(O)CH═CHC(O)OH, OC(O)CH₃, OP(O)(OH)₂, OP(O)(OC₂H₅)₂,OCH₂-tetrazole, OC(O)-piperidine, OC(O)(CH₂)₃Br and OC(O)(CH₂)₃ONO₂, and R₃ is 1,1-dimethylpentyl;
v) R₁ is hydrogen, R₂ is OH, and R₃ is selected from the group consisting of 2-methyl-[1,3]dithiolan-2-yl, C(O)CH₃, and C(O)OCH₃;
vi) R₁ is selected from the group consisting of carbonyl or oxime at position C3 with or without a further gem-dimethyl at position C4; R₂ is OCH₂SCH₃, and R₃ is 1,1-dimethylpentyl;
vii) R₁ is gem-dimethyl at position C4, R₂ is OC(O)CH═CHC(O)OH or OC(O)(CH₂)₂NHCH₃, and R₃ is 1,1-dimethylpentyl;
viii) R₁ is gem-dimethyl at position C4, R₂ is OH or OC(O)CH═CHC(O)OH, and R₃ is 2-phenethyl-[1,3]dithiolan-2-yl; or
ix) R₁ is OH at position C3, R₂ is OC(O)CH₂—O—(CH₂)₂—O—(CH₂)₂OCH₃, and R₃ is 1,1-dimethylheptyl;
c) a compound of formula (I) wherein n is 2, ring A is unsaturated with a double bond positioned between C3 and C4, R₁ is hydrogen, R₂ is OH, and R₃ is 1,1-dimethylpentyl or 1,1-dimethylheptyl;
d) a compound of formula (I) wherein n is 2, ring A is saturated, R₁ is hydrogen, R₂ is OH, and R₃ is 1,1-dimethylpentyl and R₄ is NO₂ either at ortho, para, or both ortho and para position to R₂;
e) a compound of formula (I) wherein n is 2, ring A is unsaturated with a double bond positioned between C1 and C2, and
i) R₁ is hydrogen, R₂ is OH or OC(O)CH═CHC(O)OH, and R₃ is 1,1-dimethylpentyl or 1,1-dimethylheptyl;
ii) R₁ is a carbonyl at position C3 and gem-dimethyl at position C6, R₂ is OH or OCH₃, and R₃ is 1,1-dimethylheptyl;
iii) R₁ is a carbonyl at position C3 and gem-dimethyl at position C5, R₂ is OCH₃, and R₃ is 1,1-dimethylheptyl; or
iv) R₁ is gem-dimethyl at position C4, R₂ is OH, and R₃ is 1,1-dimethylheptyl;
f) a compound of formula (I) wherein n is 3, ring A is saturated, and
i) R₁ is selected from the group consisting of hydrogen, carbonyl and OH at position C3, R₂ is OH, and R₃ is 1,1-dimethylheptyl or dimethylpentyl;
ii) R₁ is carbonyl at position C3 or OH at both positions C3 and C4, R₂ is OH, and R₃ is 1,1-dimethylpentyl;
iii) R₁ is hydrogen, R₂ is OCH₂CH₂-morpholine, and R₃ is 1,1-dimethylpentyl or 1,1-dimethylheptyl;
iv) R₁ is hydrogen, R₂ is OCH₂C(O)OH or OC(O)CH═CHC(O)OH, and R₃ is 1,1-dimethylheptyl;
v) R₁ is OH at position C3, R₂ is selected from the group consisting of OCH₂C(O)OH, OP(O)(OH)₂, O(CH₂)₃C(O)OH, OCH₂C(O)N(C₂H₅)₂, O(CH₂)-2-morpholine and OCH₂-tetrazole, and R₃ is 1,1-dimethylheptyl;
vi) R₁ is iodine or OC(O)CH═CHC(O)OH at position C3, R₂ is OC(O)CH═CHC(O)OH, and R₃ is 1,1-dimethylheptyl;
vii) R₁ is hydrogen or OH at position C3, R₂ is OH, and R₃ is pentyl;
viii) R₁ is selected from the group consisting of oxime, iodine, or NH₂ at position C3; C(O)OCH₃, CH₂OH, CH₂C(O)OCH₃ or C(O)OH at position C7; and both OH at position C3 and C(O)OH at position C7, R₂ is OH, and R₃ is 1,1-dimethylheptyl;
ix) R₁ is NH₂ at position C3, R₂ is hydrogen, and R₃ is 1,1-dimethylheptyl; or
x) R₁ is OH at position C3, R₂ is OH, R₃ is 1,1-dimethylheptyl and R₄ is NO₂ either at ortho or para position to R₂.

47. The compound of claim 46, wherein said compound is selected from the group consisting of: 6-(1,1-dimethylpentyl)-8a-methyl-2,3,3 a,8a-tetrahydro-1H-8-oxa-cyclo-penta[α]inden-4-ol; 6-(1,1-dimethylheptyl)-8a-methyl-2,3,3a,8a-tetrahydro-1H-8-oxa-cyclo-penta[α]inden-4-ol; 6-(1,1-dimethylheptyl)-2,3,3a,8a-tetrahydro-1H-8-oxa-cyclopenta[α]inden-4-ol; 6-(1,1-dimethylheptyl)-1,8a-dimethyl-2,3,3a,8a-tetrahydro-1H-8-oxa-cyclo-penta[α]inden-4-ol; but-2-enedioic acid mono-[6-(1,1-dimethylheptyl)-1,8a-dimethyl-2,3,3a,8a-tetrahydro-1H-8-oxa-cyclopenta[α]inden-4-yl]ester; 3-(1,1-dimethyl-heptyl)-5a,6,7,8,9,9a-hexahydro-dibenzofuran-1-ol; 3-(1,1-dimethylheptyl)-6-iodo-5a,6,7,8,9,9a-hexahydro-dibenzofuran-1-ol; 3-(1,1-dimethylheptyl)-5a,8,9,9a-tetrahydro-dibenzofuran-1-ol; 3-(1,1-dimethylpentyl)-5a,6,7,8,9,9a-hexahydro-dibenzofuran-1-ol; 3-(1,1-dimethylpentyl)-6-iodo-5a,6,7,8,9,9a-hexahydro-dibenzofuran-1-ol; 3-(1,1-dimethyl-pentyl)-5a,8,9,9a-tetrahydro-dibenzofuran-1-ol; 3-(1,1-dimethylheptyl)-5a,6,7,8,9,9a-hexahydro-dibenzofuran-1,6-diol; 3-(1,1-dimethylpentyl)-7,7-dimethyl-5a,6,7,8,9,9a-hexa-hydro-dibenzofuran-1-ol; 3-(1,1-dimethylpentyl)-7,7-dimethyl-5a,6,7,8,9,9a-hexahydro-dibenzofuran-1,6-diol; 3-(1,1-dimethylheptyl)-7,7-dimethyl-5a,6,7,8,9,9a-hexahydro-dibenzofuran-1,6-diol; [3-(1,1-dimethylpentyl)-5a,6,7,8,9,9a-hexahydro-dibenzofuran-1-yloxy]-acetic acid; 3-[3-(1,1-dimethylpentyl)-5a,6,7,8,9,9a-hexahydro-dibenzofuran-1-yloxy]-propane-1,2-diol; 3-(1,1-dimethylpentyl)-5a,6,7,8,9,9a-hexahydro-dibenzofuran-1,6-diol; 3-(2-methyl-[1,3]dithiolan-2-yl)-5a,6,7,8,9,9a-hexahydro-dibenzofuran-1-ol; 4-{2-[3-(1,1-dimethylpentyl)-5a,6,7,8,9,9a-hexahydro-dibenzofuran-1-yloxy]-ethyl}-morpholine; but-2-enedioic acid mono-[3-(1,1-dimethylpentyl)-5a,6,7,8,9,9a-hexahydro-dibenzofuran-1-yl]ester; acetic acid 3-(1,1-dimethylpentyl)-5a,6,7,8,9,9a-hexahydro-dibenzofuran-1-yl ester; diethyl phosphoric acid mono-[3-(1,1-dimethylpentyl)-5a,6,7,8,9,9a-hexahydro-dibenzo-furan-1-yl]ester; phosphoric acid mono-[3-(1,1-dimethylpentyl)-5a,6,7,8,9,9a-hexahydro-dibenzofuran-1-yl]ester; 3-(1,1-dimethylheptyl)-7,7-dimethyl-5a,6,7,8,9,9a-hexahydro-dibenzofuran-1-ol; [3-(1,1-dimethylheptyl)-5a,6,7,8,9,9a-hexahydro-dibenzofuran-1-yloxy]-acetic acid; 3-(1,1-dimethylheptyl)-8-isopropylidene-5a-methyl-5a,6,7,8,9,9a-hexahydro-dibenzofuran-1-ol; 1-(1-hydroxy-5a,6,7,8,9,9a-hexahydro-dibenzofuran-3-yl)-ethanone; 1-hydroxy-5a,6,7,8,9,9a-hexahydro-dibenzofuran-3-carboxylic acid methyl ester; 5-[3-(1,1-dimethylpentyl)-5a,6,7,8,9,9a-hexahydro-dibenzofuran-1-yloxymethyl]-1H-tetrazole; piperidine-3-carboxylic acid 3-(1,1-dimethylpentyl)-5a,6,7,8,9,9a-hexahydro-dibenzofuran-1-yl ester; 4-bromobutyric acid 3-(1,1-dimethylpentyl)-5a,6,7,8,9,9a-hexahydro-dibenzofuran-1-yl ester; 4-nitrooxy-butyric acid 3-(1,1-dimethylpentyl)-5a,6,7,8,9,9a-hexahydro-dibenzofuran-1-yl ester; 7-(1,1-dimethylheptyl)-9-hydroxy-3,3-dimethyl-2,3,4a,9b-tetrahydro-1H-dibenzofuran-4-one; 7-(1,1-dimethylheptyl)-9-hydroxy-2,3,4a,9b-tetrahydro-1H-dibenzofuran-4-one; 7-(1,1-dimethylheptyl)-9-hydroxy-2,3,4a,9b-tetrahydro-1H-dibenzofuran-4-one oxime; 7-(1,1-dimethylpentyl)-9-methylsulfanylmethoxy-2,3,4a,9b-tetrahydro-1H-dibenzofuran-4-one; 7-(1,1-dimethylpentyl)-9-hydroxy-2,3,4a,9b-tetrahydro-1H-dibenzofuran-4-one; 7-(1,1-dimethylpentyl)-9-methylsulfanylmethoxy-2,3,4a,9b-tetrahydro-1H-dibenzofuran-4-one oxime; 7-(1,1-dimethylpentyl)-9-hydroxy-2,3,4a,9b-tetrahydro-1H-dibenzofuran-4-one oxime; 7-(1,1-dimethylpentyl)-9-hydroxy-3,3-dimethyl-2,3,4a,9b-tetrahydro-1H-dibenzofuran-4-one; 7-(1,1-dimethylpentyl)-3,3-dimethyl-9-methylsulfanylmethoxy-2,3,4a,9b-tetrahydro-1H-dibenzofuran-4-one; 7-(1,1-dimethylpentyl)-9-hydroxy-3,3-dimethyl-2,3,4a,9b-tetrahydro-1H-dibenzofuran-4-one oxime; 7-(1,1-dimethylpentyl)-3,3-dimethyl-9-methylsulfanylmethoxy-2,3,4a,9b-tetrahydro-1H-dibenzofuran-4-one oxime; [3-(1,1-dimethylheptyl)-7,7-dimethyl-5a,6,7,8,9,9a-hexahydro-dibenzofuran-1-yloxy]-acetic acid; but-2-enedioic acid mono-[3-(1,1-dimethylheptyl)-7,7-dimethyl-5a,6,7,8,9,9a-hexahydro-dibenzofuran-1-yl]ester; 7,7-dimethyl-3-(2-phenethyl-[1,3]dithiolan-2-yl)-5a,6,7,8,9,9a-hexahydro-dibenzofuran-1-ol; 3-methylamino-propionic acid 3-(1,1-dimethyl-heptyl)-7,7-dimethyl-5a,6,7,8,9,9a-hexahydro-dibenzofuran-1-yl ester; but-2-enedioic acid mono-[7,7-dimethyl-3-(2-phenethyl-[1,3]dithiolan-2-yl)-5a,6,7,8,9,9a-hexahydro-dibenzofuran-1-yl]ester; 3-(1,1-dimethylpentyl)-2,4-dinitro-5a,6,7,8,9,9a-hexahydro-dibenzofuran-1-ol; 3-(1,1-dimethylpentyl)-2-nitro-5a,6,7,8,9,9a-hexahydro-dibenzofuran-1-ol; 3-(1,1-dimethylpentyl)-4-nitro-5a,6,7,8,9,9a-hexahydro-dibenzofuran-1-ol; 2-(1,1-dimethylheptyl)-5,6,7,8,9,9a-hexahydro-4bH-10-oxa-benzo[α]azulen-4-ol; 2-(1,1-dimethyl-heptyl)-5,6,7,8,9,9a-hexahydro-4bH-10-oxa-benzo[α]azulen-4,9-diol; 2-(1,1-dimethyl-pentyl)-5,6,7,8,9,9a-hexahydro-4bH-10-oxa-benzo[α]azulen-4-ol; 2-(1,1-dimethylpentyl)-5,6,7,8,9,9a-hexahydro-4bH-10-oxa-benzo[α]azulen-4,9-diol; 2-(1,1-dimethylpentyl)-4-hydroxy-4-b,5,6,7,8,9a-hexahydro-10-oxa-benzo[α]azulen-9-one; 2-(1,1-dimethylheptyl)-4-hydroxy-4-b,5,6,7,8,9a-hexahydro-10-oxa-benzo[α]azulen-9-one; 4-{2-[2-(1,1-dimethyl-heptyl)-5,6,7,8,9,9a-hexahydro-4bH-10-oxa-benzo[α]azulen-4-yloxy]-ethyl}-morpholine; [2-(1,1-dimethylheptyl)-5,6,7,8,9,9a-hexahydro-4bH-10-oxa-benzo[α]azulen-4-yloxy]-acetic acid; but-2-enedioic acid mono-[2-(1,1-dimethylheptyl)-5,6,7,8,9,9a-hexahydro-4bH-10-oxa-benzo[α]azulen-4-yl]ester; [2-(1,1-dimethylheptyl)-9-hydroxy-5,6,7,8,9,9a-hexahydro-4bH-10-oxa-benzo[α]azulen-4-yloxy]-acetic acid; 2-(1,1-dimethylheptyl)-5,6,7,9a-tetrahydro-4bH-10-oxa-benzo[α]azulen-4-ol; 2-(1,1-dimethylheptyl)-5,6,7,8,9,9a-hexahydro-4bH-10-oxa-benzo[α]azulen-4,8,9-triol; but-2-enedioic acid mono-[9-(3-carboxy-acryloyloxy)-2-(1,1-dimethylheptyl)-5,6,7,8,9,9a-hexahydro-4bH-10-oxa-benzo[α]azulen-4-yl]ester; phosphoric acid mono-[2-(1,1-dimethylheptyl)-9-hydroxy-5,6,7,8,9,9a-hexahydro-4bH-10-oxa-benzo[α]azulen-4-yl]ester; 2-pentyl-5,6,7,8,9,9a-hexahydro-4bH-10-oxa-benzo[α]azulen-4-ol; 2-pentyl-5,6,7,8,9,9a-hexahydro-4bH-10-oxa-benzo[α]azulen-4,9-diol; 4-[2-(1,1-dimethylheptyl)-9-hydroxy-5,6,7,8,9,9a-hexa-hydro-4bH-10-oxa-benzo[α]azulen-4-yloxy]-butyric acid; 2-[2-(1,1-dimethylheptyl)-9-hydroxy-5,6,7,8,9,9a-hexahydro-4bH-10-oxa-benzo[α]azulen-4-yloxy]-N,N-diethyl-acetamide; 2-(1,1-dimethylheptyl)-4-(2-morpholin-4-yl-ethoxy)-5,6,7,8,9,9a-hexahydro-4bH-10-oxa-benzo[α]azulen-9-ol; 2-(1,1-dimethylheptyl)-4-(2H-tetrazol-5-ylmethoxy)-5,6,7,8,9,9a-hexahydro-4bH-10-oxa-benzo[α]azulen-9-ol; 2-(1,1-dimethylheptyl)-4-hydroxy-5,6,7,8,9,9a-hexahydro-4bH-10-oxa-benzo[α]azulen-5-carboxylic acid methyl ester; 2-(1,1-dimethylheptyl)-4,9-dihydroxy-5,6,7,8,9,9a-hexahydro-4bH-10-oxa-benzo[α]azulen-5-carboxylic acid; 2-(1,1-dimethylheptyl)-5-hydroxymethyl-5,6,7,8,9,9a-hexahydro-4bH-10-oxa-benzo[α]azulen-4-ol; [2-(1,1-dimethylheptyl)-4-hydroxy-5,6,7,8,9,9a-hexahydro-4bH-10-oxa-benzo[α]azulen-5-yl]-acetic acid methyl ester; 2-(1,1-dimethylheptyl)-4-hydroxy-4b,5,6,7,8,9a-hexahydro-10-oxa-benzo[α]azulen-9-one oxime; 2-(1,1-dimethylheptyl)-9-iodo-5,6,7,8,9,9a-hexahydro-4bH-10-oxa-benzo[α]azulen-4-ol; [2-(2-methoxy-ethoxy)-ethoxy]-acetic acid 2-(1,1-dimethylheptyl)-9-hydroxy-5,6,7,8,9,9a-hexahydro-4bH-10-oxa-benzo[α]azulen-4-yl ester; but-2-enedioic acid mono-[2-(1,1-dimethylheptyl)-9-iodo-5,6,7,8,9,9a-hexahydro-4bH-10-oxa-benzo[α]azulen-4-yl]ester; 2-(1,1-dimethylheptyl)-4-hydroxy-5,6,7,8,9,9a-hexahydro-4bH-10-oxa-benzo[α]azulen-5-carboxylic acid; 9-amino-2-(1,1-dimethylheptyl)-5,6,7,8,9,9a-hexahydro-4bH-10-oxa-benzo[α]azulen-4-ol; 9-amino-2-(1,1-dimethylheptyl)-5,6,7,8,9,9a-hexahydro-4bH-10-desoxy-benzo[α]azulen-4-ol; 2-(1,1-dimethylheptyl)-3-nitro-5,6,7,8,9,9a-hexahydro-4bH-10-oxa-benzo[α]azulen-4,9-diol; 2-(1,1-dimethylheptyl)-1-nitro-5,6,7,8,9,9a-hexahydro-4bH-10-oxa-benzo[α]azulen-4,9-diol; 3-(1,1-dimethylheptyl)-6,7,8,9-tetrahydro-dibenzofuran-1-ol; but-2-enedioic acid mono-[3-(1,1-dimethylheptyl)-6,7,8,9-tetrahydro-dibenzofuran-1-yl]ester; 3-(1,1-dimethylpentyl)-6,7,8,9-tetrahydro-dibenzofuran-1-ol; 7-(1,1-dimethylheptyl)-9-methoxy-1,1-dimethyl-2,3-dihydro-1H-dibenzofuran-4-one; 7-(1,1-dimethylheptyl)-9-methoxy-2,2-dimethyl-2,3-dihydro-1H-dibenzofuran-4-one; but-2-enedioic acid mono-[3-(1,1-dimethylpentyl)-6,7,8,9-tetrahydro-dibenzofuran-1-yl]ester; 7-(1,1-dimethylheptyl)-9-hydroxy-1,1-dimethyl-2,3-dihydro-1H-dibenzofuran-4-one; and 3-(1,1-dimethylheptyl)-7,7-dimethyl-6,7,8,9-tetrahydro-dibenzofuran-1-ol.

48. The compound of claim 42, represented by the structure of formula (II):

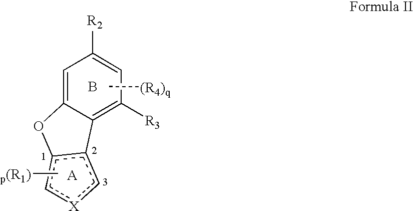

49. The compound of claim 48 wherein n is an integer from 1 to 3, ring A is unsaturated, R₁ is selected from the group consisting of hydrogen, carbonyl, and R; R₂ is OR, and R₃ is a saturated or unsaturated, linear, branched or cyclic C₁-C₁₂ alkyl, wherein R is as previously defined.

50. The compound of claim 49 wherein n is 2, ring A is unsaturated and the double bond is positioned between C1 and C2, R₂ is OCH₃ and R₃ is 1,1-dimethylheptyl, and further wherein
(a) R₁ is selected from the group consisting of hydrogen, carbonyl and CH₃; or
(b) R₁ is a carbonyl at position C6 and gem-dimethyl at position C3 or C4.

51. The compound of claim 50, selected from the group consisting of: 9-(1,1-dimethylheptyl)-7-methoxy-1,1-dimethyl-2,3-dihydro-1H-dibenzo-furan-4-one; and 9-(1,1-dimethylheptyl)-7-methoxy-2,2-dimethyl-2,3-dihydro-1H-dibenzofuran-4-one.

52. A pharmaceutical composition comprising as an active ingredient an effective amount of a compound of claim 1 and a pharmaceutically acceptable diluent or carrier.

53. The pharmaceutical composition of claim 52 wherein the diluent and comprises an aqueous solution of a pharmaceutically acceptable cosolvent, a micellar solution or emulsion prepared with natural or synthetic ionic or non-ionic surfactants, or a combination of such cosolvent and micellar or emulsion solutions.

54. The pharmaceutical composition of claim 52 wherein the carrier comprises a solution of ethanol, a surfactant and water or an emulsion comprising triglycerides, lecithin, glycerol, an emulsifier, and water.

55. The pharmaceutical composition of claim 52 in a unit dosage form suitable for oral or parenteral administration.

56. A method for preventing, alleviating or treating inflammation, autoimmune diseases, pain, neurological disorders, neurodegenerative diseases, neuroinflammatory conditions, ocular disorders, bone disorders, cardiovascular and cardio-inflammatory disorders, appetite disorders, emetic conditions and certain types of cancer, which comprises administering to a subject in need thereof a prophylactically and/or a therapeutically effective amount of a compound according to claim 42 or a pharmaceutical composition comprising the compound and a pharmaceutically acceptable diluent or carrier.

57. The method of claim 56 wherein the inflammation and autoimmune diseases are selected from the group comprising rheumatoid arthritis, juvenile arthritis, osteoarthritis, allergies and allergic reactions, multiple sclerosis, systemic lupus erythematosus, myasthenia gravis, diabetes mellitus type I, hepatitis, psoriasis, inflammatory bowel disease, Crohn's disease, ulcerative colitis, tissue rejection in organ transplants, malabsorption syndromes, celiac disease, pulmonary disease, asthma, chronic bronchitis, chronic obstructive pulmonary disease and Sjögren's syndrome.

58. The method of claim 56 wherein pain is selected from the group comprising acute, chronic, peripheral, visceral, neuropathic, inflammatory and referred pain.

59. The method of claim 56 wherein the neurological disorders, the neurodegenerative diseases and the neuroinflammatory conditions are selected from the group comprising stroke, migraine, cluster headache, epilepsy, Parkinson's disease, Alzheimer's disease, amyotrophic lateral sclerosis, Huntington's chorea, prion-associated diseases, poisoning of the central nervous system, muscle spasm and tremor, meningitis, encephalitis, cerebral ischemia, and Guillain-Barré syndrome.

60. The method of claim 56 wherein the cardiovascular and cardio-inflammatory disorders are selected from the group comprising atherosclerosis, pericarditis, myocarditis, endocarditis, arrhythmia, hypertension and myocardial ischemic damage.

61. The method of claim 56 wherein the bone, ocular, appetite disorders and emetic conditions are selected from the group consisting of abnormal bone metabolism, Paget's disease, osteoporosis, glaucoma, anorexia, cachexia, vomiting and nausea.

62. The method of claim 56 wherein the cancer is selected from the group consisting of malignant brain tumor, skin tumor, lung adenocarcinoma, uterus, breast and prostate carcinoma, lymphoma, glioma, thyroid epithelioma, and neuroblastoma.


SEQUENCE LISTING (16 sequences)

SEQ ID NO: 1 - DNA, 21 bp, Artificial sequence
  Single strand DNA oligonucleotide
ttccgtttctcgtggtcactt

SEQ ID NO: 2 - DNA, 20 bp, Artificial sequence
  Single strand DNA oligonucleotide
agcgctgaggttttcctgaa

SEQ ID NO: 3 - DNA, 21 bp, Artificial sequence
  Single strand DNA oligonucleotide
acactccttagtcctcggcca

SEQ ID NO: 4 - DNA, 20 bp, Artificial sequence
  Single strand DNA oligonucleotide
ccatcagaggcaaggaggaa

SEQ ID NO: 5 - DNA, 21 bp, Artificial sequence
  Single strand DNA oligonucleotide
gccctttgctatggtgtcctt

SEQ ID NO: 6 - DNA, 20 bp, Artificial sequence
  Single strand DNA oligonucleotide
tccctggtttctcttcccaa

SEQ ID NO: 7 - DNA, 20 bp, Artificial sequence
  Single strand DNA oligonucleotide
ttccaggtgcacacaggcta

SEQ ID NO: 8 - DNA, 21 bp, Artificial sequence
  Single strand DNA oligonucleotide
gcacgctgagtacctcattgg

SEQ ID NO: 9 - DNA, 17 bp, Artificial sequence
  Single strand DNA oligonucleotide
tcacagttgccggctgg

SEQ ID NO: 10 - DNA, 20 bp, Artificial sequence
  Single strand DNA oligonucleotide
tctttgggacacctgctgct

SEQ ID NO: 11 - DNA, 21 bp, Artificial sequence
  Single strand DNA oligonucleotide
aaggactcaaatgggctttcc

SEQ ID NO: 12 - DNA, 23 bp, Artificial sequence
  Single strand DNA oligonucleotide
cctcattctgagacagaggcaac

SEQ ID NO: 13 - DNA, 20 bp, Artificial sequence
  Single strand DNA oligonucleotide
tcgccattgccaaggagtag

SEQ ID NO: 14 - DNA, 20 bp, Artificial sequence
  Single strand DNA oligonucleotide
ggtcaccccatcagatggaa

SEQ ID NO: 15 - DNA, 21 bp, Artificial sequence
  Single strand DNA oligonucleotide
ggttgtctcctgcgacttcaa

SEQ ID NO: 16 - DNA, 20 bp, Artificial sequence
  Single strand DNA oligonucleotide
gtaggccatgaggtccacca